SEQUENCE LISTING (63 sequences)

SEQ ID NO: 1 - DNA, 1857 bp, Homo sapiens
gatttggggagttatgcgccagtgccccagtgaccgcgggacacggagaggggaagtctgcgttgtacataaggacctagggactccgagcttggcctgagaacccttggacgccgagtgcttgccttacgggctgcactcctcaactctgctccaaagcagccgctgagctcaactcctgcgtccagggcgttcgctgcgcgccaggacgcgcttagtacccagttcctgggctctctcttcagtagctgctttgaaagctcccacgcacgtcccgcaggctagcctggcaacaaaactggggtaaaccgtgttatcttaggtcttgtcccccagaacatgacctagaggtacctgcgcatgcagatggccgatgcagccacgatagccaccatgaataaggcagcaggcggggacaagctagcagaactcttcagtctggtcccggaccttctggaggcggccaacacgagtggtaacgcgtcgctgcagcttccggacttgtggtgggagctggggctggagttgccggacggcgcgccgccaggacatcccccgggcagcggcggggcagagagcgcggacacagaggcccgggtgcggattctcatcagcgtggtgtactgggtggtgtgcgccctggggttggcgggcaacctgctggttctctacctgatgaagagcatgcagggctggcgcaagtcctctatcaacctcttcgtcaccaacctggcgctgacggactttcagtttgtgctcaccctgcccttctgggcggtggagaacgctcttgacttcaaatggcccttcggcaaggccatgtgtaagatcgtgtccatggtgacgtccatgaacatgtacgccagcgtgttcttcctcactgccatgagtgtgacgcgctaccattcggtggcctcggctctgaagagccaccggacccgaggacacggccggggcgactgctgcggccggagcctgggggacagctgctgcttctcggccaaggcgctgtgtgtgtggatctgggctttggccgcgctggcctcgctgcccagtgccattttctccaccacggtcaaggtgatgggcgaggagctgtgcctggtgcgtttcccggacaagttgctgggccgcgacaggcagttctggctgggcctctaccactcgcagaaggtgctgttgggcttcgtgctgccgctgggcatcattatcttgtgctacctgctgctggtgcgcttcatcgccgaccgccgcgcggcggggaccaaaggaggggccgcggtagccggaggacgcccgaccggagccagcgcccggagactgtcgaaggtcaccaaatcagtgaccatcgttgtcctgtccttcttcctgtgttggctgcccaaccaggcgctcaccacctggagcatcctcatcaagttcaacgcggtgcccttcagccaggagtatttcctgtgccaggtatacgcgttccctgtgagcgtgtgcctagcgcactccaacagctgcctcaaccccgtcctctactgcctcgtgcgccgcgagttccgcaaggcgctcaagagcctgctgtggcgcatcgcgtctccttcgatcaccagcatgcgccccttcaccgccactaccaagccggagcacgaggatcaggggctgcaggccccggcgccgccccacgcggccgcggagccggacctgctctactacccacctggcgtcgtggtctacagcggggggcgctacgacctgctgcccagcagctctgcctactgacgcaggcctcaggcccagggcgcgccgtcggggcaaggtggccttccccgggcggtaaagaggtgaaaggatgaaggagggctgggg

SEQ ID NO: 2 - protein, 469 aa, Homo sapiens
MQMADAATIATMNKAAGGDKLAELFSLVPDLLEAANTSGNASLQLPDLWWELGLELPDGAPPGHPPGSGGAESADTEARVRILISVVYWVVCALGLAGNLLVLYLMKSMQGWRKSSINLFVTNLALTDFQFVLTLPFWAVENALDFKWPFGKAMCKIVSMVTSMNMYASVFFLTAMSVTRYHSVASALKSHRTRGHGRGDCCGRSLGDSCCFSAKALCVWIWALAALASLPSAIFSTTVKVMGEELCLVRFPDKLLGRDRQFWLGLYHSQKVLLGFVLPLGIIILCYLLLVRFIADRRAAGTKGGAAVAGGRPTGASARRLSKVTKSVTIVVLSFFLCWLPNQALTTWSILIKFNAVPFSQEYFLCQVYAFPVSVCLAHSNSCLNPVLYCLVRREFRKALKSLLWRIASPSITSMRPFTATTKPEHEDQGLQAPAPPHAAAEPDLLYYPPGVVVYSGGRYDLLPSSSAY

SEQ ID NO: 3 - DNA, 429 bp, Homo sapiens
atggccaggtacatgctgctgctgctcctggcggtatgggtgctgaccggggagctgtggccgggagctgaggcccgggcagcgccttacggggtcaggctttgcggccgagaattcatccgagcagtcatcttcacctgcgggggctcccggtggagacgatcagacatcctggcccacgaggctatgggagataccttcccggatgcagatgctgatgaagacagtctggcaggcgagctggatgaggccatggggtccagcgagtggctggccctgaccaagtcaccccaggccttttacagggggcgacccagctggcaaggaacccctggggttcttcggggcagccgagatgtcctggctggcctttccagcagctgctgcaagtgggggtgtagcaaaagtgaaatcagtagcctttgctag

SEQ ID NO: 4 - protein, 142 aa, Homo sapiens
MARYMLLLLLAVWVLTGELWPGAEARAAPYGVRLCGREFIRAVIFTCGGSRWRRSDILAHEAMGDTFPDADADEDSLAGELDEAMGSSEWLALTKSPQAFYRGRPSWQGTPGVLRGSRDVLAGLSSSCCKWGCSKSEISSLC

SEQ ID NO: 5 - DNA, 29 bp, Artificial Sequence
  Description of Artificial Sequence Synthetic Oligonucleotide
gcagccacgatagccaccatgaataaggc

SEQ ID NO: 6 - RNA, 29 nt, Artificial Sequence
  Description of Artificial Sequence Synthetic Oligonucleotide
gcagccacgauagccaccaugaauaaggc

SEQ ID NO: 7 - RNA, 29 nt, Artificial Sequence
  Description of Artificial Sequence Synthetic Oligonucleotide
gccuuauucaugguggcuaucguggcugc

SEQ ID NO: 8 - DNA, 29 bp, Artificial Sequence
  Description of Artificial Sequence Synthetic Oligonucleotide
gccacgatagccaccatgaataaggcagc

SEQ ID NO: 9 - RNA, 29 nt, Artificial Sequence
  Description of Artificial Sequence Synthetic Oligonucleotide
gccacgauagccaccaugaauaaggcagc

SEQ ID NO: 10 - RNA, 29 nt, Artificial Sequence
  Description of Artificial Sequence Synthetic Oligonucleotide
gcugccuuauucaugguggcuaucguggc

SEQ ID NO: 11 - DNA, 29 bp, Artificial Sequence
  Description of Artificial Sequence Synthetic Oligonucleotide
gctgcagcttccggacttgtggtgggagc

SEQ ID NO: 12 - RNA, 29 nt, Artificial Sequence
  Description of Artificial Sequence Synthetic Oligonucleotide
gcugcagcuuccggacuuguggugggagc

SEQ ID NO: 13 - RNA, 29 nt, Artificial Sequence
  Description of Artificial Sequence Synthetic Oligonucleotide
gcucccaccacaaguccggaagcugcagc

SEQ ID NO: 14 - DNA, 29 bp, Artificial Sequence
  Description of Artificial Sequence Synthetic Oligonucleotide
gcttccggacttgtggtgggagctggggc

SEQ ID NO: 15 - RNA, 29 nt, Artificial Sequence
  Description of Artificial Sequence Synthetic Oligonucleotide
gcuuccggacuuguggugggagcuggggc

SEQ ID NO: 16 - RNA, 29 nt, Artificial Sequence
  Description of Artificial Sequence Synthetic Oligonucleotide
gccccagcucccaccacaaguccggaagc

SEQ ID NO: 17 - DNA, 29 bp, Artificial Sequence
  Description of Artificial Sequence Synthetic Oligonucleotide
ggttggcgggcaacctgctggttctctac

SEQ ID NO: 18 - RNA, 29 nt, Artificial Sequence
  Description of Artificial Sequence Synthetic Oligonucleotide
gguuggcgggcaaccugcugguucucuac

SEQ ID NO: 19 - RNA, 29 nt, Artificial Sequence
  Description of Artificial Sequence Synthetic Oligonucleotide
guagagaaccagcagguugcccgccaacc

SEQ ID NO: 20 - DNA, 29 bp, Artificial Sequence
  Description of Artificial Sequence Synthetic Oligonucleotide
gttggcgggcaacctgctggttctctacc

SEQ ID NO: 21 - RNA, 29 nt, Artificial Sequence
  Description of Artificial Sequence Synthetic Oligonucleotide
guuggcgggcaaccugcugguucucuacc

SEQ ID NO: 22 - RNA, 29 nt, Artificial Sequence
  Description of Artificial Sequence Synthetic Oligonucleotide
gguagagaaccagcagguugcccgccaac

SEQ ID NO: 23 - DNA, 29 bp, Artificial Sequence
  Description of Artificial Sequence Synthetic Oligonucleotide
gggcgctacgacctgctgcccagcagctc

SEQ ID NO: 24 - RNA, 29 nt, Artificial Sequence
  Description of Artificial Sequence Synthetic Oligonucleotide
gggcgcuacgaccugcugcccagcagcuc

SEQ ID NO: 25 - RNA, 29 nt, Artificial Sequence
  Description of Artificial Sequence Synthetic Oligonucleotide
gagcugcugggcagcaggucguagcgaaa

SEQ ID NO: 26 - DNA, 29 bp, Artificial Sequence
  Description of Artificial Sequence Synthetic Oligonucleotide
gctacgacctgctgcccagcagctctgcc

SEQ ID NO: 27 - RNA, 29 nt, Artificial Sequence
  Description of Artificial Sequence Synthetic Oligonucleotide
gcuacgaccugcugcccagcagcucugcc

SEQ ID NO: 28 - RNA, 29 nt, Artificial Sequence
  Description of Artificial Sequence Synthetic Oligonucleotide
ggcagagcugcugggcagcaggucguagc

SEQ ID NO: 29 - DNA, 6 bp, Artificial Sequence
  Description of Artificial Sequence Synthetic Oligonucleotide
aaaaaa

SEQ ID NO: 30 - DNA, 29 bp, Artificial Sequence
  Description of Artificial Sequence Synthetic Oligonucleotide
gccttattcatggtggctatcgtggctgc

SEQ ID NO: 31 - DNA, 8 bp, Artificial Sequence
  Description of Artificial Sequence Synthetic Oligonucleotide
caagcttc

SEQ ID NO: 32 - DNA, 21 bp, Artificial Sequence
  Description of Artificial Sequence Synthetic Oligonucleotide
ggtgtttcgtcctttccacaa

SEQ ID NO: 33 - RNA, 68 nt, Artificial Sequence
  Description of Artificial Sequence Synthetic Oligonucleotide
gcagccacgauagccaccaugaauaaggcgaagcuuggccuuauucaugguggcuaucguggcugcuu

SEQ ID NO: 34 - RNA, 72 nt, Artificial Sequence
  Description of Artificial Sequence Synthetic Oligonucleotide
gcagccacgauagccaccaugaauaaggcgaagcuuggccuuauucaugguggcuaucguggcugcuuuuuu

SEQ ID NO: 35 - RNA, 72 nt, Artificial Sequence
  Description of Artificial Sequence Synthetic Oligonucleotide
aaaaaagcagccacgauagccaccaugaauaaggccaagcuucgccuuauucaugguggcuaucguggcugc

SEQ ID NO: 36 - RNA, 21 nt, Artificial Sequence
  Description of Artificial Sequence Synthetic Oligonucleotide
gguguuucguccuuuccacaa

SEQ ID NO: 37 - RNA, 93 nt, Artificial Sequence
  Description of Artificial Sequence Synthetic Oligonucleotide
aaaaaagcagccacgauagccaccaugaauaaggccaagcuucgccuuauucaugguggcuaucguggcugcgguguuucguccuuuccacaa

SEQ ID NO: 38 - RNA, 93 nt, Artificial Sequence; identical to SEQ ID NO: 37
  Description of Artificial Sequence Synthetic Oligonucleotide

SEQ ID NO: 39 - RNA, 93 nt, Artificial Sequence
  Description of Artificial Sequence Synthetic Oligonucleotide
aaaaaagccacgauagccaccaugaauaaggcagccaagcuuccggugcuaucggugguacuuauuccgucggguguuucguccuuuccacaa

SEQ ID NO: 40 - RNA, 93 nt, Artificial Sequence
  Description of Artificial Sequence Synthetic Oligonucleotide
aaaaaagggcgcuacgaccugcugcccagcagcuccaagcuucgagcugcugggcagcaggucguagcgcccgguguuucguccuuuccacaa

SEQ ID NO: 41 - RNA, 93 nt, Artificial Sequence
  Description of Artificial Sequence Synthetic Oligonucleotide
aaaaaagcuacgaccugcugcccagcagcucugcccaagcuucggcagagcugcugggcagcaggucguagcgguguuucguccuuuccacaa

SEQ ID NO: 42 - DNA, 29 bp, Artificial Sequence
  Description of Artificial Sequence Synthetic Oligonucleotide
gccaggtacatgctgctgctgctcctggc

SEQ ID NO: 43 - RNA, 29 nt, Artificial Sequence
  Description of Artificial Sequence Synthetic Oligonucleotide
gccagguacaugcugcugcugcuccuggc

SEQ ID NO: 44 - RNA, 29 nt, Artificial Sequence
  Description of Artificial Sequence Synthetic Oligonucleotide
gccaggagcagcagcagcauguaccuggc

SEQ ID NO: 45 - DNA, 29 bp, Artificial Sequence
  Description of Artificial Sequence Synthetic Oligonucleotide
gggcagcgccttacggggtcaggctttgc

SEQ ID NO: 46 - RNA, 29 nt, Artificial Sequence
  Description of Artificial Sequence Synthetic Oligonucleotide
gggcagcgccuuacggggucaggcuuugc

SEQ ID NO: 47 - RNA, 29 nt, Artificial Sequence
  Description of Artificial Sequence Synthetic Oligonucleotide
gcaaagccugaccccguaaggcgcugccc

SEQ ID NO: 48 - DNA, 29 bp, Artificial Sequence
  Description of Artificial Sequence Synthetic Oligonucleotide
gagcagtcatcttcacctgcgggggctcc

SEQ ID NO: 49 - RNA, 29 nt, Artificial Sequence
  Description of Artificial Sequence Synthetic Oligonucleotide
gagcagucaucuucaccugcgggggcucc

SEQ ID NO: 50 - RNA, 29 nt, Artificial Sequence
  Description of Artificial Sequence Synthetic Oligonucleotide
ggagcccccgcaggugaagaugacugcuc

SEQ ID NO: 51 - DNA, 29 bp, Artificial Sequence
  Description of Artificial Sequence Synthetic Oligonucleotide
gtagcaaaagtgaaatcagtagcctttgc

SEQ ID NO: 52 - RNA, 29 nt, Artificial Sequence
  Description of Artificial Sequence Synthetic Oligonucleotide
guagcaaaagugaaaucaguagccuuugc

SEQ ID NO: 53 - RNA, 29 nt, Artificial Sequence
  Description of Artificial Sequence Synthetic Oligonucleotide
cgaaaggcuacugauuucacuuuugcuac

SEQ ID NO: 54 - DNA, 29 bp, Artificial Sequence
  Description of Artificial Sequence Synthetic Oligonucleotide
gccaggagcagcagcagcatgtacctggc

SEQ ID NO: 55 - RNA, 68 nt, Artificial Sequence
  Description of Artificial Sequence Synthetic Oligonucleotide
gccaggagcagcagcagcauguaccuggcgaagcuuggccagguacaugcugcugcugcuccuggcuu

SEQ ID NO: 56 - RNA, 72 nt, Artificial Sequence
  Description of Artificial Sequence Synthetic Oligonucleotide
gccaggagcagcagcagcauguaccuggcgaagcuuggccagguacaugcugcugcugcuccuggcuuuuuu

SEQ ID NO: 57 - RNA, 72 nt, Artificial Sequence
  Description of Artificial Sequence Synthetic Oligonucleotide
aaaaaacgguccucgucgucgucguacauggaccgcaagcuucgccaggagcagcagcagcauguaccuggc

SEQ ID NO: 58 - RNA, 93 nt, Artificial Sequence
  Description of Artificial Sequence Synthetic Oligonucleotide
aaaaaacgguccucgucgucgucguacauggaccgcaagcuucgccaggagcagcagcagcauguaccuggcgguguuucguccuuuccacaa

SEQ ID NO: 59 - RNA, 93 nt, Artificial Sequence
  Description of Artificial Sequence Synthetic Oligonucleotide
aaaaaagccagguacaugcugcugcugcuccuggccaagcuucgccaggagcagcagcagcauguaccuggcgguguuucguccuuuccacaa

SEQ ID NO: 60 - RNA, 93 nt, Artificial Sequence
  Description of Artificial Sequence Synthetic Oligonucleotide
aaaaaagggcagcgccuuacggggucaggcuuugccaagcuucgcaaagccugaccccguaaggcgcugcccgguguuucguccuuuccacaa

SEQ ID NO: 61 - RNA, 93 nt, Artificial Sequence
  Description of Artificial Sequence Synthetic Oligonucleotide
aaaaaagagcagucaucuucaccugcgggggcucccaagcuucggagcccccgcaggugaagaugacugcucgguguuucguccuuuccacaa

SEQ ID NO: 62 - RNA, 93 nt, Artificial Sequence
  Description of Artificial Sequence Synthetic Oligonucleotide
aaaaaaguagcaaaagugaaaucaguagccuuugccaagcuuccgaaaggcuacugauuucacuuuugcuacgguguuucguccuuuccacaa

SEQ ID NO: 63 - DNA, 31 bp, Artificial Sequence
  Description of Artificial Sequence Synthetic Oligonucleotide
aannnnnnnnnnnnnnnnnnnnnnnnnnntt

(19) United States
(12) Patent Application Publication
(10) Pub. No.: US 2005/0026194 A1
(43) Pub. Date: Feb. 3, 2005

(54) Gene amplification and overexpression in cancer

(75) Inventor: David Mu, Jericho, NY (US)

Correspondence Address: HELLER EHRMAN WHITE & MCAULIFFE LLP, 1666 K STREET,NW, SUITE 300, WASHINGTON, DC, 20006, US

(73) Assignee: Tularik Inc., South San Francisco, CA (US)

(21) Appl. No.: 10/867,183

(22) Filed: Jun. 15, 2004

Related U.S. Application Data
(60) Provisional application No. 60/479,833, filed on Jun. 20, 2003.

(51) Int. Cl. C12Q 1/68; C12P 19/34; A61K 48/00

(52) U.S. Cl. 435/6; 435/91.2; 514/44

(57) ABSTRACT

There are disclosed methods and compositions for the diagnosis, prevention, and treatment of tumors and cancers in mammals, for example, humans, utilizing a gene, which is amplified in many types of cancer. The amplified genes, their expressed protein products and antibodies are used diagnostically or as targets for cancer therapy or as vaccines; they also are used to identify compounds and reagents useful in cancer diagnosis, prevention, and therapy.

This application claims priority to U.S. Ser. No. 60/479,833, filed Jun. 20, 2003, the entirety of which is hereby incorporated by reference.

### BACKGROUND OF THE INVENTION

1. Field of the Invention

The invention relates to oncogenes and to cancer diagnostics and therapeutics. More specifically, the present invention relates to amplified and/or overexpressed Somatostatin- and Angiotensin-Like Peptide Receptor (SALPR) and Relaxin-3 genes, each of which are involved in certain types of cancers. The invention pertains to the amplified genes, their encoded proteins, and antibodies, inhibitors, activators and the like and their use in cancer diagnostics, vaccines, and anti-cancer therapy.

2. Background of the Invention

Cancer and Gene Amplification:

Cancer is the second leading cause of death in the United States, after heart disease (Boring, et al., CA Cancer J. Clin., 43:7, 1993), and it develops in one in three Americans. One of every four Americans dies of cancer. Cancer features uncontrolled cellular growth, which results either in local invasion of normal tissue or systemic spread of the abnormal growth. A particular type of cancer or a particular stage of cancer development may involve both elements.

The division or growth of cells in various tissues functioning in a living body normally takes place in an orderly and controlled manner. This is enabled by a delicate growth control mechanism, which involves, among other things, contact, signaling, and other communication between neighboring cells. Growth signals, stimulatory or inhibitory, are routinely exchanged between cells in a functioning tissue. Cells normally do not divide in the absence of stimulatory signals, and will cease dividing when dominated by inhibitory signals. However, such signaling or communication becomes defective or completely breaks down in cancer cells. As a result, the cells continue to divide; they invade adjacent structures, break away from the original tumor mass, and establish new growth in other parts of the body. The latter progression to malignancy is referred to as “metastasis.”

Cancer generally refers to malignant tumors, rather than benign tumors. Benign tumor cells are similar to normal, surrounding cells. These types of tumors are almost always encapsulated in a fibrous capsule and do not have the potential to metastasize to other parts of the body. These tumors affect local organs but do not destroy them; they usually remain small without producing symptoms for many years. Treatment becomes necessary only when the tumors grow large enough to interfere with other organs. Malignant tumors, by contrast, grow faster than benign tumors, and they penetrate and destroy local tissues. Some malignant tumors may spread throughout the body via blood or the lymphatic system. The unpredictable and uncontrolled growth makes malignant cancers dangerous, and fatal in many cases. These tumors are not morphologically typical of the original tissue and are not encapsulated. Malignant tumors commonly recur after surgical removal.

Accordingly, treatment ordinarily is directed towards malignant cancers or malignant tumors. The intervention of malignant growth is most effective at the early stage of the cancer development. Thus, it can be important to discover sensitive markers for early signs of cancer formation and to identify potent growth suppression agents associated therewith. The development of such diagnostic and therapeutic agents involves an understanding of the genetic control mechanisms for cell division and differentiation, particularly in connection with tumorigenesis.

Cancer can be caused by inherited or acquired mutations in cancer genes, which have normal cellular functions and which induce or otherwise contribute to cancer once mutated or expressed at an abnormal level. Certain well-studied tumors carry several different independently mutated genes, including activated oncogenes and inactivated tumor suppressor genes. Each of these mutations appears to be responsible for imparting some of the traits that, in aggregate, represent the full neoplastic phenotype (Land et al., Science, 222:771, 1983; Ruley, Nature, 4:602, 1983; Hunter, Cell, 64:249, 1991).

One such mutation is gene amplification. Gene amplification involves a chromosomal region bearing specific genes undergoing a relative increase in DNA copy number, thereby increasing the copies of any genes that are present. In general, gene amplification often results in increased levels of transcription and translation, producing higher amounts of the corresponding gene mRNA and protein. Amplification of genes can cause deleterious effects, which contribute to cancer formation and proliferation (Lengauer et al. Nature, 396:643-649,1999).

It is commonly appreciated by cancer researchers that whole collections of genes are demonstrably overexpressed or differentially expressed in a variety of different types of tumor cells. Yet, often only a very small number of these overexpressed genes are likely to be causally involved in the cancer phenotype. The remaining overexpressed genes likely are secondary consequences of more basic primary events, for example, overexpression of a cluster of genes, involved in DNA replication. Nevertheless, gene amplification is established as an important genetic alteration in solid tumors (Knuutila et al., Am. J. Pathol., 152(5):1107-23, 1998; Knuutila et al., Cancer Genet. Cytogenet., 100(1):25-30, 1998).

The overexpression of certain well known genes, for example, c-myc, has been observed at fairly high levels in the absence of gene amplification (Yoshimoto et al., JPN J. Cancer Res., 77(6):540-5, 1986), although these genes are frequently amplified (Knuutila et al., Am. J. Pathol., 152(5):1107-23, 1998) and thereby activated. Such a characteristic is considered a hallmark of oncogenes. Overexpression in the absence of amplification may be caused by higher transcription efficiency in those situations. In the case of c-myc, for example, Yoshimoto et al. showed that its transcriptional rate was greatly increased in the tested tumor cell lines. The characteristics and interplay of overexpression and amplification of a gene in cancer tissues, therefore, provide significant indications of the gene's role in cancer development. That is, increased DNA copies of certain genes in tumors, along with and beyond their overexpression, may point to their functions in tumor formation and progression.

It must be remembered that overexpression and amplification are not the same phenomenon. Overexpression can be obtained from a single, unamplified gene, and an amplified gene does not always lead to greater expression levels of mRNA and protein. Thus, it is not possible to predict whether one phenomenon will result in, or is related to, the other. However, in situations where both amplification of a gene and overexpression of the gene product occur in cells or tissues that are in a precancerous or cancerous state, then that gene and its product present both a diagnostic target and a therapeutic opportunity for intervention. Amplification, without overexpression, and overexpression, without amplification, also can be correlated with and indicative of cancers and pre-cancers.

Because some genes are sometimes amplified as a consequence of their location next to a true oncogene, it also is beneficial to determine the DNA copy number of nearby genes in a panel of tumors so that amplified genes that are in the epicenter of the amplification unit can be distinguished from amplified genes that are occasionally amplified due to their proximity to another, more relevant, amplified gene.

Thus, discovery and characterization of amplified cancer genes, along with and in addition to their features of overexpression or differential expression, will be a promising avenue that leads to novel targets for diagnostic, vaccines, and therapeutic applications.

Additionally, the completion of the working drafts of the human genome and the paralleled advances in genomics technologies offer new promises in the identification of effective cancer markers and the anti-cancer agents. The high-throughput microarray detection and screening technology, computer-empowered genetics and genomics analysis tools, and multi-platform functional genomics and proteomics validation systems, all assist in applications in cancer research and findings. With the advent of modern sequencing technologies and genomic analyses, many unknown genes and genes with unknown or partially known functions can be revealed.

Genomic amplification and overexpression of Homo sapiens Somatostatin- and Angiotensin-Like Peptide Receptor (SALPR) and Relaxin-3 (H3) (RLN3) genes and their role in tumorogenesis were not known until the instant invention. In addition to antibodies that bind tumor cells expressing SALPR or Relaxin-3, the possibility to treat tumors with antibodies that block the oncogenic function of SALPR or Relaxin-3, and thereby mediate tumor-cell killing, were not known until the present invention.

Therefore, there is a need in the art for an understanding of SALPR and Relaxin-3 genes regulation. Understanding the physiological role of human SALPR and Relaxin-3 genes will facilitate early diagnosis of abnormalities associated therewith and lead to appropriate therapies to treat such abnormalities. These needs are satisfied for the first time by the present invention.

### SUMMARY OF THE INVENTION

The present invention relates to isolation, characterization, overexpression and implication of genes, including amplified genes, in cancers, methods and compositions for use in diagnosis, vaccines, prevention and treatment of tumors and cancers, for example, lung cancer, colon cancer, ovarian cancer, and pancreatic cancer, in mammals, for example, humans. The invention is based on the finding of novel attributes of SALPR and Relaxin-3. Specifically, amplification and/or overexpression of SALPR and/or Relaxin-3 genes in tumors, including lung tumors, colon tumors, ovarian tumors, and pancreatic tumors, and their role in oncogenesis was not known until the instant invention.

These novel attributes include the overexpression of the SALPR and/or Relaxin-3 genes in certain cancers, for example, lung cancer and/or colon cancer and/or ovarian cancer and/or pancreatic cancer, and the frequent amplification of SALPR and/or Relaxin-3 in cancer cells. The SALPR and/or Relaxin-3 genes and their expressed protein products can thus be used diagnostically or as targets for cancer therapy; and they also can be used to identify and design compounds useful in the diagnosis, prevention, and therapy of tumors and cancers.

Until the present invention, certain utilities of the SALPR and Relaxin-3 genes associated with diagnostics and therapeutics in various cancers were not known. Moreover, until the present invention, SALPR and Relaxin-3 genes have not been fully characterized to allow their role in tumor development to be completely understood.

According to one aspect of the present invention, the use of SALPR and/or Relaxin-3 in gene therapy, development of small molecule inhibitors, small interfering RNAs (siRNAs), microRNAs (miRNAs), and antisense nucleic acids, and development of immunodiagnostics and immunotherapies, are provided. The present invention includes production and the use of antibodies, for example, monoclonal, polyclonal, single-chain and engineered antibodies (including humanized antibodies) and fragments, which specifically bind SALPR and/or Relaxin-3 proteins and polypeptides. The invention also includes antagonists and inhibitors of SALPR and Relaxin-3 proteins that can inhibit one or more of the functions or activities of SALPR or Relaxin-3, respectively. Suitable antagonists can include small molecules (molecular weight below about 500 Daltons), large molecules (molecular weight above about 500 Daltons), and antibodies (including fragments and single chain antibodies) that bind and interfere or neutralize SALPR or Relaxin-3 proteins, polypeptides which compete with a native form of SALPR or Relaxin-3 proteins for binding to a protein that naturally interacts with SALPR or Relaxin-3 proteins, and nucleic acid molecules that interfere with transcription and/or translation of the SALPR or Relaxin-3 gene (for example, antisense nucleic acid molecules, triple helix forming molecules, ribozymes, microRNAs, and small interfering RNAs), respectively. The present invention also includes useful compounds that influence or attenuate activities of SALPR or Relaxin-3.

In addition, the present invention provides inhibitors of SALPR and Relaxin-3 activity, such as antibodies, that block the oncogenic function or anti-apoptotic activity of SALPR and Relaxin-3, respectively.

Other inhibitors include antibodies that bind to a cell over-expressing SALPR or Relaxin-3 protein, thereby resulting in suppression or death of the cell.

The present invention further provides molecules that can decrease the expression of SALPR or Relaxin-3 by affecting transcription or translation. Small molecules (molecular weight below about 500 Daltons), large molecules (molecular weight above about 500 Daltons), and nucleic acid molecules, for example, ribozymes, miRNAs, siRNAs and antisense molecules, including antisense RNA, antisense DNA or decoy molecules (for example, Morishita et al., Ann. N Y Acad. Sci., 947:294-301, 2001; Andratschke et al., Anticancer Res., 21:(5)3541-3550, 2001), may all be utilized to inhibit the expression or amplification.

As mentioned above, the SALPR and Relaxin-3 gene sequences also can be employed in an RNA interference context. The phenomenon of RNA interference is described and discussed in Bass, Nature, 411: 428-29 (2001); Elbashir et al., Nature 411: 494-98 (2001); and Fire et al., Nature, 391: 806-11 (1998), where methods of making interfering RNA also are discussed.

In one aspect, the present invention provides methods for diagnosing or predicting a cancer (diagnostics or predictive uses) for example, a lung cancer, a colon cancer, an ovarian cancer, or a pancreatic cancer, in a mammal, which comprises, in any practical order, obtaining a test sample from a region in the tissue that is suspected to be precancerous or cancerous; and comparing the average number of SALPR or Relaxin-3 gene copies measured (for example, quantitatively and/or qualitatively) in the sample to a control sample or a known value, thereby determining whether the SALPR or Relaxin-3 genes are amplified in the test sample, respectively, wherein amplification of the SALPR or Relaxin-3 gene indicates a cancer or a precancerous condition in the tissue.

In another aspect, the present invention provides methods for diagnosing or predicting a cancer (diagnostics or predictive uses) for example, a lung cancer, a colon cancer, an ovarian cancer, or a pancreatic cancer, in a mammal, which comprises, in any practical order, obtaining a test sample from a region in the tissue that is suspected to be precancerous or cancerous; obtaining a control sample from a region in the tissue or other tissues that are normal; and detecting or measuring in both the test sample and the control sample the level of SALPR or Relaxin-3 mRNA transcripts, wherein a level of the transcripts higher in the test sample than that in the control sample indicates a cancer or a precancerous condition in the tissue. In another aspect the control sample may be obtained from a different individual or be a normalized value based on baseline data obtained from a population.

In another aspect, the present invention provides methods for diagnosing or predicting a cancer (diagnostics or predictive uses) for example, a lung cancer, a colon cancer, an ovarian cancer, or a pancreatic cancer, in a mammal, which comprises, in any practical order, obtaining a test sample from a region in the tissue that is suspected to be precancerous or cancerous; and comparing the average number of SALPR or Relaxin-3 DNA copies detected (for example, quantitatively and/or qualitatively) in the sample to a control sample or a known value, thereby determining whether the SALPR or Relaxin-3 genes are amplified in the test sample, respectively, wherein amplification of the SALPR or Relaxin-3 gene indicates a cancer or a precancerous condition in the tissue.

Another aspect of the present invention provides methods for diagnosing or predicting a cancer (diagnostics or predictive uses) for example, a lung cancer, a colon cancer, an ovarian cancer, or a pancreatic cancer, in a mammal, which comprises, in any practical order, obtaining a test sample from a region in the tissue that is suspected to be precancerous or cancerous; contacting the sample with anti-SALPR or anti-Relaxin-3 antibodies, and detecting in the test sample, the level of SALPR or Relaxin-3 expression, respectively, wherein an increased level of the SALPR or Relaxin-3 expression in the test sample, as compared to a control sample or a known value indicates a precancerous or a cancerous condition in the tissue. In another aspect, the control sample may be obtained from a different individual or be a normalized value based on baseline data obtained from a population. Alternatively, a given level of SALPR or Relaxin-3, representative of the cancer-free population, that has been previously established based on measurements from normal, cancer-free animals, can be used as a control. A control data point from a reference database, based on data obtained from control samples representative of a cancer-free population, also can be used as a control.

In another aspect, the present invention relates to methods for comparing and compiling data wherein the data is stored in electronic or paper format. Electronic format can be selected from the group consisting of electronic mail, disk, compact disk (CD), digital versatile disk (DVD), memory card, memory chip, ROM or RAM, magnetic optical disk, tape, video, video clip, microfilm, internet, shared network, shared server and the like; wherein data is displayed, transmitted or analyzed via electronic transmission, video display, telecommunication, or by using any of the above stored formats; wherein data is compared and compiled at the site of sampling specimens or at a location where the data is transported following a process as described above.

In another aspect, the present invention provides methods for preventing, controlling, reversing, or suppressing cancer growth (and analogous uses) in a mammalian organ and tissue, for example, in the lung, colon, ovary, or pancreas, which comprises administering an inhibitor of SALPR or Relaxin-3 protein to the organ or tissue, thereby inhibiting SALPR or Relaxin-3 protein activities, respectively. Such inhibitors may be, among other things, an antibody to SALPR or Relaxin-3 protein or polypeptide portions thereof, an antagonist to SALPR or Relaxin-3 protein, respectively, or other small or large molecules.

In a further aspect, the present invention provides a method for preventing, controlling, reversing, or suppressing cancer growth (and analogous uses) in a mammalian organ and tissue, for example, in the lung, colon, ovary, or pancreas, which comprises administering to the organ or tissue a nucleotide molecule that is capable of interacting with SALPR or Relaxin-3 DNA and/or RNA and thereby blocking or interfering the SALPR or Relaxin-3 gene functions, respectively. Such nucleotide molecules can be an antisense nucleotide of the SALPR or Relaxin-3 gene, a ribozyme of SALPR or Relaxin-3 RNA, a small interfering RNA (siRNA) or it may be a molecule capable of forming a triple helix with the SALPR or Relaxin-3 gene, respectively.

In a further aspect, the present invention provides methods for preventing, controlling, reversing, or suppressing cancer growth (and analogous uses) in a mammalian organ and tissue, for example, in the lung, colon, ovary, or pancreas, which comprises administering to the organ or tissue a nucleotide molecule that is capable of interacting with SALPR or Relaxin-3 DNA and/or RNA and thereby blocking or interfering the SALPR or Relaxin-3 gene function, respectively. Such nucleotide molecules can be an antisense nucleotide of the SALPR or Relaxin-3 gene, a ribozyme of SALPR or Relaxin-3 RNA; a small interfering RNA; a microRNA (miRNA); or it may be a molecule capable of forming a triple helix with the SALPR or Relaxin-3 gene, respectively.

In still a further aspect, the present invention provides methods for determining the efficacy, such as potency, of a therapeutic treatment regimen for treating a cancer (and analogous uses), for example, a lung cancer, a colon cancer, an ovarian cancer, or a pancreatic cancer, in a patient, for example, in a clinical trial or other research studies, which comprises, in any practical order, obtaining a first sample from the patient to ultimately obtain a pre-treatment level; administering the treatment regimen to the patient; obtaining a second sample from the patient after a time period to ultimately obtain a test level; and detecting in both the first and the second samples the level of SALPR or Relaxin-3 mRNA transcripts, wherein a level of the transcripts lower in the second sample (test level) than that in the first sample (pre-treatment level) indicates that the treatment regimen is effective in the patient.

In another aspect, the present invention provides methods for determining the efficacy, such as potency, of a compound to suppress a cancer (and analogous uses), for example, a lung cancer, a colon cancer, an ovarian cancer, or a pancreatic cancer, in a patient, for example, in a clinical trial or other research studies, which comprises, in any practical order, obtaining a first sample from the patient to ultimately obtain a pre-treatment level; administering the treatment regimen to the patient; obtaining the second sample from the patient after a time period to ultimately obtain a test level; and detecting in both the first and the second samples the level of SALPR or Relaxin-3 mRNA transcripts, wherein a level of the transcripts lower in the second sample (test level) than that in the first sample (pre-treatment level) indicates that the compound is effective to suppress such a cancer or a precancerous condition.

In another aspect, the present invention provides methods for determining the efficacy, such as potency, of a therapeutic treatment regimen for treating a cancer (and analogous uses), for example, a lung cancer, a colon cancer, an ovarian cancer, or a pancreatic cancer, in a patient, for example, in a clinical trial or other research studies, which comprises, in any practical order, obtaining a first sample from the patient to ultimately obtain a pre-treatment level; administering the treatment regimen to the patient; obtaining a second sample from the patient after a time period to ultimately obtain a test level; and detecting in both the first and the second samples the average number of SALPR or Relaxin-3 DNA copies per cell, for example, thereby determining the overall or average SALPR or Relaxin-3 gene amplification state in the first and second samples, respectively, wherein a lower number of SALPR or Relaxin-3 DNA copies per cell, or average, for example, in the second sample (test level) than that in the first sample (pre-treatment level) indicates that the treatment regimen is effective.

In yet another aspect, the present invention provides methods for determining the efficacy, such as potency, of a therapeutic treatment regimen for treating a cancer (and analogous uses), for example, a lung cancer, a colon cancer, an ovarian cancer, or a pancreatic cancer, in a patient, which comprises, in any practical order, obtaining a first sample from the patient to ultimately obtain a pre-treatment level; administering the treatment regimen to the patient; obtaining a second sample from the patient after a time period to ultimately obtain a test level; contacting the samples with anti-SALPR or anti-Relaxin-3 antibodies, and detecting the level of SALPR or Relaxin-3 expression in both the first and the second samples, respectively. A lower level of the SALPR or Relaxin-3 expression in the second sample (test level) than that in the first sample (pre-treatment level) indicates that the treatment regimen is effective in the patient.

Yet, in another aspect, the invention provides methods for determining the efficacy, such as potency, of a therapeutic treatment regimen for treating a cancer (and analogous uses), for example, a lung cancer, a colon cancer, an ovarian cancer, or a pancreatic cancer, in a patient, comprising, in any practical order, the steps of: obtaining a first sample from the patient to ultimately obtain a pre-treatment level; administering the treatment regimen to the patient; obtaining a second sample from the patient after a time period to ultimately obtain a test level; contacting the samples with anti-SALPR or anti-Relaxin-3 antibodies, determining the expression level of SALPR or Relaxin-3, in both the first and the second samples, by determining the overall expression divided by the number of cells present in each sample; and comparing the expression level of SALPR or Relaxin-3 in the first and the second samples, respectively. A lower level of the SALPR or Relaxin-3 expression in the second sample (test level) than that in the first sample (pre-treatment level) indicates that the treatment regimen is effective in the patient, wherein the expression level is determined via a binding assay or other appropriate assays, including reverse transcription and polymerase chain reaction (RT-PCR), Northern hybridization, microarray analysis, enzyme immuno assay (EIA), two-hybrid assays such as GAL4 DNA binding domain based assays, blot assays, sandwich assays, and the like.

In still another aspect, the present invention provides methods for determining the efficacy, such as potency, of a compound to suppress a cancer (and analogous uses), for example, a lung cancer, a colon cancer, an ovarian cancer, or a pancreatic cancer, in a patient, for example, in a clinical trial or other research studies, which comprises, in any practical order, obtaining a first sample from the patient to ultimately obtain a pre-treatment level; administering the treatment regimen to the patient; obtaining a second sample from the patient after a time period to ultimately obtain a test level; and detecting in both the first and the second samples the average number of SALPR or Relaxin-3 DNA copies per cell, for example, thereby determining the SALPR or Relaxin-3 gene amplification state in the first and second samples, respectively, wherein a lower number of SALPR or Relaxin-3 DNA copies per cell, or average, for example, in the second sample (test level) than that in the first sample (pre-treatment level) indicates that the compound is effective.

In another aspect, the present invention provides methods for monitoring the efficacy, such as potency, of a therapeutic treatment regimen for treating a cancer (and analogous uses), for example, a lung cancer, a colon cancer, an ovarian cancer, or a pancreatic cancer, in a patient, for example, in a clinical trial or other research studies, which comprises, in any practical order, obtaining a first sample from the patient to ultimately obtain a pre-treatment level; administering the treatment regimen to the patient; obtaining a second sample from the patient after a time period to ultimately obtain a test level; and detecting in both the first and the second samples the level of SALPR or Relaxin-3 mRNA transcripts, wherein a level of the transcripts lower in the second sample (test level) than that in the first sample (pre-treatment level) indicates that the treatment regimen is effective in the patient.

Yet, in another aspect, the invention provides methods for monitoring the efficacy, such as potency, of a therapeutic treatment regimen for treating a cancer (and analogous uses), for example, a lung cancer, a colon cancer, an ovarian cancer, or a pancreatic cancer, in a patient, for example, in a clinical trial or sample from the patient to ultimately obtain a pre-treatment level; administering the treatment regimen to the patient; obtaining a second sample from the patient after a time period to ultimately obtain a test level; determining in both the first and the second samples the level of SALPR or Relaxin-3 mRNA transcripts, by determining the overall level divided by the number of cells present in each sample; and comparing the level of SALPR or Relaxin-3 in the first and the second samples, respectively. A lower level of the SALPR or Relaxin-3 mRNA transcripts in the second sample (test level) than that in the first sample (pre-treatment level) indicates that the treatment regimen is effective in the patient, wherein the level can be determined via a binding assay or other appropriate assays, including RT-PCR, Northern hybridization, microarray analysis, two-hybrid assays such as GAL4 DNA binding domain based assays, blot assays, sandwich assays, and the like.

In another aspect, the present invention provides methods for monitoring the efficacy, such as potency, of a compound to suppress a cancer (and analogous uses), for example, a lung cancer, a colon cancer, an ovarian cancer, or a pancreatic cancer, in a patient, for example, in a clinical trial or other research studies, which comprises, in any practical order, obtaining a first sample from the patient to ultimately obtain a pre-treatment level; administering the treatment regimen to the patient; obtaining the second sample from the patient after a time period to ultimately obtain a test level; and detecting in both the first and the second samples the level of SALPR or Relaxin-3 mRNA transcripts, wherein a level of the transcripts lower in the second sample (test level) than that in the first sample (pre-treatment level) indicates that the compound is effective to suppress such a cancer or a precancerous condition.

In another aspect, the present invention provides methods for monitoring the efficacy, such as potency, of a therapeutic treatment regimen for treating a cancer (and analogous uses), for example, a lung cancer, a colon cancer, an ovarian cancer, or a pancreatic cancer, in a patient, for example, in a clinical trial or other research studies, which comprises, in any practical order, obtaining a first sample from the patient to ultimately obtain a pre-treatment level; administering the treatment regimen to the patient; obtaining a second sample from the patient after a time period to ultimately obtain a test level; and detecting in both the first and the second samples the average number of SALPR or Relaxin-3 DNA copies per cell, for example, thereby determining the overall or average SALPR or Relaxin-3 gene amplification state in the first and second samples, respectively, wherein a lower number of SALPR or Relaxin-3 DNA copies per cell, or average, for example, in the second sample (test level) than that in the first sample (pre-treatment level) indicates that the treatment regimen is effective.

In yet another aspect, the present invention provides methods for monitoring the efficacy, such as potency, of a therapeutic treatment regimen for treating a cancer (and analogous uses), for example, a lung cancer, a colon cancer, an ovarian cancer, or a pancreatic cancer, in a patient, which comprises, in any practical order, obtaining a first sample from the patient to ultimately obtain a pre-treatment level; administering the treatment regimen to the patient; obtaining a second sample from the patient after a time period to ultimately obtain a test level; contacting the samples with anti-SALPR or anti-Relaxin-3 antibodies, and detecting the level of SALPR or Relaxin-3 expression in both the first and the second samples, respectively. A lower level of the SALPR or Relaxin-3 expression in the second sample (test level) than in the first sample (pre-treatment level) indicates that the treatment regimen is effective in the patient.

Yet, in another aspect, the invention provides methods for monitoring the efficacy, such as potency, of a therapeutic treatment regimen for treating a cancer (and analogous uses), for example, a lung cancer, a colon cancer, an ovarian cancer, or a pancreatic cancer, in a patient, comprising, in any practical order, the steps of: obtaining a first sample from the patient to ultimately obtain a pre-treatment level; administering the treatment regimen to the patient; obtaining a second sample from the patient after a time period to ultimately obtain a test level; contacting the samples with anti-SALPR or anti-Relaxin-3 antibodies, determining the level of SALPR or Relaxin-3 expression in both the first and the second samples, by determining the overall expression divided by the number of cells present in each sample; and comparing the expression level of SALPR or Relaxin-3 in the first and the second samples, respectively. A lower level of the SALPR or Relaxin-3 expression in the second sample (test level) than that in the first sample (pre-treatment level) indicates that the treatment regimen is effective in the patient, wherein the expression level can be determined via a binding assay or other appropriate assays, including RT-PCR, Northern hybridization, microarray analysis, two-hybrid assays such as GAL4 DNA binding domain based assays, EIA, blot assays, sandwich assays, and the like.

In still another aspect, the present invention provides methods for monitoring the efficacy, such as potency, of a compound to suppress a cancer (and analogous uses), for example, a lung cancer, a colon cancer, an ovarian cancer, or a pancreatic cancer, in a patient, for example, in a clinical trial or other research studies, which comprises, in any practical order, obtaining a first sample from the patient to ultimately obtain a pre-treatment level; administering the treatment regimen to the patient; obtaining a second sample from the patient after a time period to ultimately obtain a test level; and detecting in both the first and the second samples the average number of SALPR or Relaxin-3 DNA copies per cell, for example, thereby determining the SALPR or Relaxin-3 gene amplification state in the first and second samples, respectively, wherein a lower number of SALPR or Relaxin-3 DNA copies per cell, or average, for example, in the second sample. (test level) than that in the first sample (pre-treatment level) indicates that the compound is effective.

One aspect of the invention provides methods for diagnosing or predicting cancer or cancer potential and/or monitoring the efficacy, such as potency, of a cancer therapy by using an isolated SALPR or Relaxin-3 gene amplicon, wherein the methods further comprise, in any practical order, obtaining a test sample from a region in the tissue that is suspected to be precancerous or cancerous; obtaining a control sample from a region in the tissue or other tissues that is normal; and detecting in both the test sample and the control sample the presence and extent of SALPR or Relaxin-3 gene amplicons, respectively, wherein a level of amplification higher in the test sample than that in the control sample indicates a precancerous or cancerous condition in the tissue. In one aspect, a control sample can be obtained from a biological subject representative of healthy, cancer-free animals. In another aspect, the control may be obtained from a different individual or be a normalized value based on baseline data obtained from a population.

Another aspect of the invention is to provide an isolated SALPR or Relaxin-3 gene amplicon, wherein the amplicon comprises a completely or partially amplified product of SALPR or Relaxin-3 gene, respectively, including a polynucleotide having at least about 90% sequence identity to SALPR or Relaxin-3 gene, for example, SEQ ID NO:1 (SALPR) or SEQ ID NO:3 (Relaxin-3), a polynucleotide encoding the polypeptide set forth in SEQ ID NO:2 (SALPR) or SEQ ID NO:4 (Relaxin-3) or a polynucleotide that is overexpressed in tumor cells having at least about 90% sequence identity to the polynucleotide of SEQ ID NO:1 (SALPR) or SEQ ID NO:3 (Relaxin-3) or the polynucleotide encoding the polypeptide set forth in SEQ ID NO:2 (SALPR) or SEQ ID NO:4 (Relaxin-3).

In yet another aspect, the present invention provides methods for modulating SALPR or Relaxin-3 activities by contacting a biological subject from a region that is suspected to be precancerous or cancerous with a modulator of the SALPR or Relaxin-3 protein, wherein the modulator is, for example, a small molecule.

In still another aspect, the present invention provides methods for modulating SALPR or Relaxin-3 activities by contacting a biological subject from a region that is suspected to be precancerous or cancerous with a modulator of the SALPR or Relaxin-3 protein, wherein said modulator partially or completely inhibits transcription of SALPR or Relaxin-3 gene, respectively.

Another aspect of the invention is to provide methods of making a pharmaceutical composition comprising: identifying a compound which is an inhibitor of SALPR or Relaxin-3 activity, including the oncogenic function or anti-apoptotic activity of SALPR or Relaxin-3; producing the compound; and optionally mixing the compound with suitable additives or other active agents.

Still another aspect of the invention is to provide a pharmaceutical composition obtainable by the methods described herein, wherein the composition comprises an antibody that blocks the oncogenic function or anti-apoptotic activity of SALPR or Relaxin-3.

Another aspect of the invention is to provide a pharmaceutical composition obtainable by the methods described herein, wherein the composition comprises an antibody that binds to a cell over-expressing SALPR or Relaxin-3 protein, thereby resulting in death or silencing of the cell.

Yet another aspect of the invention is to provide a pharmaceutical composition obtainable by the methods described herein, wherein the composition comprises a SALPR- or Relaxin-3-derived polypeptide or a fragment or a mutant thereof, wherein the polypeptide has inhibitory activity that blocks or inhibits the oncogenic function or anti-apoptotic activity of SALPR or Relaxin-3, respectively.

In still a further aspect, the invention provides methods for inducing an immune response in a mammal comprising contacting the mammal with SALPR or Relaxin-3 polypeptide or polynucleotide, or a fragment thereof, wherein the immune response produces antibodies and/or T cell immune response to protect the mammal from cancers, including a lung cancer, a colon cancer, an ovarian cancer, or a pancreatic cancer.

Another aspect of the invention is to provide methods of administering siRNA to a patient in need thereof, wherein the siRNA molecule is delivered in the form of a naked oligonucleotide, sense molecule, antisense molecule, and/or in a vector, wherein the siRNA interacts with SALPR or Relaxin-3 gene or their transcripts, wherein the vector is a plasmid, cosmid, bacteriophage, or a virus, wherein the virus is for example, a retrovirus, an adenovirus, or other suitable viral vector.

Another aspect of the invention is to provide methods of administering miRNA to a patient in need thereof, wherein the miRNA molecule is delivered in the form of a naked oligonucleotide, sense molecule, antisense molecule, and/or in a vector, wherein the miRNA interacts with SALPR or Relaxin-3 gene or their transcripts, wherein the vector is a plasmid, cosmid, bacteriophage, or a virus, wherein the virus is for example, a retrovirus, an adenovirus, or other suitable viral vector.

Still in another aspect, the invention provides methods of administering a decoy molecule to a patient in need thereof, wherein the molecule is delivered in the form of a naked oligonucleotide, sense molecule, antisense molecule, a decoy DNA molecule, and/or in a vector, wherein the molecule interacts with SALPR or Relaxin-3 gene, wherein the vector is a plasmid, cosmid, bacteriophage, or a virus, wherein the virus is for example, a retrovirus, an adenovirus, or other suitable viral vector.

In still a further aspect of the invention, SALPR or Relaxin-3 decoys, antisense, triple helix forming molecules, and ribozymes can be administered concurrently or consecutively in any proportion; for example, two of the above can be administered concurrently or consecutively in any proportion; or they can be administered singly (that is, decoys, triple helix forming molecules, antisense or ribozymes). Additionally, decoys, triple helix forming molecules, antisense and ribozymes having different sequences but directed against a given target (that is, SALPR or Relaxin-3) can be administered concurrently or consecutively in any proportion, including equimolar proportions. Thus, as is apparent to the skilled person in view of the teachings herein, one could choose to administer one SALPR or Relaxin-3 decoy molecule, triple helix forming molecules, antisense and/or ribozymes, and/or two different SALPR or Relaxin-3 decoys, triple helix forming molecules, antisense and/or ribozymes, and/or three different SALPR or Relaxin-3 decoys, triple helix forming molecules, antisense and/or ribozymes in any proportion, including equimolar proportions, for example. Of course, other permutations and proportions can be employed by the person skilled in the art.

Still in another aspect, the invention provides methods of administering SALPR- or Relaxin-3-siRNA and/or SALPR- or Relaxin-3-shRNA and/or SALPR- or Relaxin-3-miRNA to a patient in need thereof, wherein one or more of the above siRNA and/or shRNA and/or miRNA molecules are delivered in the form of a naked oligonucleotide, sense molecule, antisense molecule or a vector, wherein the siRNA(s) and/or shRNA(s) and/or miRNA(s) interact(s) with SALPR- or Relaxin-3 activity, wherein the vector is a plasmid, cosmid, bacteriophage or a virus, wherein the virus is, for example, a retrovirus, an adenovirus, a poxvirus, a herpes virus or other suitable viral vector. In other words, SALPR- or Relaxin-3-siRNAs and/or SALPR- or Relaxin-3-shRNAs and/or SALPR- or Relaxin-3-miRNAs can be administered concurrently or consecutively in any proportion; only two of the above can be administered concurrently or consecutively in any proportion; or they can be administered singly (that is, siRNAs or shRNAs or miRNAs targeting SALPR- or Relaxin-3). Additionally, siRNAs or shRNAs or miRNAs having different sequences but directed against a given target (that is, SALPR or Relaxin-3) can be administered concurrently or consecutively in any proportion, including equimolar proportions. Thus, as is apparent to the skilled person in view of the teachings herein, one could choose to administer one SALPR or Relaxin-3 siRNA or shRNA or miRNA and/or two different SALPR or Relaxin-3 siRNAs or shRNAs or miRNAs and/or three different SALPR or Relaxin-3 siRNAs or shRNAs or miRNAs in any proportion, including equimolar proportions, for example. Of course, other permutations and proportions can be employed by the person skilled in the art. Additionally, siRNAs or shRNAs or miRNAs can be employed together with one or more of decoys, triple helix forming molecules, antisense, ribozymes, and other functional molecules.

In another aspect, the present invention provides methods of blocking in vivo expression of a gene by administering a vector containing SALPR or Relaxin-3 siRNA or shRNA or miRNA, wherein the siRNA and/or shRNA and/or miRNA interacts with SALPR or Relaxin-3 activity, respectively, wherein the siRNA and/or shRNA and/or miRNA causes post-transcriptional silencing of SALPR or Relaxin-3 gene, respectively, or inhibits translation of RNA into protein, in a mammalian cell, for example, a human cell.

Yet, in another aspect, the present invention provides methods of treating cells ex vivo by administering a vector as described herein, wherein the vector is a plasmid, cosmid, bacteriophage, or a virus, such as a retrovirus or an adenovirus.

In its in vivo or ex vivo therapeutic applications, it is appropriate to administer siRNA and/or shRNA and/or miRNA using a viral or retroviral vector which enters the cell by transfection or infection. In particular, as a therapeutic product according to the invention, a vector can be a defective viral vector such as an adenovirus or a defective retroviral vector such as a murine retrovirus.

Another aspect of the invention provides methods of screening or validating potency of a molecule for SALPR or Relaxin-3 antagonist activity comprising, in any practical order, the steps of: contacting or exposing a cancer cell with the molecule; determining the level of SALPR or Relaxin-3 in the cell, thereby generating data for a test level; and comparing the test level to the level of SALPR or Relaxin-3, respectively, in the cell prior to contacting or exposing the molecule (initial or pre-exposed level), wherein a decrease in SALPR or Relaxin-3 in the test level indicates SALPR or Relaxin-3 antagonist activity of the molecule, wherein the level of SALPR or Relaxin-3 is determined by, for example, reverse transcription and polymerase chain reaction (RT-PCR), Northern hybridization, or microarray analysis.

In another aspect, the invention provides methods of screening or validating potency of a molecule for SALPR or Relaxin-3 antagonist activity comprising the steps of: contacting or exposing the molecule with SALPR or Relaxin-3 and determining the effect of the molecule on SALPR or Relaxin-3, respectively, wherein the effect can be determined via a binding assay or other appropriate assays, including RT-PCR, Northern hybridization, microarray analysis, two-hybrid assays such as GAL4 DNA binding domain based assays, EIA, blot assays, sandwich assays, and the like.

In another aspect, the invention provides methods of determining whether a molecule has SALPR or Relaxin-3 antagonist activity or validating potency of the molecule, wherein the method comprises, in any practical order, determining the level of SALPR or Relaxin-3 in a test sample containing cancer cells, thereby generating data for an initial level; contacting the molecule with the test sample to ultimately obtain a test level; and comparing the initial level to the test level, wherein no statistically significant decrease in SALPR or Relaxin-3 in the test level compared to the initial level indicates the molecule has no SALPR or Relaxin-3 antagonist activity, respectively; and eliminating the molecule from further evaluation or study.

In another aspect, the invention provides methods for selecting or validating potency of molecules having SALPR or Relaxin-3 antagonist activity, wherein the method comprises, in any practical order, determining the level of SALPR or Relaxin-3 in a test sample containing cancer cells, thereby generating data for an initial level; contacting the molecule with the test sample to ultimately obtain a test level; comparing the initial level to the test level, wherein no statistically significant decrease in SALPR or Relaxin-3 in the test level compared to the initial level indicates the molecule has no SALPR or Relaxin-3 antagonist activity, respectively; and eliminating the molecule from further evaluation or study.

Yet, in another aspect, the invention provides methods of screening or validating potency of a molecule for SALPR or Relaxin-3 antagonist activity comprising, in any practical order, the steps of: contacting a test sample containing cancer cells with the molecule; determining the level of SALPR or Relaxin-3 mRNA transcrips per cell, for example, by determining the overall level divided by the number of cells present in the sample, thereby generating data for a test level; and comparing the test level to the expression level of SALPR or Relaxin-3 mRNA transcrips per cell, for example, prior to contacting the molecule (initial level), wherein a decrease in expression of SALPR or Relaxin-3 in the test level indicates SALPR or Relaxin-3 antagonist activity of the molecule, respectively, wherein the expression level of SALPR or Relaxin-3 can be determined by, for example, binding assays or other appropriate assays, including RT-PCR, Northern hybridization, microarray analysis, two-hybrid assays such as GAL4 DNA binding domain based assays, EIA, blot assays, sandwich assays, and the like.

Still in another aspect, the invention provides methods of screening or validating potency of a molecule for SALPR or Relaxin-3 antagonist activity comprising, in any practical order, the steps of: determining the mRNA expression level of SALPR or Relaxin-3 in a test sample containing cancer cells, thereby generating data for an initial or a pre-test level expression of SALPR or Relaxin-3 mRNA; contacting the test sample with the molecule; determining the level of SALPR or Relaxin-3 mRNA transcrips per cell, for example, by determining the overall level divided by the number of cells present in the sample, thereby generating data for a test level; and comparing the test level to the initial or pre-test level expression of SALPR or Relaxin-3 mRNA transcrips per cell, for example, wherein a decrease in expression of SALPR or Relaxin-3 mRNA in the test level indicates SALPR or Relaxin-3 antagonist activity of the molecule, respectively. The expression level of SALPR or Relaxin-3 can be determined by, for example, binding assays or other appropriate assays, including RT-PCR, Northern hybridization, microarray analysis, two-hybrid assays such as GAL4 DNA binding domain based assays, blot assays, sandwich assays, and the like.

In another aspect, the invention provides methods for determining the level of SALPR or Relaxin-3 in a test sample for diagnosis of a cancer, for example, a lung cancer, a colon cancer, an ovarian cancer, or a pancreatic cancer, in a patient, comprising, in any practical order, obtaining a control sample; obtaining a test sample from the patient; contacting both the control and the test samples with anti-SALPR or anti-Relaxin-3 antibodies, determining the level of SALPR or Relaxin-3 in both the control and the test samples, by determining the overall level of SALPR or Relaxin-3 divided by the number of cells present in each sample; and comparing the level of SALPR or Relaxin-3, respectively, in the control and the test samples. A higher level of the SALPR or Relaxin-3 in the test sample obtained from the patient than that in the control sample indicates a cancer or a precancerous condition. The SALPR or Relaxin-3 level can be determined via binding assays or other appropriate assays, including RT-PCR, Northern hybridization, microarray analysis, two-hybrid assays such as GAL4 DNA binding domain based assays, EIA, blot assays, sandwich assays, and the like. Alternatively, a given level of SALPR or Relaxin-3, representative of the cancer-free population, that has been previously established based on measurements from normal, cancer-free animals, can be used as a control. A control data point from a reference database, based on data obtained from control samples representative of a cancer-free population, also can be used as a control.

In another aspect, the invention provides methods for determining the efficacy, such as potency, of a therapeutic treatment regimen in a patient, comprising, in any practical order, measuring at least one of SALPR or Relaxin-3 mRNA or SALPR or Relaxin-3 protein expression levels in a first sample obtained from the patient, thereby generating data for a pre-treatment level; administering the treatment regimen to the patient; measuring at least one of SALPR or Relaxin-3 mRNA or SALPR or Relaxin-3 protein expression levels in a second sample from the patient at a time following administration of the treatment regimen (test level); and comparing at least one of SALPR or Relaxin-3 mRNA or SALPR or Relaxin-3 protein expression levels in the first and the second samples, respectively, wherein data showing no statistically significant decrease in the levels in the second sample relative to the first sample indicates that the treatment regimen is not effective in the patient.

In another aspect, the invention provides methods for selecting test molecules having a therapeutic effect in a patient, comprising, in any practical order, measuring at least one of SALPR or Relaxin-3 mRNA or SALPR or Relaxin-3 protein expression levels in a first sample obtained from the patient, thereby generating data for a pre-treatment level; administering the test molecule to the patient; measuring at least one of SALPR or Relaxin-3 mRNA or SALPR or Relaxin-3 protein expression levels in a second sample from the patient at a time following administration of the test molecule, thereby generating a test level; comparing at least one of SALPR or Relaxin-3 mRNA or SALPR or Relaxin-3 protein expression levels in the first and the second samples, respectively, wherein data showing no statistically significant decrease in the levels in the second sample (test level) relative to the first sample (pre-treatment level) indicates that the test molecule is not effective in the patient; and eliminating the test molecule from further evaluation or study.

In another aspect, the invention provides methods for validating the potency of a therapeutic compound, wherein the method comprises, in any practical order, measuring SALPR or Relaxin-3 mRNA transcripts level in a first sample of cells, for example, lung cancer, colon cancer, ovarian cancer, or pancreatic cancer cells, wherein the cells may comprise an SALPR or Relaxin-3 amplicon, thereby generating data for a pre-treatment level; contacting the cells with the compound; measuring SALPR or Relaxin-3 mRNA transcripts level in a second sample from the cells at a time following contacting the compound, thereby generating data for a test level; and comparing the pre-treatment level to the test level, respectively, wherein a decrease in the test level relative to the pre-treatment level indicates that the compound is effective.

In another aspect, the invention provides methods for validating the potency of a therapeutic compound, wherein the method comprises, in any practical order, measuring SALPR or Relaxin-3 protein expression level in a first sample of cells, for example, lung cancer, colon cancer, ovarian cancer, or pancreatic cancer cells, wherein the cells may comprise an SALPR or Relaxin-3 amplicon, thereby generating data for a pre-treatment level; contacting the cells with the compound; measuring SALPR or Relaxin-3 protein expression level in a second sample from the cells at a time following contacting the compound, thereby generating data for a test level; and comparing the pre-treatment level to the test level, respectively, wherein a decrease in the test level relative to the pre-treatment level indicates that the compound is effective.

Yet in another aspect, the invention provides methods for validating the potency of a therapeutic compound, wherein the method comprises culturing a cell line comprising SALPR or Relaxin-3 amplicon in a suitable growth media; contacting the cell line with the compound; and examining the culture for cell death or suppression of cellular growth, wherein cellular death or suppression of growth indicates that the compound is effective.

Samples can be obtained from the same region or a different region of a subject. Typically, samples are taken in regions that are similar in terms of organ or tissue type and location in order to minimize variables.

The compounds, targets, assays, tests, inquiries and methodologies described herein can be employed in a variety of contexts, including diagnostic and therapeutic discovery, diagnostic and therapeutic development, safety and efficacy monitoring, compound and treatment regimen potency determination and validation, treatment assessment, comparative studies, marketing and the like. The information provided by the invention can be communicated to regulators, physicians and other healthcare providers, manufacturers, owners, investors, patients, and/or the general public. This information and the like can be used in exploratory research, pre-clinical and clinical settings, labeling, production, advertising, and sales, for example.

Unless otherwise defined, all technical and scientific terms used herein in their various grammatical forms have the same meaning as commonly understood by one of ordinary skill in the art to which this invention belongs. Although methods and materials similar to those described herein can be used in the practice or testing of the present invention, the preferred methods and materials are described below. In case of conflict, the present specification, including definitions, will control. In addition, the materials, methods, and examples are illustrative only and are not limiting.

Further features, objects, and advantages of the present invention are apparent in the claims and the detailed description that follows. It should be understood, however, that the detailed description and the specific examples, while indicating preferred aspects of the invention, are given by way of illustration only, since various changes and modifications within the spirit and scope of the invention will become apparent to those skilled in the art from this detailed description.

### BRIEF DESCRIPTION OF THE DRAWINGS

FIG. 1 depicts the epicenter mapping of human chromosome region 5p15.1-p14 amplicon, which includes SALPR locus. The number of DNA copies for each sample is plotted on the Y-axis, and the X-axis corresponds to nucleotide position based on Human Genome Project working draft sequence (http://genome.ucsc.edu/goldenPath/aug2001Tracks.html).

FIG. 2 shows epicenter of the genomic DNA locus containing SALPR gene in four lung tumor samples. Solid bar indicates Relaxin-3 gene in the amplified region.

FIG. 3 depicts Cluster analysis of DNA copy numbers of Relaxin-3, SALPR, G protein-coupled receptor 7 (LGR7), and GPCR142. Results are displayed in the format of Eisen dendrogram: gray shades indicate increase in DNA copy number, for example, tumors samples 263A1 and 4159A1 exhibit amplifications of both Relaxin-3 and SALPR.

FIG. 4 shows tumor growth (Mean±SEM) in athymic nude mice following implantation with about 5 million 3T3 transfectants. A total of 10 mice were used for each experimental (SALPR)/control (Vector) group and palpable/measurable tumors were recorded. Tumor growth was measured with a caliper in three perpendicular dimensions and recorded as mm³.

FIG. 5 shows tumor growth (Mean±SEM) in athymic nude mice following implantation with about 5 million 3T3 transfectants. A total of 6 mice for experimental (SALPR C terminal FLAG) and 5 mice for control (Vector only) group were used. Tumor growth was measured with a caliper in three perpendicular dimensions and recorded as mm³.

### DETAILED DESCRIPTION OF THE INVENTION

The present invention provides methods and compositions for the diagnosis, prevention, and treatment of tumors and cancers, for example, a lung cancer, a colon cancer, an ovarian cancer, or a pancreatic cancer, in mammals, for example, humans. The invention is based on the findings of novel attributes of the SALPR and Relaxin-3 genes. The SALPR and/or Relaxin-3 genes and their expressed protein products thus can be used diagnostically or as targets for therapy; and, they also can be used to identify compounds useful in the diagnosis, prevention, and therapy of tumors and cancers (for example, a lung cancer, a colon cancer, an ovarian cancer, or a pancreatic cancer).

The present invention also provides isolated amplified SALPR and Relaxin-3 genes. This invention also provides that the SALPR and Relaxin-3 genes are frequently amplified and/or overexpressed in tumor cells, for example, human lung tumor, colon tumor, ovarian tumor, or pancreatic tumor, and relates to methods and compositions associated with the diagnosis, prevention, monitoring, and treatment of cancers.

Homo sapiens Somatostatin- and Angiotensin-Like Peptide Receptor (SALPR):

SALPR, a putative seven-transmembrane domain receptor, a G-protein-coupled receptor (GPCR, also known as GPCR135), contains 469 amino acids and shares the highest amount of amino acid similarity with the somatostatin (35% with seven-transmembrane receptor SSTR5) and angiotensin (31% with angiotensin II receptor subtype ATI) receptors. SALPR and related mRNA are expressed in various organs in humans, including brain, particularly the substantia nigra and pituitary regions, and at low levels in the peripheral tissues (Matsumoto et al., Gene 248(1-2):183-189, 2000).

A full-length cDNA for SALPR has been cloned and the sequence has been submitted to GenBank database (Accession No. NM_{—}016568; SEQ ID NO:1). The SALPR DNA of 1857 nucleotides encodes a protein of 469 amino acids (GenBank Protein ID. NP_{—}057652.1; SEQ ID NO:2). The amino acid sequence encoded by the DNA for SALPR shows a high degree of identity to other SALPR family proteins. The human SALPR gene maps to chromosome 5p15.1-p14.

Several international applications and research articles (see International Publications WO 01/48189, EP 1 126 029, WO 00/24891, JP2000279183, WO 02/31111, WO 01/85791, and WO 02/61097; Matsumoto et al., Gene 248(1-2):183-189, 2000; O'Dowd et al., Gene 10;187(1):75-81, 1997; Kolakowski et al., FEBS Lett. 398(2-3):253-258, 1996; and Mukoyama et al., J Biol Chem 268(33):24539-24542, 1993) generally describe aspects of GPCR, somatostatin, and angiotensin related proteins, encoding genes and their expression products, however, amplification and overexpression of SALPR gene and its practical uses in cancer diagnosis and treatment have not been discussed.

Homo sapiens Relaxin-3:

Homo sapiens Relaxin-3 (H3) (RLN3) also is known as insulin-7 (INSL7). Relaxin-3 protein is known to bind and activate orphan leucine-rich repeat-containing G protein-coupled receptor 7 (LGR7). Human relaxin 3 (H3 relaxin) recently has been discovered as a novel ligand for relaxin receptors (Sudo et al. J Biol Chem. 278(10):7855-62, 2003). It was not known until recently that the relaxin-3 is an endogenous ligand of SALPR, the G-protein coupled receptor, GPCR135 (Liu et al. J Biol Chem. 278(50):50754-64, 2003) and a single orphan receptor, GPCR142 (Liu et al. J Biol Chem. 278(50):50765-70, 2003).

A full-length cDNA for Relaxin-3 has been cloned and the sequence has been submitted to GenBank database (Accession No. NM_{—}080864; SEQ ID NO:3). The Relaxin-3 DNA of 429 nucleotides encodes a protein of 142 amino acids (GenBank Protein ID. NP_{—}543140; SEQ ID NO:4). The human Relaxin-3 gene maps to chromosome 19p13.2.

Several investigators have generally described the role of relaxin-3 in neuropeptide signaling processes (Bathgate et al. J Biol Chem. 277(2):1148-57, 2002) and have speculated about its involvement in tumor progression (Ivell and Einspanier, Trends Endocrinol Metab. 13(8):343-8, 2002), however, amplification and overexpression of relaxin-3 gene and its practical uses in cancer diagnosis and treatment have not been discussed.

1. Definitions:

A “cancer” in an animal refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells.

The phrase “detecting a cancer” or “diagnosing or predicting a cancer or a cancer potential” refers to determining the presence or absence of cancer or a precancerous condition in an animal. “Detecting a cancer” also can refer to obtaining indirect evidence regarding the likelihood of the presence of precancerous or cancerous cells in the animal or assessing the predisposition of a patient to the development of a cancer. Detecting a cancer can be accomplished using the methods of this invention alone, in combination with other methods, or in light of other information regarding the state of health of the animal.

A “tumor,” as used herein, refers to all neoplastic cell growth and proliferation, whether malignant or benign, and all precancerous and cancerous cells and tissues.

The term “precancerous” refers to cells or tissues having characteristics relating to changes that may lead to malignancy or cancer. Examples include adenomatous growths in lung, colon, ovary, or pancreas, tissues, or conditions, for example, dysplastic nevus syndrome, a precursor to malignant melanoma of the skin. Examples also include, abnormal neoplastic, in addition to dysplastic nevus syndromes, polyposis syndromes, prostatic dysplasia, and other such neoplasms, whether the precancerous lesions are clinically identifiable or not.

A “differentially expressed gene transcript”, as used herein, refers to a gene, including an oncogene, transcript that is found in different numbers of copies in different cell or tissue types of an organism having a tumor or cancer, for example, a lung cancer, a colon cancer, an ovarian cancer, or a pancreatic cancer, compared to the numbers of copies or state of the gene transcript found in the cells of the same tissue in a healthy organism, or in the cells of the same tissue in the same organism. Multiple copies of gene transcripts may be found in an organism having the tumor or cancer, while fewer copies of the same gene transcript are found in a healthy organism or healthy cells of the same tissue in the same organism, or vice-versa.

A “differentially expressed gene,” can be a target, fingerprint, or pathway gene. For example, a “fingerprint gene”, as used herein, refers to a differentially expressed gene whose expression pattern can be used as a prognostic or diagnostic marker for the evaluation of tumors and cancers, or which can be used to identify compounds useful for the treatment of tumors and cancers, for example, lung cancer, colon cancer, ovarian cancer, or pancreatic cancer. For example, the effect of a compound on the fingerprint gene expression pattern normally displayed in connection with tumors and cancers can be used to evaluate the efficacy, such as potency, of the compound as a tumor and cancer treatment, or can be used to monitor patients undergoing clinical evaluation for the treatment of tumors and cancer.

A “fingerprint pattern”, as used herein, refers to a pattern generated when the expression pattern of a series (which can range from two up to all the fingerprint genes that exist for a given state) of fingerprint genes is determined. A fingerprint pattern also may be referred to as an “expression profile”. A fingerprint pattern or expression profile can be used in the same diagnostic, prognostic, and compound identification methods as the expression of a single fingerprint gene.

A “target gene”, as used herein, refers to a differentially expressed gene in which modulation of the level of gene expression or of gene product activity prevents and/or ameliorates tumor and cancer, for example, lung cancer, colon cancer, ovarian cancer, or pancreatic cancer, symptoms. Thus, compounds that modulate the expression of a target gene, the target gene, or the activity of a target gene product can be used in the diagnosis, treatment or prevention of tumors and cancers. A particular target gene of the present invention is the SALPR or Relaxin-3 gene.

In general, a “gene” is a region on the genome that is capable of being transcribed to an RNA that either has a regulatory function, a catalytic function, and/or encodes a protein. An eukaryotic gene typically has introns and exons, which may organize to produce different RNA splice variants that encode alternative versions of a mature protein. The skilled artisan will appreciate that the present invention encompasses all SALPR- and Relaxin-3-encoding transcripts that may be found, including splice variants, allelic variants and transcripts that occur because of alternative promoter sites or alternative poly-adenylation sites. A “full-length” gene or RNA therefore encompasses any naturally occurring splice variants, allelic variants, other alternative transcripts, splice variants generated by recombinant technologies which bear the same function as the naturally occurring variants, and the resulting RNA molecules. A “fragment” of a gene, including an oncogene, can be any portion from the gene, which may or may not represent a functional domain, for example, a catalytic domain, a DNA binding domain, etc. A fragment may preferably include nucleotide sequences that encode for at least 25 contiguous amino acids, and preferably at least about 30, 40, 50, 60, 65, 70, 75 or more contiguous amino acids or any integer thereabout or therebetween.

“Pathway genes”, as used herein, are genes that encode proteins or polypeptides that interact with other gene products involved in tumors and cancers. Pathway genes also can exhibit target gene and/or fingerprint gene characteristics.

A “detectable” RNA expression level, as used herein, means a level that is detectable by standard techniques currently known in the art or those that become standard at some future time, and include for example, differential display, RT (reverse transcriptase)-coupled polymerase chain reaction (PCR), Northern Blot, and/or RNase protection analyses. The degree of differences in expression levels need only be large enough to be visualized or measured via standard characterization techniques.

As used herein, the term “transformed cell” means a cell into which (or into predecessor or an ancestor of which) a nucleic acid molecule encoding a polypeptide of the invention has been introduced, by means of, for example, recombinant DNA techniques or viruses.

The nucleic acid molecules of the invention, for example, the SALPR and Relaxin-3 genes or their subsequences, can be inserted into a vector, as described below, which will facilitate expression of the insert. The nucleic acid molecules and the polypeptides they encode can be used directly as diagnostic or therapeutic agents, or can be used (directly in the case of the polypeptide or indirectly in the case of a nucleic acid molecule) to generate antibodies that, in turn, are clinically useful as a therapeutic or diagnostic agent. Accordingly, vectors containing the nucleic acids of the invention, cells transfected with these vectors, the polypeptides expressed, and antibodies generated against either the entire polypeptide or an antigenic fragment thereof, are among the aspects of the invention.

A “structural gene” is a DNA sequence that is transcribed into messenger RNA (mRNA) which is then translated into a sequence of amino acids characteristic of a specific polypeptide.

An “isolated DNA molecule” is a fragment of DNA that has been separated from the chromosomal or genomic DNA of an organism. Isolation also is defined to connote a degree of separation from original source or surroundings. For example, a cloned DNA molecule encoding an avidin gene is an isolated DNA molecule. Another example of an isolated DNA molecule is a chemically-synthesized DNA molecule, or enzymatically-produced cDNA, that is not integrated in the genomic DNA of an organism. Isolated DNA molecules can be subjected to procedures known in the art to remove contaminants such that the DNA molecule is considered purified, that is, towards a more homogeneous state.

“Complementary DNA” (cDNA), often referred to as “copy DNA”, is a single-stranded DNA molecule that is formed from an mRNA template by the enzyme reverse transcriptase. Typically, a primer complementary to portions of the mRNA is employed for the initiation of reverse transcription. Those skilled in the art also use the term “cDNA” to refer to a double-stranded DNA molecule that comprises such a single-stranded DNA molecule and its complement DNA strand.

The term “expression” refers to the biosynthesis of a gene product. For example, in the case of a structural gene, expression involves transcription of the structural gene into mRNA and the translation of mRNA into one or more polypeptides.

The term “amplification” refers to amplification, duplication, multiplication, or multiple expression of nucleic acids or a gene, in vivo or in vitro, yielding about 3.0 fold or more copies. For example, amplification of the SALPR or Relaxin-3 gene resulting in a copy number greater than or equal to 3.0 is deemed to have been amplified. However, an increase in SALPR or Relaxin-3 gene copy number less than 3.0 fold can still be considered as an amplification of the gene. The 3.0 fold figure is due to current detection limit, rather than a biological state.

The term “amplicon” refers to an amplification product containing one or more genes, which can be isolated from a precancerous or a cancerous cell or a tissue. SALPR or Relaxin-3 amplicon is a result of amplification, duplication, multiplication, or multiple expression of nucleic acids or a gene, in vivo or in vitro. “Amplicon”, as defined herein, also includes a completely or partially amplified SALPR and/or Relaxin-3 genes. For example, an amplicon comprising a polynucleotide having at least about 90% sequence identity to SEQ ID NO:1 (SALPR), SEQ ID NO:3 (Relaxin-3), or a fragment thereof.

A “cloning vector” is a nucleic acid molecule, for example, a plasmid, cosmid, or bacteriophage that has the capability of replicating autonomously in a host cell. Cloning vectors typically contain (i) one or a small number of restriction endonuclease recognition sites at which foreign DNA sequences can be inserted in a determinable fashion without loss of an essential biological function of the vector, and (ii) a marker gene that is suitable for use in the identification and selection of cells transformed or transfected with the cloning vector. Marker genes include genes that provide tetracycline resistance or ampicillin resistance, for example.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

A “recombinant host” may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell.

“Antisense RNA”: In eukaryotes, RNA polymerase catalyzes the transcription of a structural gene to produce mRNA. A DNA molecule can be designed to contain an RNA polymerase template in which the RNA transcript has a sequence that is complementary to that of a preferred mRNA. The RNA transcript is termed an “antisense RNA”. Antisense RNA molecules can inhibit mRNA expression (for example, Rylova et al., Cancer Res, 62(3):801-8, 2002; Shim et al., Int. J. Cancer, 94(1):6-15, 2001).

“Antisense DNA” or “DNA decoy” or “decoy molecule”: With respect to a first nucleic acid molecule, a second DNA molecule or a second chimeric nucleic acid molecule that is created with a sequence which is a complementary sequence or homologous to the complementary sequence of the first molecule or portions thereof, is referred to as the “antisense DNA” or “DNA decoy” or “decoy molecule” of the first molecule. The term “decoy molecule” also includes a nucleic acid molecule, which may be single or double stranded, that comprises DNA or PNA (peptide nucleic acid) (Mischiati et al., Int. J. Mol. Med., 9(6):633-9, 2002), and that contains a sequence of a protein binding site, preferably a binding site for a regulatory protein and more preferably a binding site for a transcription factor. Applications of antisense nucleic acid molecules, including antisense DNA and decoy DNA molecules are known in the art, for example, Morishita et al., Ann. N Y Acad. Sci., 947:294-301, 2001; Andratschke et al., Anticancer Res, 21:(5)3541-3550, 2001. Antisense DNA or PNA molecules can inhibit, block, or regulate function and/or expression of a SALPR or a Relaxin-3 gene. Antisense and decoys can have different sequences, but can be directed against a SALPR or a Relaxin-3 and can be administered concurrently or consecutively in any proportion, including equimolar proportions.

The term “operably linked” is used to describe the connection between regulatory elements and a gene or its coding region. That is, gene expression is typically placed under the control of certain regulatory elements, including constitutive or inducible promoters, tissue-specific regulatory elements, and enhancers. Such a gene or coding region is said to be “operably linked to” or “operatively linked to” or “operably associated with” the regulatory elements, meaning that the gene or coding region is controlled or influenced by the regulatory element.

“Sequence homology” is used to describe the sequence relationships between two or more nucleic acids, polynucleotides, proteins, or polypeptides, and is understood in the context of and in conjunction with the terms including: (a) reference sequence, (b) comparison window, (c) sequence identity, (d) percentage of sequence identity, and (e) substantial identity or “homologous.”

(a) A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

(b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions, or deletions (i.e., gaps) compared to the reference sequence (which does not comprise additions, substitutions, or deletions) for optimal alignment of the two sequences. Generally, the comparison window is at least 20 contiguous nucleotides in length, and optionally can be 30, 40, 50, 100, or longer. Those of skill in the art understand that to avoid a misleadingly high similarity to a reference sequence due to inclusion of gaps in the polynucleotide sequence a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73: 237-244, 1988; Corpet, et al., Nucleic Acids Research, 16:881-90, 1988; Huang, et al., Computer Applications in the Biosciences, 8:1-6, 1992; and Pearson, et al., Methods in Molecular Biology, 24:7-331, 1994. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer to the value obtained using the BLAST 2.0 suite of programs, or their successors, using default parameters. Altschul et al., Nucleic Acids Res, 2:3389-3402, 1997. It is to be understood that default settings of these parameters can be readily changed as needed in the future.

As those ordinary skilled in the art will understand, BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163, 1993) and XNU (Claverie and States, Comput. Chem., 17:191-1, 1993) low-complexity filters can be employed alone or in combination.

(c) “Sequence identity” or “identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule. Where sequences differ in conservative substitutions, the percent sequence identity may be adjusted upwards to correct for the conservative nature of the substitution. Sequences which differ by such conservative substitutions are said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

(d) “Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) (i) The tern “substantial identity” or “homologous” in their various grammatical forms in the context of polynucleotides means that a polynucleotide comprises a sequence that has a desired identity, for example, at least 60% identity, preferably at least 70% sequence identity, more preferably at least 80%, still more preferably at least 90% and even more preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 90%, and even more preferably at least 95%.

Another indication that nucleotide sequences are substantially identical if two molecules hybridize to each other under stringent conditions. However, nucleic acids which do not hybridize to each other under stringent conditions are still substantially identical if the polypeptides which they encode are substantially identical. This may occur, for example, when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid, although such cross-reactivity is not required for two polypeptides to be deemed substantially identical.

(e) (ii) The term “substantial identity” or “homologous” in their various grammatical forms in the context of peptides indicates that a peptide comprises a sequence that has a desired identity, for example, at least 60% identity, preferably at least 70% sequence identity to a reference sequence, more preferably 80%, still more preferably 85%, even more preferably at least 90% or 95% sequence identity to the reference sequence over a specified comparison window. Preferably, optimal alignment is conducted using the homology alignment algorithm of Needleman and Wunsch, J. Mol. Biol., 48:443, 1970. An indication that two peptide sequences are substantially identical is that one peptide is immunologically reactive with antibodies raised against the second peptide, although such cross-reactivity is not required for two polypeptides to be deemed substantially identical. Thus, a peptide is substantially identical to a second peptide, for example, where the two peptides differ only by a conservative substitution. Peptides which are “substantially similar” share sequences as noted above except that residue positions which are not identical may differ by conservative amino acid changes. Conservative substitutions typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine, and others as known to the skilled person.

“Biological subject” as used herein refers to a target biological object obtained, reached, or collected in vivo, ex-vivo, or in situ, that contains or is suspected of containing nucleic acids or polypeptides of SALPR or Relaxin-3. A biological subject is typically of eukaryotic nature, for example, insects, protozoa, birds, fish, reptiles, and preferably a mammal, for example, rat, mouse, cow, dog, guinea pig, or rabbit, and more preferably a primate, for example, chimpanzees, or humans such as a patient in need of diagnostic review, treatment and/or monitoring of therapy.

“Biological sample” as used herein refers to a sample obtained from a biological subject, including sample of biological tissue or fluid origin, obtained, reached, or collected in vivo, ex-vivo, or in situ, that contains or is suspected of containing nucleic acids or polypeptides of SALPR or Relaxin-3. A biological sample also includes samples from a region of a biological subject containing precancerous or cancer cells or tissues. Such samples can be, but are not limited to, organs, tissues, fractions and cells isolated from mammals including, humans such as a patient, mice, and rats. Biological samples also may include sections of the biological sample including tissues, for example, frozen sections taken for histologic purposes. A biological sample is typically of an eukaryotic origin, for example, insects, protozoa, birds, fish, reptiles, and preferably a mammal, for example, rat, mouse, cow, dog, guinea pig, or rabbit, and more preferably a primate, for example, chimpanzees or humans. A biological sample, as described herein, can be: a “control” or a “control sample” or a “test sample”.

A “control ” refers to a representative of healthy, cancer-free biological subject or information obtained from a different individual or a normalized value, which can be based on baseline data obtained from a population or other acceptable sources. A control also can refer to a given level of SALPR or Relaxin-3, representative of the cancer-free population, that has been previously established based on measurements from normal, cancer-free animals. A control also can be a reference data point in a database based on data obtained from control samples representative of a cancer-free population. Further, a control can be established by a specific age, sex, ethnicity or other demographic parameters. In some situations, the control is implicit in the particular measurement. A typical control level for a gene is two copies per cell. An example of an implicit control is where a detection method can only detect SALPR or Relaxin-3, or the corresponding gene copy number, when a level higher than that typical of a normal, cancer-free animal is present. Another example is in the context of an immunohistochemical assay where the control level for the assay is known. Other instances of such controls are within the knowledge of the skilled person.

A “control sample” refers to a sample of biological material representative of healthy, cancer-free animals or a normal biological subject obtained from a cancer-free population. The level of SALPR or Relaxin-3 in a control sample, or the encoding corresponding gene copy number, is desirably typical of the general population of normal, cancer-free animals of the same species. This sample either can be collected from an animal for the purpose of being used in the methods described in the present invention or it can be any biological material representative of normal, cancer-free animals suitable for use in the methods of this invention. A control sample also can be obtained from normal tissue from the animal that has cancer or is suspected of having cancer.

A “test sample” as used herein refers to a biological sample, including sample of biological tissue or fluid origin, obtained, reached, or collected in vivo, ex-vivo, or in situ, that contains or is suspected of containing nucleic acids or polypeptides of SALPR or Relaxin-3. A test sample also includes biological samples containing precancerous or cancer cells or tissues. Such test samples can be, but are not limited to, organs, tissues, fractions and cells isolated from mammals including, humans such as a patient, mice, and rats. A test sample also may include sections of the biological sample including tissues, for example, frozen sections taken for histologic purposes.

“Providing a biological subject, a biological sample, or a test sample” means to obtain a biological subject in vivo, ex-vivo, or in situ, including tissue or cell sample for use in the methods described in the present invention. Most often, this will be done by removing a sample of cells from an animal, but also can be accomplished in vivo, ex-vivo, or in situ, or by using previously isolated cells (for example, isolated from another person, at another time, and/or for another purpose).

“Data” includes, but is not limited to, information obtained that relates to “biological sample”, “test sample”, “control sample”, and/or “control”, as described above, wherein the information is applied in generating a test level for diagnostics, prevention, monitoring or therapeutic use. The present invention relates to methods for comparing and compiling data wherein the data is stored in electronic or paper formats. Electronic format can be selected from the group consisting of electronic mail, disk, compact disk (CD), digital versatile disk (DVD), memory card, memory chip, ROM or RAM, magnetic optical disk, tape, video, video clip, microfilm, internet, shared network, shared server and the like; wherein data is displayed, transmitted or analyzed via electronic transmission, video display, telecommunication, or by using any of the above stored formats; wherein data is compared and compiled at the site of sampling specimens or at a location where the data is transported following a process as described above.

“Overexpression” of a SALPR or a Relaxin-3 gene or an “increased,” or “elevated,” level of a SALPR or a Relaxin-3 ribonucleotide or protein refers to a level of SALPR or Relaxin-3 ribonucleotide or polypeptide that, in comparison with a control level of SALPR or Relaxin-3, is detectably higher. Comparison may be carried out by statistical analyses on numeric measurements of the expression; or, it may be done through visual examination of experimental results by qualified researchers.

A level of SALPR or Relaxin-3 ribonucleotide or polypeptide, that is “expected” in a control sample refers to a level that represents a typical, cancer-free sample, and from which an elevated, or diagnostic, presence of SALPR or Relaxin-3 polypeptide or polynucleotide, can be distinguished. Preferably, an “expected” level will be controlled for such factors as the age, sex, medical history, etc. of the mammal, as well as for the particular biological subject being tested.

The phrase “functional effects” in the context of an assay or assays for testing compounds that modulate SALPR or Relaxin-3 activity includes the determination of any parameter that is indirectly or directly under the influence of SALPR or Relaxin-3, for example, a functional, physical, or chemical effect, for example, SALPR or Relaxin-3 activity, the ability to induce gene amplification or overexpression in cancer cells, and to aggravate cancer cell proliferation. “Functional effects” include in vitro, in vivo, and ex vivo activities.

“Determining the functional effect” refers to assaying for a compound that increases or decreases a parameter that is indirectly or directly under the influence of SALPR or Relaxin-3, for example, functional, physical, and chemical effects. Such functional effects can be measured by any means known to those skilled in the art, for example, changes in spectroscopic characteristics (for example, fluorescence, absorbance, refractive index), hydrodynamic (for example, shape), chromatographic, or solubility properties for the protein, measuring inducible markers or transcriptional activation of SALPR or Relaxin-3; measuring binding activity or binding assays, for example, substrate binding, and measuring cellular proliferation; measuring signal transduction; or measuring cellular transformation; or other appropriate assay, including reverse transcription and polymerase chain reaction (RT-PCR), Northern hybridization, microarray analysis, enzyme immuno assay (EIA), two-hybrid assays such as GAL4 DNA binding domain based assays, blot assays, sandwich assays, and the like.

“Inhibitors,” “activators,” “modulators,” and “regulators” refer to molecules that activate, inhibit, modulate, regulate and/or block an identified function. Any molecule having potential to activate, inhibit, modulate, regulate and/or block an identified function can be a “test molecule” or a “production molecule” or an “in-process molecule”, as described herein. A “test molecule” refers to uncharacterized or partially characterized molecules, natural or artifical, that may have the potential of anti-apoptotic activity of SALPR or Relaxin-3 and under investigation for potential to activate, inhibit, modulate, regulate and/or block an identified function. A “production molecule” or an “in-process molecule” refers to molecules that are characterized and/or identified as having the ability to activate, inhibit, modulate, regulate and/or block an identified function of SALPR or Relaxin-3. A “production molecule” or an “in-process molecule” can be validated for potency to activate, inhibit, modulate, regulate and/or block an identified function. For example, referring to oncogenic function or anti-apoptotic activity of SALPR or Relaxin-3, such molecules may be identified using in vitro and in vivo assays of SALPR or Relaxin-3, respectively. Inhibitors are compounds that partially or totally block SALPR or Relaxin-3, respectively, decrease, prevent, or delay their activation, or desensitize their cellular response. This may be accomplished by binding to SALPR or Relaxin-3 proteins directly or via other intermediate molecules. An antagonist or an antibody that blocks SALPR or Relaxin-3 activity, including inhibition of oncogenic function or anti-apoptotic activity of SALPR or Relaxin-3, respectively, is considered to be such an inhibitor.

One type of inhibitor is the soluble receptor trap. Soluble receptors provide effective traps for their ligands, which bind the ligands with affinities in the picomolar range, often without creating problematic intermediates. A soluble receptor trap for SALPR or Relaxin-3 proteins can act as an antagonist. The soluble receptor ligand trap functions as an antagonist by sequestering SALPR or Relaxin-3 and thus rendering unavailable to interact with the native receptors on SALPR- or Relaxin-3-responsive cells, respectively.

An effective antagonist of SALPR or Relaxin-3, such as a soluble receptor trap, can comprise heterodimers of the extracellular domains of SALPR or Relaxin-3 receptor, respectively, thus rendering SALPR or Relaxin-3 unavailable to interact with the native receptors on SALPR- or Relaxin-3-responsive cells, respectively.

Soluble ligand binding domains from extracellular portion of receptors have proven to be effective as traps for ligands (Bargetzi, et al., Cancer Res., 53:4010-4013, 1993; Mohler, et al., J. Immunol., 151:1548-1561,1993; Narazaki, et al., Blood, 82:1120-1126, 1993).

The heterodimeric receptors can be engineered using fusion regions, as described in published WO93/10151, published May 27, 1993, which describes production of beta receptor heterodimers, or they can be prepared by crosslinking of extracellular domains by chemical methodologies.

Technology known in the art also allows the engineering of different heteromeric soluble receptor ligand traps, which by virtue of their design may have additional beneficial characteristics such as stability, Fc-receptor-mediated clearance, or reduced effector functions (such as complement fixation). Furthermore, the technology described will be suitable for the engineering of any heteromeric protein in mammalian or other suitable protein expression systems, including but not limited to heteromeric molecules which employ receptors, ligands, and catalytic components such as enzymes or catalytic antibodies.

Activators are compounds that bind to SALPR or Relaxin-3 protein directly or via other intermediate molecules, thereby increasing or enhancing their activity, stimulating or accelerating their activation, or sensitizing their cellular response. An agonist of SALPR or Relaxin-3 is considered to be such an activator. A modulator can be an inhibitor or activator. A modulator may or may not bind SALPR or Relaxin-3 or their protein directly; it affects or changes the activity or activation of SALPR or Relaxin-3 or the cellular sensitivity to SALPR or Relaxin-3, respectively. A modulator also may be a compound, for example, a small molecule, that inhibits transcription of SALPR or Relaxin-3 mRNA. A regulator of SALPR or Relaxin-3 gene includes any element, for example, nucleic acid, peptide, polypeptide, protein, peptide nucleic acid or the like, that influences and/or controls the transcription/expression of SALPR or Relaxin-3 gene, respectively, or their coding region.

The group of inhibitors, activators, modulators and regulators of this invention also includes genetically modified versions of SALPR or Relaxin-3, for example, versions with altered activity. Thus, unless otherwise indicated, the group is inclusive of the naturally occurring protein as well as synthetic ligands, antagonists, agonists, antibodies, small chemical molecules and the like.

“Assays for inhibitors, activators, modulators, or regulators” refer to experimental procedures including, for example, expressing SALPR or Relaxin-3 in vitro, in cells, applying putative inhibitor, activator, modulator, or regulator compounds, and then determining the functional effects on SALPR or Relaxin-3 activity or transcription, as described above. Samples that contain or are suspected of containing SALPR or Relaxin-3 are treated with a potential activator, inhibitor, or modulator. The extent of activation, inhibition, or change is examined by comparing the activity measurement from the samples of interest to control samples. A threshold level is established to assess activation or inhibition. For example, inhibition of a SALPR or Relaxin-3 polypeptides are considered achieved when the SALPR or Relaxin-3 activity value relative to the control is 80% or lower. Similarly, activation of a SALPR or a Relaxin-3 polypeptides are considered achieved when the SALPR or Relaxin-3 activity value relative to the control is two or more fold higher.

The terms “isolated,” “purified,” and “biologically pure” each refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. Various levels of purity may be applied as needed according to this invention in the different methodologies set forth herein; the customary purity standards known in the art may be used if no standard is otherwise specified.

An “isolated nucleic acid molecule” can refer to a nucleic acid molecule, depending upon the circumstance, that is separated from the 5′ and 3′ coding sequences of genes or gene fragments contiguous in the naturally occurring genome of an organism. The term “isolated nucleic acid molecule” also includes nucleic acid molecules which are not naturally occurring, for example, nucleic acid molecules created by recombinant DNA techniques.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., Nucleic Acid Res, 19:081, 1991; Ohtsuka et al., J. Biol. Chem., 260:2600-2608, 1985; Rossolini et al., Mol. Cell Probes, 8:91-98, 1994). The term nucleic acid can be used interchangeably with gene, cDNA, mRNA, oligonucleotide, and polynucleotide.

A “host cell” is a naturally occurring cell or a transformed cell or a transfected cell that contains an expression vector and supports the replication or expression of the expression vector. Host cells may be cultured cells, explants, cells in vivo, and the like. Host cells may be prokaryotic cells, for example, E. coli, or eukaryotic cells, for example, yeast, insect, amphibian, or mammalian cells, for example, Vero, CHO, HeLa, and others.

A “cell line” refers to cultured cells that are immortal and can undergone passaging. Passaging refers to moving cultured cells from one culture chamber to another so that the cultured cells can be propagated to the subsequent generation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, γ-carboxyglutamate, and O-phosphoserine, phosphothreonine. “Amino acid analogs” refer to compounds that have the same basic chemical structure as a naturally occurring amino acid, i.e., a carbon that is bound to a hydrogen, a carboxyl group, an amino group, and an R group, for example, homoserine, norleucine, methionine sulfoxide, methionine methyl sulfonium. Such analogs have modified R groups (for example, norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid. Amino acids and analogs are well known in the art.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” apply to both amino acid and nucleic acid sequences. With respect to particular nucleic acid sequences, conservatively modified variants refers to those nucleic acids which encode identical or similar amino acid sequences and include degenerate sequences. For example, the codons GCA, GCC, GCG and GCU all encode alanine. Thus, at every amino acid position where an alanine is specified, any of these codons can be used interchangeably in constructing a corresponding nucleotide sequence. The resulting nucleic acid variants are conservatively modified variants, since they encode the same protein (assuming that is the only alternation in the sequence). One skilled in the art recognizes that each codon in a nucleic acid, except for AUG (sole codon for methionine) and UGG (tryptophan), can be modified conservatively to yield a functionally-identical peptide or protein molecule.

As to amino acid sequences, one skilled in the art will recognize that substitutions, deletions, or additions to a polypeptide or protein sequence which alter, add or delete a single amino acid or a small number (typically less than about ten) of amino acids is a “conservatively modified variant” where the alteration results in the substitution of an amino acid with a chemically similar amino acid. Conservative substitutions are well known in the art and include, for example, the changes of: alanine to serine; arginine to lysine; asparigine to glutamine or histidine; aspartate to glutamate; cysteine to serine; glutamine to asparigine; glutamate to aspartate; glycine to proline; histidine to asparigine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine. Other conservative and semi-conservative substitutions are known in the art and can be employed in practice of the present invention.

The terms “protein”, “peptide” and “Polypeptide” each are used herein to describe any chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length, naturally occurring proteins as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells.

The polypeptides of the invention can be chemically synthesized or synthesized by recombinant DNA methods; or, they can be purified from tissues in which they are naturally expressed, according to standard biochemical methods of purification.

Also included in the invention are “functional polypeptides,” which possess one or more of the biological functions or activities of a protein or polypeptide of the invention. These functions or activities include the ability to bind some or all of the proteins which normally bind to SALPR or Relaxin-3 protein.

The functional polypeptides may contain a primary amino acid sequence that has been modified from that considered to be the standard sequence of SALPR or Relaxin-3 protein described herein. Preferably these modifications are conservative amino acid substitutions, as described herein.

A “label” or a “detectable moiety” is a composition that when linked with the nucleic acid or protein molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. A “labeled nucleic acid or oligonucleotide probe” is one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe may be detected by detecting the presence of the label bound to the nucleic acid or probe.

As used herein a “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not unduly interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

The phrase “selectively (or specifically) hybridizes to” refers to the binding, duplexing, or hybridizing of a molecule only to a particular nucleotide sequence under stringent hybridization conditions when that sequence is present in a complex mixture (for example, total cellular or library DNA or RNA).

The phrase “stringent hybridization conditions” refers to conditions under which a probe will hybridize to its target complementary sequence, typically in a complex mixture of nucleic acids, but to no other sequences. Stringent conditions are sequence-dependent and circumstance-dependent; for example, longer sequences can hybridize with specificity at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). In the context of the present invention, as used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other.

Generally, stringent conditions are selected to be about 5 to 10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions also may be achieved with the addition of destabilizing agents, for example, formamide. For selective or specific hybridization, a positive signal is at least two times background, preferably 10 times background hybridization.

Exemplary stringent hybridization conditions can be as following, for example: 50% formamide, 5×SSC and 1% SDS, incubating at 42° C., or 5×SSC and 1% SDS, incubating at 65° C., with wash in 0.2×SSC and 0.1% SDS at about 65° C. Alternative conditions include, for example, conditions at least as stringent as hybridization at 68° C. for 20 hours, followed by washing in 2×SSC, 0.1% SDS, twice for 30 minutes at about 55° C. and three times for 15 minutes at about 60° C. Another alternative set of conditions is hybridization in 6×SSC at about 45° C., followed by one or more washes in 0.2×SSC, 0.1% SDS at about 50-65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C. to 95° C. for 30 sec. to 2 min., an annealing phase lasting 30 sec. to 2 min., and an extension phase of about 72° C. for 1 to 2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The terms “about” or “approximately” in the context of numerical values and ranges refers to values or ranges that approximate or are close to the recited values or ranges such that the invention can perform as intended, such as having a desired amount of nucleic acids or polypeptides in a reaction mixture, as is apparent to the skilled person from the teachings contained herein. This is due, at least in part, to the varying properties of nucleic acid compositions, age, race, gender, anatomical and physiological variations and the inexactitude of biological systems. Thus, these terms encompass values beyond those resulting from systematic error.

“Antibody” refers to a polypeptide comprising a framework region encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 2 kDa) and one “heavy” chain (up to about 70 kDa). Antibodies exist, for example, as intact immunoglobulins or as a number of well-characterized fragments produced by digestion with various peptidases. While various antibody fragments are defined in terms of the digestion of an intact antibody, one of skill in the art will appreciate that such fragments may be synthesized de novo chemically or via recombinant DNA methodologies. Thus, the term antibody, as used herein, also includes antibody fragments produced by the modification of whole antibodies, those synthesized de novo using recombinant DNA methodologies (for example, single chain Fv), humanized antibodies, and those identified using phage display libraries (see, for example, Knappik et al., J. Mol. Biol., 296:57-86, 2000; McCafferty et al., Nature, 348:2-4, 1990), for example. For preparation of antibodies—recombinant, monoclonal, or polyclonal antibodies—any technique known in the art can be used with this invention (see, for example, Kohler & Milstein, Nature, 256(5517):495-497, 1975; Kozbor et al., Immunology Today, 4:72, 1983; Cole et al., pp. 77-96 in Monoclonal Antibodies and Cancer Therapy, Alan R. Liss, Inc., 1998).

Techniques for the production of single chain antibodies (See U.S. Pat. No. 4,946,778) can be adapted to produce antibodies to polypeptides of this invention. Transgenic mice, or other organisms, for example, other mammals, may be used to express humanized antibodies. Phage display technology also can be used to identify antibodies and heteromeric Fab fragments that specifically bind to selected antigens (see, for example, McCafferty et al., Nature, 348:2-4, 1990; Marks et al., Biotechnology, 10(7) :779-783, 1992).

The term antibody is used in the broadest sense including agonist, antagonist, and blocking or neutralizing antibodies.

“Blocking antibody” is a type of antibody, as described above, that refers to a polypeptide comprising variable and framework regions encoded by an immunoglobulin gene or fragments, homologues, analogs or mimetics thereof that specifically binds and blocks biological activities of an antigen; for example, a blocking antibody to SALPR or Relaxin-3 blocks the oncogenic function or anti-apoptotic activity of SALPR or Relaxin-3 gene, respectively. A blocking antibody binds to critical regions of a polypeptide and thereby inhibits its function. Critical regions include protein-protein interaction sites, such as active sites, functional domains, ligand binding sites, and recognition sites. Blocking antibodies may be induced in mammals, for example in human, by repeated small injections of antigen, too small to produce strong hypersensitivity reactions. See Bellanti J A, Immunology, WB Saunders Co., p.131-368 (1971). Blocking antibodies can play an important role in blocking the function of a marker protein and inhibiting tumorigenic growth. See, for example, Jopling et al., J. Biol. Chem., 277(9):6864-73 (2002); Drebin et al., Cell, 41(3):697-706 (1985); Drebin et al., Proc. Natl. Acad. Sci. USA, 83(23):9129-33 (1986).

The term “tumor-cell killing” by anti-SALPR or anti-Relaxin-3 blocking antibodies herein is meant any inhibition of tumor cell proliferation by means of blocking a function or binding to block a pathway related to tumor-cell proliferation. For example, anti-epidermal growth factor receptor monoclonal antibodies inhibit A431 tumor cell proliferation by blocking an autocrine pathway. See Mendelsohn et al., Trans Assoc Am Physicians, 100:173-8 (1987); Masui et al., Cancer Res, 44(3):1002-7 (1984).

The term “SALPR- or Relaxin-3-oncogenic function-blocking antibody” herein is meant an anti-human SALPR- or Relaxin-3-antibody whose interaction with the SALPR or Relaxin-3 protein inhibits the oncogenic function or anti-apoptotic activity of the protein, mediates tumor-cell killing mechanisms, or inhibits tumor-cell proliferation. In contrast to antibodies that merely bind to tumor cells expressing SALPR or Relaxin-3, blocking antibodies against SALPR or Relaxin-3 mediate tumor-cell killing by mechanisms related to the oncogenic function or anti-apoptotic activity of SALPR or Relaxin-3. See Drebin et al., Proc. Natl. Acad. Sci. USA, 83(23):9129-33 (1986) for inhibition of tumorigenic growth; and Mendelsohn et al., Trans Assoc Am Physicians, 100:173-8 (1987), for an example of antibody-mediated anti-proliferative activity.

An “anti-SALPR” antibody is an antibody or antibody fragment that specifically binds a polypeptide encoded by a SALPR gene, mRNA, cDNA, or a subsequence thereof. Anti-SALPR antibody also includes a blocking antibody that inhibits oncogenic function or anti-apoptotic activity of SALPR. These antibodies can mediate anti-proliferative activity on tumor-cell growth.

An “anti-Relaxin-3” antibody is an antibody or antibody fragment that specifically binds a polypeptide encoded by a Relaxin-3 gene, mRNA, cDNA, or a subsequence thereof. Anti-Relaxin-3 antibody also includes a blocking antibody that inhibits oncogenic function or anti-apoptotic activity of Relaxin-3. These antibodies can mediate anti-proliferative activity on tumor-cell growth.

“Cancer Vaccines” are substances that are designed to stimulate the immune system to launch an immune response against a specific target associated with a cancer. For a general overview on immunotherapy and vaccines for cancers, see Old L. J., Scientific American, September, 1996.

Vaccines may be preventative or therapeutic. Typically, preventative vaccines (for example, the flu vaccine) generally contain parts of polypeptides that stimulate the immune system to generate cells and/or other substances (for example, antibodies) that fight the target of the vaccines. Preventative vaccines must be given before exposure, concurrent with exposure, or shortly thereafter to the target (for example, the flu virus) in order to provide the immune system with enough time to activate and make the immune cells and substances that can attack the target. Preventative vaccines stimulate an immune response that can last for years or even an individual's lifetime.

Therapeutic vaccines are used to combat existing disease. Thus, the goal of a therapeutic cancer vaccine is not just to prevent disease, but rather to stimulate the immune system to attack existing cancerous cells. Because of the many types of cancers and because it is often unpredictable who might get cancer, among other reasons, the cancer vaccines currently being developed are therapeutic. As discussed further below, due to the difficulties associated with fighting an established cancer, most vaccines are used in combination with cytokines or adjuvants that help stimulate the immune response and/or are used in conjunction with conventional cancer therapies.

The immune system must be able to tolerate normal cells and to recognize and attack abnormal cells. To the immune system, a cancer cell may be different in very small ways from a normal cell. Therefore, the immune system often tolerates cancer cells rather than attacking them, which allows the cancer to grow and spread. Therefore, cancer vaccines must not only provoke an immune response, but also stimulate the immune system strongly enough to overcome this tolerance. The most effective anti-tumor immune responses are achieved by stimulating T cells, which can recognize and kill tumor cells directly. Therefore, most current cancer vaccines try to activate T cells directly, try to enlist antigen presenting cells (APCs) to activate T cells, or both. By way of example, researchers are attempting to enhance T cell activation by altering tumor cells so molecules that are normally only on APCs are now on the tumor cell, thus enabling the molecules to give T cells a stronger activating signal than the original tumor cells, and by evaluating cytokines and adjuvants to determine which are best at calling APCs to areas they are needed.

Cancer vaccines can be made from whole tumor cells or from substances contained by the tumor (for example, antigens). For a whole cell vaccine, tumor cells are removed from a patient(s), grown in the laboratory, and treated to ensure that they can no longer multiply and are incapable of infecting the patient. When whole tumor cells are injected into a person, an immune response against the antigens on the tumor cells is generated. There are two types of whole cell cancer vaccines: 1) autologous whole cell vaccines made with a patient's own whole, inactivated tumor cells; and 2) allogenic whole cell vaccines made with another individual's whole, inactivated tumor cells (or the tumor cells from several individuals). Antigen vaccines are not made of whole cells, but of one or more antigens contained by the tumor. Some antigens are common to all cancers of a particular type, while some are unique to an individual. A few antigens are shared between tumors of different types of cancer.

Antigens in an antigen vaccine may be delivered in several ways. For example, proteins or fragments thereof from the tumor cells can be given directly as the vaccine. Nucleic acids coding for those proteins can be given (for example, RNA or DNA vaccines). Furthermore, viral vectors can be engineered so that when they infect a human cell and the cell will make and display the tumor antigen on its surface. The viral vector should be capable of infecting only a small number of human cells in order to start an immune response, but not enough to make a person sick. Viruses also can be engineered to make cytokines or to display proteins on their surface that help activate immune cells. These can be given alone or with a vaccine to help the immune response. Finally, antibodies themselves may be used as antigens in a vaccine (anti-idiotype vaccines). In this way, an antibody to a tumor antigen is administered, then the B cells make antibodies to that antibody that also recognize the tumor cells.

Cancer vaccines frequently contain components to help boost the immune response. Cytokines (for example, IL-2), which are chemical messengers that recruit other immune cells to the site of attack and help killer T cells perform their function, are frequently employed. Similarly, adjuvants, substances derived from a wide variety of sources, including bacteria, have been shown to elicit immune cells to an area where they are needed. In some cases, cytokines and adjuvants are added to the cancer vaccine mixture, in other cases they are given separately.

Cancer vaccines are most frequently developed to target tumor antigens normally expressed on the cell surface (for example, membrane-bound receptors or subparts thereof). However, cancer vaccines also may be effective against intracellular antigens that are, in a tumor-specific manner, exposed on the cell surface. Many tumor antigens are intracellular proteins that are degraded and expressed on the cell surface complexed with, for example, HLA. Frequently, it is difficult to attack these antigens with antibody therapy because they are sparsely dispersed on the cell surface. However, cancer vaccines are a viable alternative therapeutic approach.

Cancer vaccines may prove most useful in preventing cancer recurrence after surgery, radiation or chemotherapy has reduced or eliminated the primary tumor.

The term “immunoassay” is an assay that utilizes the binding interaction between an antibody and an antigen. Typically, an immunoassay uses the specific binding properties of a particular antibody to isolate, target, and/or quantify the antigen.

The phrases “specifically (or selectively) binds” to an antibody and “specifically (or selectively) immunoreactive with,” when referring to a protein or peptide, each refer to a binding reaction that is determinative of the presence of the protein in a heterogeneous population of proteins and other biologics. Thus, under designated immunoassay conditions, the specified antibodies bind to a particular protein at a level at least two times the background and do not substantially bind in a significant amount to other proteins present in the sample. Specific binding to an antibody under such conditions may require an antibody that is selected for its specificity for a particular protein. For example, antibodies raised to a particular SALPR or Relaxin-3 polypeptide can be selected to obtain only those antibodies that are specifically immunoreactive with the SALPR or Relaxin-3 polypeptide, respectively, and not with other proteins, except for polymorphic variants, orthologs, and alleles of the specific SALPR or Relaxin-3 polypeptide. In addition, antibodies raised to a particular SALPR or Relaxin-3 polypeptide ortholog can be selected to obtain only those antibodies that are specifically immunoreactive with the SALPR or Relaxin-3 polypeptide ortholog, and not with other orthologous proteins, except for polymorphic variants, mutants, and alleles of the SALPR or Relaxin-3 polypeptide ortholog. This selection may be achieved by subtracting out antibodies that cross-react with desired SALPR or Relaxin-3 molecules, as appropriate. A variety of immunoassay formats may be used to select antibodies specifically immunoreactive with a particular protein. For example, solid-phase ELISA immunoassays are routinely used to select antibodies specifically immunoreactive with a protein. See, for example, Harlow & Lane, Antibodies, A Laboratory Manual, 1988, for a description of immunoassay formats and conditions that can be used to determine specific immunoreactivity.

The phrase “selectively associates with” refers to the ability of a nucleic acid to “selectively hybridize” with another as defined supra, or the ability of an antibody to “selectively (or specifically) bind” to a protein, as defined supra.

“siRNA” refers to small interfering RNAs, which also include short hairpin RNA (shRNA) (see for example, Paddison et al., Genes & Dev. 16:948-958, 2002; Brummelkamp et al., Science, 296(5567):550-5533, 2002), that are capable of causing interference and can cause post-transcriptional silencing of specific genes in cells, for example, mammalian cells (including human cells) and in the body, for example, mammalian bodies (including humans). The phenomenon of RNA interference is described and discussed in Bass, Nature, 411:428-29, 2001; Elbashir et al., Nature, 411:494-98, 2001; and Fire et al., Nature, 391:806-11, 1998, wherein methods of making interfering RNA also are discussed. The siRNAs based upon the sequences disclosed herein (for example, GenBank Accession Nos. NM_{—}016568 and NM_{—}080864 for a SALPR and Relaxin-3 sequences, respectively) are typically less than 100 base pairs (“bps”) in length and constituency and preferably are about 30 bps or shorter, and can be made by approaches known in the art, including the use of complementary DNA strands or synthetic approaches. The siRNAs are capable of causing interference and can cause post-transcriptional silencing of specific genes in cells, for example, mammalian cells (including human cells) and in the body, for example, mammalian bodies (including humans). Exemplary siRNAs according to the invention could have up to 30 bps, 29 bps, 25 bps, 22 bps, 21 bps, 20 bps, 15 bps, 10 bps, 5 bps or any integer thereabout or therebetween. According to the invention, siRNA having different sequences but directed against SALPR or Relaxin-3 can be administered concurrently or consecutively in any proportion, including equimolar proportions.

The term “miRNA” refers to microRNA, a class of small RNA molecules or a small noncoding RNA molecules, that are capable of causing interference, inhibition of RNA translation into protein, and can cause post-transcriptional silencing of specific genes in cells, for example, mammalian cells (including human cells) and in the body, for example, mammalian bodies (including humans) (see, Zeng and Cullen, RNA, 9(1):112-123, 2003; Kidner and Martienssen Trends Genet, 19(1):13-6, 2003; Dennis C, Nature, 420(6917):732, 2002; Couzin J, Science 298(5602):2296-7, 2002). Previously, the miRNAs were known as small temporal RNAs (stRNAs) and belonged to a class of non-coding microRNAs, which have been shown to control gene expression either by repressing translation or by degrading the targeted mRNAs (see Couzin J, Science 298(5602):2296-7, 2002), which are generally 20-28 nt in length (see Finnegan et al., Curr Biol, 13(3):236-40, 2003; Ambros et al., RNA 9(3):277-279, 2003; Couzin J, Science 298(5602):2296-7, 2002). Unlike other RNAs (for example, siRNAs or shRNAs), miRNAs or stRNAs are not encoded by any microgenes, but are generated from aberrant (probably double-stranded) RNAs by an enzyme called Dicer, which cleaves double-stranded RNA into smaller pieces (see Couzin J, Science 298(5602):2296-7, 2002). According to the invention, miRNA having different sequences but directed against SALPR or Relaxin-3 can be administered concurrently or consecutively in any proportion, including equimolar proportions.

The term “transgene” refers to a nucleic acid sequence encoding, for example, one of the SALPR or Relaxin-3 polypeptides, or an antisense transcript thereto, which is partly or entirely heterologous, i.e., foreign, to the transgenic organism or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (for example, it is inserted at a location which differs from that of the natural gene or its insertion results in a knockout). A transgene can include one or more transcriptional regulatory sequences and any other nucleic acid, (for example, an intron), that may be necessary for optimal expression of a selected nucleic acid.

By “transgenic” is meant any organism that includes a nucleic acid sequence, which is inserted into a cell and becomes a part of the genome of the animal that develops from that cell. Such a transgene may be partly or entirely heterologous to the transgenic animal.

Thus, for example, substitution of the naturally occurring SALPR or Relaxin-3 gene for a gene from a second species results in an animal that produces the protein of the second species. Substitution of the naturally occurring gene for a gene having a mutation results in an animal that produces the mutated protein. A transgenic mouse, see below, expressing the human SALPR or Relaxin-3 protein can be generated by direct replacement of the mouse SALPR or Relaxin-3 subunit with the human gene. These transgenic animals can be critical for drug antagonist studies on animal models for human diseases, and for eventual treatment of disorders or diseases associated with the respective genes. Transgenic mice carrying these mutations will be extremely useful in studying this disease.

A “transgenic animal” refers to any animal, preferably a non-human mammal, that is chimeric, and is achievable with most vertebrate species. Such species include, but are not limited to, non-human mammals, including rodents, for example, mice and rats; rabbits; birds or amphibians; ovines, for example, sheep and goats; porcines, for example, pigs; and bovines, for example, cattle and buffalo; in which one or more of the cells of the animal contains heterologous nucleic acid introduced by way of human intervention, for example, by transgenic techniques well known in the art. The nucleic acid is introduced into the cell, directly or indirectly by introduction into a precursor of the cell, by way of deliberate genetic manipulation, for example, by microinjection or by infection with a recombinant virus. The term genetic manipulation does not include classical cross-breeding, or sexual fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the SALPR or Relaxin-3 proteins, for example, either agonistic or antagonistic forms. However, transgenic animals in which the recombinant SALPR or Relaxin-3 genes are silent also are contemplated. Moreover, “transgenic animal” also includes those recombinant animals in which gene disruption of one or more SALPR or Relaxin-3 genes is caused by human intervention, including both recombination and antisense techniques. The transgene can be limited to somatic cells or be placed into the germline.

Methods of obtaining transgenic animals are described in, for example, Puhler, A., Ed., Genetic Engineering of Animals, VCH Pub., 1993; Murphy and Carter, Eds., Transgenesis Techniques: Principles and Protocols (Methods in Molecular Biology, Vol. 18), 1993; and

Pinkert, C A, Ed., Transgenic Animal Technology: A Laboratory Handbook, Academic Press, 1994.

The term “knockout construct” refers to a nucleotide sequence that is designed to decrease or suppress expression of a polypeptide encoded by an endogenous gene in one or more cells of a mammal. The nucleotide sequence used as the knockout construct is typically comprised of (1) DNA from some portion of the endogenous gene (one or more exon sequences, intron sequences, and/or promoter sequences) to be suppressed and (2) a marker sequence used to detect the presence of the knockout construct in the cell. The knockout construct can be inserted into a cell containing the endogenous gene to be knocked out. The knockout construct can then integrate with one or both alleles of an endogenous gene, for example, SALPR or Relaxin-3 gene, and such integration of the knockout construct can prevent or interrupt transcription of the full-length endogenous gene. Integration of the knockout construct into the cellular chromosomal DNA is typically accomplished via homologous recombination (i.e., regions of the knockout construct that are homologous or complementary to endogenous DNA sequences can hybridize to each other when the knockout construct is inserted into the cell; these regions can then recombine so that the knockout construct is incorporated into the corresponding position of the endogenous DNA).

A transgenic animal carrying a “knockout” of SALPR or Relaxin-3 gene, would be useful for the establishment of a non-human model for diseases involving such proteins, and to distinguish between the activities of the different SALPR or Relaxin-3 proteins in an in vivo system. “Knockout mice” refers to mice whose native or endogenous SALPR or Relaxin-3 allele or alleles have been disrupted by homologous recombination or the like and which produce no functional SALPR or Relaxin-3 of their own. Knockout mice may be produced in accordance with techniques known in the art, for example, Thomas, et al., Immunol, 163:978-84, 1999; Kanakaraj, et al., J Exp Med, 187:2073-9, 1998; or Yeh et al., Immunity, 7:715-725, 1997.

“Aptamers”: An aptamer is a peptide, a peptide-like, a nucleic acid, or a nucleic acid-like molecule that is capable of binding to a specific molecule (for example, SALPR or Relaxin-3) of interest with high affinity and specificity. An aptamer also can be a peptide or a nucleic acid molecule that mimics the three dimensional structure of active portions of the peptides or the nucleic acid molecules of the invention. (see, for example, James W., Current Opinion in Pharmacology, 1:540-546 (2001); Colas et al., Nature 380:548-550 (1996); Tuerk and Gold, Science 249:505 (1990); Ellington and Szostak, Nature 346:818 (1990)). The specific binding molecule of the invention may be a chemical mimetic; for example, a synthetic peptide aptamer or peptidomimetic. It is preferably a short oligomer selected for binding affinity and bioavailability (for example, passage across the plasma and nuclear membranes, resistance to hydrolysis of oligomeric linkages, adsorbance into cellular tissue, and resistance to metabolic breakdown). The chemical mimetic may be chemically synthesized with at least one non-natural analog of a nucleoside or amino acid (for example, modified base or ribose, designer or non-classical amino acid, D or L optical isomer). Modification also may take the form of acylation, glycosylation, methylation, phosphorylation, sulfation, or combinations thereof. Oligomeric linkages may be phosphodiester or peptide bonds; linkages comprised of a phosphorus, nitrogen, sulfur, oxygen, or carbon atom (for example, phosphorothionate, disulfide, lactam, or lactone bond); or combinations thereof. The chemical mimetic may have significant secondary structure (for example, a ribozyme) or be constrained (for example, a cyclic peptide).

“Peptide Aptamer”: A peptide aptamer is a polypeptide or a polypeptide-like molecule that is capable of binding to a specific molecule (for example, SALPR and/or Relaxin-3) of interest with high affinity and specificity. A peptide aptamer also can be a polypeptide molecule that mimics the three dimensional structure of active portions of the polypeptide molecules of the invention. A peptide-aptamer can be designed to mimic the recognition function of complementarity determining regions of immunoglobulins, for example. The aptamer can recognize different epitopes on the protein surface (for example, SALPR and/or Relaxin-3) with dissociation equilibrium constants in the nanomolar range; those inhibit the protein (for example, SALPR and/or Relaxin-3) activity. Peptide aptamers are analogous to monoclonal antibodies, with the advantages that they can be isolated together with their coding genes, that their small size facilitates solution of their structures, and that they can be designed to function inside cells.

An peptide aptamer is typically between about 3 and about 100 amino acids or the like in length. More commonly, an aptamer is between about 10 and about 35 amino acids or the like in length. Peptide-aptamers may be prepared by any known method, including synthetic, recombinant, and purification methods (James W., Current Opinion in Pharmacology, 1:540-546 (2001); Colas et al., Nature 380:548-550 (1996)).

The instant invention also provides aptamers of SALPR and Relaxin-3 peptides. In one aspect, the invention provides aptamers of isolated polypeptides comprising at least one active fragment having substantially homologous sequence of SALPR or Relaxin-3 peptides (for example, SEQ ID NO:2 or SEQ ID NO:4, respectively, or any fragment thereof). The instant aptamers are peptide molecules that are capable of binding to a protein or other molecule, or mimic the three dimensional structure of the active portion of the peptides of the invention.

“Nucleic Acid Aptamer”: A nucleic acid aptamer is a nucleic acid or a nucleic acid-like molecule that is capable of binding to a specific molecule (for example, SALPR and/or Relaxin-3) of interest with high affinity and specificity. A nucleic acid aptamer also can be a nucleic acid molecule that mimics the three dimensional structure of active portions of the nucleic acid molecules of the invention. A nucleic acid-aptamer is typically between about 9 and about 300 nucleotides or the like in length. More commonly, an aptamer is between about 30 and about 100 nucleotides or the like in length. Nucleic acid-aptamers may be prepared by any known method, including synthetic, recombinant, and purification methods (James W., Current Opinion in Pharmacology, 1:540-546 (2001); Colas et al., Nature 380:548-550 (1996)).

According to one aspect of the invention, aptamers of the instant invention include non-modified or chemically modified RNA, DNA, PNA or polynucleotides. The method of selection may be by, but is not limited to, affinity chromatography and the method of amplification by reverse transcription (RT) or polymerase chain reaction (PCR). Aptamers have specific binding regions which are capable of forming complexes with an intended target molecule in an environment wherein other substances in the same environment are not complexed to the nucleic acid.

The instant invention also provides aptamers of SALPR and Relaxin-3 polynucleotides. In another aspect, the invention provides aptamers of isolated polynucleotides comprising at least one active fragment having substantially homologous sequence of SALPR or Relaxin-3 polynucleotides (for example, SEQ ID NO:1 or SEQ ID NO:3, respectively, or any fragment thereof). The instant aptamers are nucleic acid molecules that are capable of binding to a nucleic acid or other molecule, or mimic the three dimensional structure of the active portion of the nucleic acids of the invention.

The invention also provides nucleic acids (for example, mRNA molecules) that include an aptamer as well as a coding region for a regulatory polypeptide. The aptamer is positioned in the nucleic acid molecule such that binding of a ligand to the aptamer prevents translation of the regulatory polypeptide.

“SALPR”: The term “SALPR” can refer to SALPR nucleic acid (DNA and RNA) or protein (or polypeptide), and can include its polymorphic variants, alleles, mutants, and interspecies homologs that have (i) substantial nucleotide sequence homology (for example, at least 60% identity, preferably at least 70% sequence identity, more preferably at least 80%, still more preferably at least 90% and even more preferably at least 95%) with the nucleotide sequence of the GenBank Accession No. NM_{—}016568 (protein ID. NP_{—}057652.1); or (ii) at least 65% sequence homology with the amino acid sequence of the GenBank Protein ID. NP_{—}057652.1 (SALPR); or (iii) substantial nucleotide sequence homology (for example, at least 60% identity, preferably at least 70% sequence identity, more preferably 80%, still more preferably 85%, even more preferably at least 90% or 95%) with the nucleotide sequence as set forth in SEQ ID NO:1; or (iv) substantial sequence homology with the encoded amino acid sequence (for example, SEQ ID NO:2).

SALPR polynucleotides or polypeptides are typically from a mammal including, but not limited to, human, rat, mouse, hamster, cow, pig, horse, sheep, or any mammal. A “SALPR polynucleotide” and a “SALPR polypeptide,” may be either naturally occurring, recombinant, or synthetic (for example, produced via chemical synthesis).

SALPR DNA sequence contains 1857 base pairs (see SEQ ID NO:1), encoding a protein of 469 amino acids (see SEQ ID NO:2). GenBank Accession No. for Homo sapiens SALPR: NM_{—}016568; GenBank Protein ID. NP_{—}057652.1.

According to an aspect of the present invention, it has been determined that SALPR is amplified and/or overexpressed in human cancers, including lung cancer, colon cancer, ovarian cancer, or pancreatic cancer. Human chromosome region 5p15.1-p14 is one of the most frequently amplified regions in human cancers including lung cancer, colon cancer, ovarian cancer, and pancreatic cancer. More than one gene is located in this region. In a process of characterizing one of the 5p15.1-p14 amplicons, SALPR was found amplified in human lung cancer, colon cancer, ovarian cancer, and pancreatic cancer, and other tumor samples. Studies have shown that such amplification is usually associated with aggressive histologic types. Therefore, amplification of tumor-promoting gene(s) located on 5p15.1-p14 can play an important role in the development and/or progression of cancers including lung cancer, colon cancer, ovarian cancer, and pancreatic cancer, particularly those of the invasive histology.

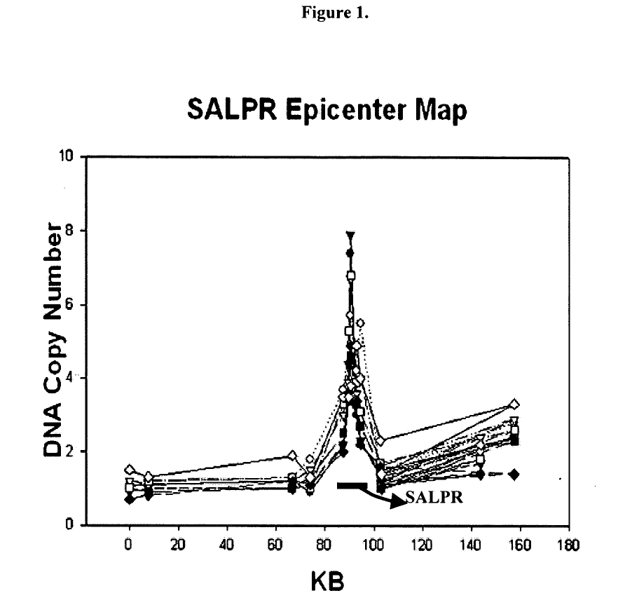

Amplification of SALPR was determined via microarray analysis (see FIG. 1). See, for example, U.S. Pat. No. 6,232,068; Pollack et al., Nat. Genet. 23(1):41-46, (1999) and other approaches known in the art. Amplified cell lines or tumors (for example, lung, colon, ovarian, and pancreatic) were examined for DNA copy number of nearby genes and DNA sequences that map to the boundaries of the amplified regions. TaqMan epicenter data for SALPR is shown in FIG. 1. Further analysis provided evidence that SALPR gene is present at the epicenter.

SALPR was found to be amplified in 16% (12/75) of lung tumors, 40% (12/30) of colon tumors, 5% (3/64) of ovarian tumors, and over 5% (1/18) of pancreatic tumors tested (see infra Table 1). SALPR was found to be overexpressed in over 6% (2/32) of lung tumors, over 88% (31/35) of colon tumors, 10% (3/30) of ovarian tumors, and over 31% (5/16) of pancreatic tumors tested (see infra Table 1).

The folds of amplification and overexpression were measured by TaqMan and RT-TaqMan, respectively, using SALPR-specific fluorogenic TaqMan probes.

“Relaxin-3”: The term “Relaxin-3” can refer to Relaxin-3 (H3) (RLN3) (also known as insulin7 (INSL7)) nucleic acid (DNA and RNA) or protein (or polypeptide), and can include its polymorphic variants, alleles, mutants, and interspecies homologs that have (i) substantial nucleotide sequence homology (for example, at least 60% identity, preferably at least 70% sequence identity, more preferably at least 80%, still more preferably at least 90% and even more preferably at least 95%) with the nucleotide sequence of the GenBank Accession No. NM_{—}080864; or (ii) at least 65% sequence homology with the amino acid sequence of the GenBank Protein ID. NP_{—}543140; or (iii) substantial nucleotide sequence homology (for example, at least 60% identity, preferably at least 70% sequence identity, more preferably 80%, still more preferably 85%, even more preferably at least 90% or 95%) with the nucleotide sequence as set forth in SEQ ID NO:3; or (iv) substantial sequence homology with the encoded amino acid sequence (for example, SEQ ID NO:4).

Relaxin-3 polynucleotides or polypeptides are typically from a mammal including, but not limited to, human, rat, mouse, hamster, cow, pig, horse, sheep, or any mammal. A “Relaxin-3 polynucleotide” and a “Relaxin-3 polypeptide,” may be either naturally occurring, recombinant, or synthetic (for example, produced via chemical synthesis).

Relaxin-3 DNA sequence contains 429 base pairs (see SEQ ID NO:3), encoding a protein of 142 amino acids (see SEQ ID NO:4). GenBank Accession No. for Homo sapiens Relaxin-3 (H3) (RLN3): NM_{—}080864; GenBank Protein ID. NP_{—}543140.

According to an aspect of the present invention, it has been determined that Relaxin-3 is amplified and/or overexpressed in human cancers, including lung cancer. Human chromosome region 19p13.2 is one of the most frequently amplified regions in human cancers including lung cancer. More than one gene is located in this region. In a process of characterizing one of the 19p13.2 amplicons, Relaxin-3 was found amplified in human lung cancer samples. Studies have shown that such amplification is usually associated with aggressive histologic types. Therefore, amplification of tumor-promoting gene(s) located on 19p13.2 can play an important role in the development and/or progression of cancers including lung cancer, particularly those of the invasive histology.

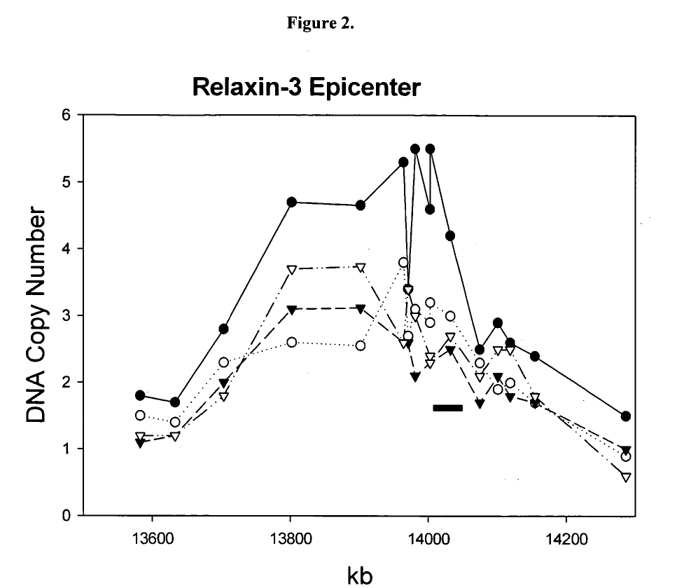

Amplification of Relaxin-3 and DNA copy numbers were determined using real time quantitative PCR (QPCR) (see FIG. 2). See, for example, U.S. Pat. No. 6,232,068; Pollack et al., Nat. Genet. 23(1):41-46, (1999) and other approaches known in the art. Amplified tumors (for example, lung tumors) were examined for DNA copy number of nearby genes and DNA sequences that map to the boundaries of the amplified regions.

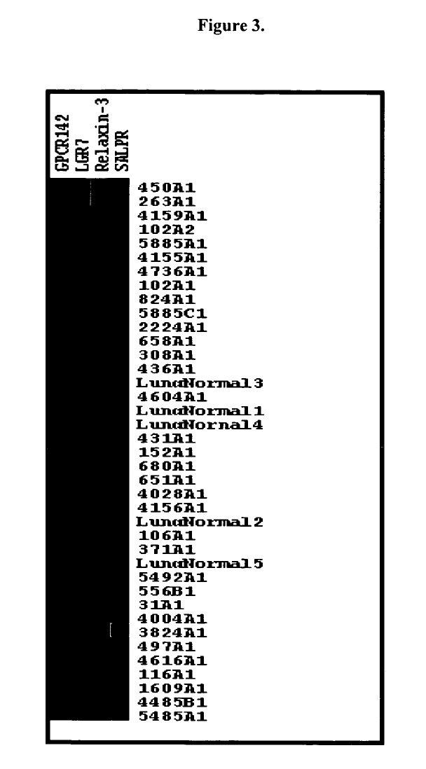

Cluster analysis of DNA copy numbers of Relaxin-3, SALPR, G protein-coupled receptor 7 (LGR7), and GPCR142 also indicate increase in DNA copy number (See FIG. 3).

Relaxin-3 was found to be amplified in 21% (7/34) of lung tumors tested (see infra Table 2). Relaxin-3 was found to be overexpressed in 15% (5/34) of lung tumors tested (see infra Table 2).

2. Amplification of SALPR and Relaxin-3 Genes in Tumors:

The presence of a target gene that has undergone amplification in tumors is evaluated by determining the copy number of the target genes, i.e., the number of DNA sequences in a cell encoding the target protein. Generally, a normal diploid cell has two copies of a given autosomal gene. The copy number can be increased, however, by gene amplification or duplication, for example, in cancer cells, or reduced by deletion. Methods of evaluating the copy number of a particular gene are well known in the art, and include, inter alia, hybridization and amplification based assays.

Any of a number of hybridization based assays can be used to detect the copy number of the SALPR or Relaxin-3 gene in the cells of a biological subject. One such method is Southern blot (see Ausubel et al., or Sambrook et al., supra), where the genomic DNA is typically fragmented, separated electrophoretically, transferred to a membrane, and subsequently hybridized to a SALPR or Relaxin-3 specific probe. Comparison of the intensity of the hybridization signal from the probe for the target region with a signal from a control probe from a region of normal nonamplified, single-copied genomic DNA in the same genome provides an estimate of the relative SALPR or Relaxin-3 gene copy number, corresponding to the specific probe used. An increased signal compared to control represents the presence of amplification.

A methodology for determining the copy number of the SALPR or Relaxin-3 gene in a sample is in situ hybridization, for example, fluorescence in situ hybridization (FISH) (see Angerer, 1987 Meth. Enzymol., 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

Another alternative methodology for determining number of DNA copies is comparative genomic hybridization (CGH). In comparative genomic hybridization methods, a “test” collection of nucleic acids is labeled with a first label, while a second collection (for example, from a normal cell or tissue) is labeled with a second label. The ratio of hybridization of the nucleic acids is determined by the ratio of the first and second labels binding to each fiber in an array. Differences in the ratio of the signals from the two labels, for example, due to gene amplification in the test collection, is detected and the ratio provides a measure of the SALPR or Relaxin-3 gene copy number, corresponding to the specific probe used. A cytogenetic representation of DNA copy-number variation can be generated by CGH, which provides fluorescence ratios along the length of chromosomes from differentially labeled test and reference genomic DNAs.

Hybridization protocols suitable for use with the methods of the invention are described, for example, in Albertson (1984) EMBO J. 3:1227-1234; Pinkel (1988) Proc. Natl. Acad. Sci. USA, 85:9138-9142; EPO Pub. No. 430:402; Methods in Molecular Biology, Vol. 33: In Situ Hybridization Protocols, Choo, ed., Humana Press, Totowa, N.J. (1994).

Amplification-based assays also can be used to measure the copy number of the SALPR or Relaxin-3 gene. In such assays, the corresponding SALPR or Relaxin-3 nucleic acid sequence act as a template in an amplification reaction (for example, Polymerase Chain Reaction or PCR). In a quantitative amplification, the amount of amplification product will be proportional to the amount of template in the original sample. Comparison to appropriate controls provides a measure of the copy number of the SALPR or Relaxin-3 gene, corresponding to the specific probe used, according to the principles discussed above. Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

A TaqMan-based assay also can be used to quantify SALPR or Relaxin-3 polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, http://www2.perkin-elmer.com).

Other suitable amplification methods include, but are not limited to, ligase chain reaction (LCR) (see, Wu and Wallace, Genomics, 4: 560, 1989; Landegren et al., Science, 241: 1077, 1988; and Barringer et al., Gene, 89:117, 1990), transcription amplification (Kwoh et al., Proc. Natl. Acad. Sci. USA, 86:1173, 1989), self-sustained sequence replication (Guatelli et al., Proc Nat Acad Sci, USA 87:1874, 1990), dot PCR, and linker adapter PCR, for example.

One powerful method for determining DNA copy numbers uses microarray-based platforms. Microarray technology may be used because it offers high resolution. For example, the traditional CGH generally has a 20 Mb limited mapping resolution; whereas in microarray-based CGH, the fluorescence ratios of the differentially labeled test and reference genomic DNAs provide a locus-by-locus measure of DNA copy-number variation, thereby achieving increased mapping resolution. Details of various microarray methods can be found in the literature. See, for example, U.S. Pat. No. 6,232,068; Pollack et al., Nat. Genet., 23(1):41-6, (1999), and others.

As demonstrated in the Examples set forth herein, the SALPR and/or Relaxin-3 genes are frequently amplified in certain cancers, particularly lung cancer, colon cancer, ovarian cancer, and pancreatic cancer. As described herein, results showing cells exhibiting a SALPR and/or Relaxin-3 DNA copy number increase also demonstrate SALPR and/or Relaxin-3 mRNA overexpression, respectively. The SALPR and Relaxin-3 genes have the characteristic features of overexpression, amplification, and the correlation between these two has been established in several tumor types. These features are shared with other well-studied oncogenes (Yoshimoto et al., JPN J Cancer Res, 77(6):540-5, 1986; Knuutila et al., Am. J. Pathol., 152(5):1107-23, 1998). The SALPR and Relaxin-3 genes and their encoded polypeptides are accordingly used in the present invention as targets for cancer diagnosis, prevention, and treatment.

3. Frequent Overexpression of SALPR and Relaxin-3 Genes in Tumors:

The expression levels of the SALPR and Relaxin-3 genes in tumors cells were examined. As demonstrated in the examples infra, SALPR and/or Relaxin-3 gene(s) is/are overexpressed in cancers, including lung cancer, colon cancer, ovarian cancer, and pancreatic cancer (See Tables 1 and 2). Detection and quantification of the SALPR or Relaxin-3 gene expression may be carried out through direct hybridization based assays or amplification based assays. The hybridization based techniques for measuring gene transcript are known to those skilled in the art (Sambrook et al., Molecular Cloning: A Laboratory Manual, 2d Ed. vol. 1-3, Cold Spring Harbor Press, NY, 1989). For example, one method for evaluating the presence, absence, or quantity of the SALPR or Relaxin-3 gene is by Northern blot. Isolated mRNAs from a given biological subject are electrophoresed to separate the mRNA species, and transferred from the gel to a membrane, for example, a nitrocellulose or nylon filter. Labeled SALPR or Relaxin-3 probes are then hybridized to the membrane to identify and quantify the respective mRNAs. The example of amplification based assays include RT-PCR, which is well known in the art (Ausubel et al., Current Protocols in Molecular Biology, eds. 1995 supplement). Quantitative RT-PCR is used preferably to allow the numerical comparison of the level of respective SALPR or Relaxin-3 mRNAs in different samples. Other assays, such as Northern hybridization or microarray analysis also can be used to determine the numerical comparison of respective mRNA levels.

4. Cancer Diagnosis, Therapies, and Vaccines Using SALPR and Relaxin-3:

A. Overexpression and Amplification of the SALPR and Relaxin-3 Genes:

The SALPR and Relaxin-3 genes and their expressed gene products can be used for diagnosis, prognosis, rational drug design, and other therapeutic intervention of tumors and cancers (for example, a lung cancer, a colon cancer, an ovarian cancer, or a pancreatic cancer).

Detection and measurement of amplification and/or overexpression of the SALPR or Relaxin-3 gene in a test sample taken from a patient indicates that the patient may have developed a tumor. Particularly, the presence of amplified SALPR or Relaxin-3 DNA leads to a diagnosis of cancer or precancerous condition, for example, a lung cancer, a colon cancer, an ovarian cancer, or a pancreatic cancer, with high probability of accuracy. The present invention therefore provides, in one aspect, methods for diagnosing, predicting, or characterizing a cancer or tumor or cancer potential in a mammalian tissue by measuring the levels of SALPR or Relaxin-3 mRNA expression in samples taken from the tissue of suspicion, and determining whether SALPR or Relaxin-3 is overexpressed in the tissue. The various techniques, including hybridization-, microarray-, and amplification-based methods, for measuring and evaluating mRNA levels are provided herein as discussed supra. The present invention also provides, in other aspects, methods for diagnosing or predicting a cancer or tumor or cancer potential in a mammalian tissue by measuring the numbers of SALPR or Relaxin-3 DNA copy in samples taken from the tissue of suspicion, and determining whether the SALPR or Relaxin-3 genes are amplified in the tissue. The various techniques, including hybridization based and amplification based methods, for measuring and evaluating DNA copy numbers are provided herein as discussed supra. The present invention thus provides methods for detecting amplified genes at the DNA level and increased expression at the RNA level, wherein both the results are indicative of tumor progression.

B. Detection of the SALPR and Relaxin-3 Protein:

According to the present invention, the detection of increased SALPR or Relaxin-3 protein level in a test sample also can indicate the presence of a precancerous or cancerous condition in the tissue source of the sample. Protein detection for tumor and cancer diagnostics and prognostics can be carried out by immunoassays, for example, using antibodies directed against a target gene, for example, SALPR and/or Relaxin-3. Any methods that are known in the art for protein detection and quantitation can be used in the methods of this invention, including, inter alia, electrophoresis, capillary electrophoresis, high performance liquid chromatography (HPLC), thin layer chromatography (TLC), hyperdiffusion chromatography, immunoelectrophoresis, radioimmunoassay (RIA), enzyme-linked immunosorbent assays (ELISAs), immuno-flouorescent assays, Western Blot, etc. Protein from the tissue or cell type to be analyzed may be isolated using standard techniques, for example, as described in Harlow and Lane, Antibodies: A Laboratory Manual (Cold Spring Harbor Laboratory Press, Cold Spring Harbor, N.Y. 1988).

The antibodies (or fragments thereof) useful in the present invention can, additionally, be employed histologically, as in immunofluorescence or immunoelectron microscopy, for in situ detection of target gene peptides. In situ detection can be accomplished by removing a histological specimen from a patient, and applying thereto a labeled antibody of the present invention. The antibody (or its fragment) is preferably applied by overlaying the labeled antibody (or fragment) onto a biological sample. Through the use of such a procedure, it is possible to determine not only the presence of the target gene product, for example, SALPR or Relaxin-3 protein, but also their distribution in the examined tissue. Using the present invention, a skilled artisan will readily perceive that any of a wide variety of histological methods (for example, staining procedures) can be modified to achieve such in situ detection.

The biological sample that is subjected to protein detection can be brought in contact with and immobilized on a solid phase support or carrier, for example, nitrocellulose, or other solid support which is capable of immobilizing cells, cell particles, or soluble proteins. The support can then be washed with suitable buffers followed by treatment with the detectably labeled fingerprint gene specific antibody. The solid phase support can then be washed with the buffer a second time to remove unbound antibody. The amount of bound label on the solid support can then be detected by conventional means.

A target gene product-specific antibody, for example, a SALPR or Relaxin-3 antibody can be detectably labeled, in one aspect, by linking the same to an enzyme, for example, horseradish peroxidase, alkaline phosphatase, or glucoamylase, and using it in an enzyme immunoassay (EIA) (see, for example, Voller, A., 1978, The Enzyme Linked Immunosorbent Assay (ELISA), Diagnostic Horizons, 2:1-7; Voller et al., J. Clin. Pathol., 31:507-520, 1978; Butler, J. E., Meth. Enzymol., 73:482-523, 1981; Maggio, E. (ed.), Enzyme Immunoassay, CRC Press, Boca Raton, Fla., 1980; and Ishikawa et al. (eds), Enzyme Immunoassay, Kgaku Shoin, Tokyo, 1981). The enzyme bound to the antibody reacts with an appropriate substrate, preferably a chromogenic substrate, in such a manner as to produce a chemical moiety that can be detected, for example, by spectrophotometric or fluorimetric means, or by visual inspection.

In a related aspect, therefore, the present invention provides the use of SALPR or Relaxin-3 antibodies in cancer diagnosis and intervention. Antibodies that specifically bind to SALPR or Relaxin-3 protein and polypeptides can be produced by a variety of methods. Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

Such antibodies can be used, for example, in the detection of the target gene, SALPR or Relaxin-3, or their fingerprint or pathway genes involved in a particular biological pathway, which may be of physiological or pathological importance. These potential pathways or fingerprint genes, for example, may interact with SALPR or Relaxin-3 activity and be involved in tumorigenesis. The SALPR or Relaxin-3 antibodies also can be used in a method for the inhibition of SALPR or Relaxin-3 activity, respectively. Thus, such antibodies can be used in treating tumors and cancers (for example, lung cancer, colon cancer, ovarian cancer, or pancreatic cancer); they also may be used in diagnostic procedures whereby patients are tested for abnormal levels of SALPR or Relaxin-3 protein, and/or fingerprint or pathway gene product associated with SALPR or Relaxin-3, respectively, and for the presence of abnormal forms of such protein.

To produce antibodies to SALPR or Relaxin-3 protein, a host animal is immunized with the protein, or a portion thereof. Such host animals can include, but are not limited to, rabbits, mice, and rats. Various adjuvants can be used to increase the immunological response, depending on the host species, including but not limited to Freund's (complete and incomplete), RIBI Detox (Ribi Immunochemical), QS21, liposomal formulations, mineral gels, for example, aluminum hydroxide, surface active substances, for example, lysolecithin, pluronic polyols, polyanions, peptides, oil emulsions, keyhole limpet hemocyanin (KLH), dinitrophenol (DNP), and potentially useful human adjuvants, for example, BCG (Bacille Calmette-Guerin) and Corynebacterium parvum.

Monoclonal antibodies, which are homogeneous populations of antibodies to a particular antigen, for example, SALPR or Relaxin-3 as in the present invention, can be obtained by any technique which provides for the production of antibody molecules by continuous cell lines in culture. These include, but are not limited to the hybridoma technique of Kohler and Milstein, (Nature, 256:495-497, 1975; and U.S. Pat. No. 4,376,110), the human B-cell hybridoma technique (Kosbor et al., Immunology Today, 4:72, 1983; Cole et al., Proc. Natl. Acad. Sci. USA, 80:2026-2030, 1983), and the BV-hybridoma technique (Cole et al., Monoclonal Antibodies And Cancer Therapy (Alan R. Liss, Inc. 1985), pp. 77-96. Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

In addition, techniques developed for the production of “chimeric antibodies” can be made by splicing the genes from a mouse antibody molecule of appropriate antigen specificity together with genes from a human antibody molecule of appropriate biological activity (see, Morrison et al., Proc. Natl. Acad. Sci. USA, 81:6851-6855, 1984; Neuberger et al., Nature, 312:604-608, 1984; Takeda et al., Nature, 314:452-454, 1985; and U.S. Pat. No. 4,816,567). A chimeric antibody is a molecule in which different portions are derived from different animal species, for example, those having a variable region derived from a murine mAb and a container region derived from human immunoglobulin.

Alternatively, techniques described for the production of single chain antibodies (for example, U.S. Pat. No. 4,946,778; Bird, Science, 242:423-426, 1988; Huston et al., Proc. Natl. Acad. Sci. USA, 85:5879-5883, 1988; and Ward et al., Nature, 334:544-546, 1989), and for making humanized monoclonal antibodies (U.S. Pat. No. 5,225,539), can be used to produce anti-differentially expressed or anti-pathway gene product antibodies.

Knappik et al. (see U.S. Pat. No. 6,300,064) describe methods for generating antibody libraries of human-derived antibody genes, which cover the antibodies encoded in the human genome. The methods disclosed also enable creation of useful libraries of (poly)peptides in general.

Antibody fragments that recognize specific epitopes can be generated by known techniques. For example, such fragments include but are not limited to: the F(ab′)₂ fragments that can be produced by pepsin digestion of the antibody molecule, and the Fab fragments that can be generated by reducing the disulfide bridges of the F(ab′)₂ fragments. Alternatively, Fab expression libraries can be constructed (Huse et al., Science, 246:1275-1281, 1989) to allow rapid and easy identification of monoclonal Fab fragments with the desired specificity.

C. Use of SALPR and Relaxin-3 Modulators in Cancer Diagnostics:

In addition to antibodies, the present invention provides, in another aspect, the diagnostic and therapeutic utilities of other molecules and compounds that interact with SALPR or Relaxin-3 protein. Specifically, such compounds can include, but are not limited to proteins or peptides, comprising extracellular portions of transmembrane proteins of the target, if they exist. Exemplary peptides include soluble peptides, for example, Ig-tailed fusion peptides. Such compounds also can be obtained through the generation and screening of random peptide libraries (see, for example, Lam et al., Nature, 354:82-84, 1991; Houghton et al., Nature, 354:84-86, 1991), made of D- and/or L-configuration amino acids, phosphopeptides (including, but not limited to, members of random or partially degenerate phosphopeptide libraries; see, for example, Songyang et al., Cell, 72:767-778, 1993), and small organic or inorganic molecules. In this aspect, the present invention provides a number of methods and procedures to assay or identify compounds that bind to target, i.e., SALPR or Relaxin-3 protein, or to any cellular protein that may interact with the target, and compounds that may interfere with the interaction of the target with other cellular proteins.

In vitro assay systems are provided that are capable of identifying compounds that specifically bind to the target gene product, for example, SALPR or Relaxin-3 protein. The assays involve, for example, preparation of a reaction mixture of the target gene product, for example, SALPR or Relaxin-3 protein and a test compound under conditions and for a time sufficient to allow the two components to interact and bind, thus forming a complex that can be removed and/or detected in the reaction mixture. These assays can be conducted in a variety of ways. For example, one method involves anchoring the target protein or the test substance to a solid phase, and detecting target protein—test compound complexes anchored to the solid phase at the end of the reaction. In one aspect of such a method, the target protein can be anchored onto a solid surface, and the test compound, which is not anchored, can be labeled, either directly or indirectly. In practice, microtiter plates can be used as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

To conduct the assay, the non-immobilized component is added to the coated surface containing the anchored component. After the reaction is complete, unreacted components are removed, for example, by washing, and complexes anchored on the solid surface are detected. Where the previously immobilized component is pre-labeled, the detection of label immobilized on the surface indicates that complexes were formed. Where the previously non-immobilized component is not pre-labeled, an indirect label can be used to detect complexes anchored on the surface; for example, using a labeled antibody specific for the immobilized component (the antibody, in turn, can be directly labeled or indirectly labeled with a labeled anti-Ig antibody). Alternatively, the reaction can be conducted in a liquid phase, the reaction products separated from unreacted components, and complexes detected, for example, using an immobilized antibody specific for a target gene or the test compound to anchor any complexes formed in solution, and a labeled antibody specific for the other component of the possible complex to detect anchored complexes.

Assays also are provided for identifying any cellular protein that may interact with the target protein, i.e., SALPR or Relaxin-3 protein. Any method suitable for detecting protein-protein interactions can be used to identify novel interactions between target protein and cellular or extracellular proteins. Those cellular or extracellular proteins may be involved in certain cancers, for example, lung cancer, colon cancer, ovarian cancer, or pancreatic cancer, and represent certain tumorigenic pathways including the target, for example, SALPR or Relaxin-3. They may thus be denoted as pathway genes.

Methods, for example, co-immunoprecipitation and co-purification through gradients or chromatographic columns, can be used to identify protein-protein interactions engaged by the target protein. The amino acid sequence of the target protein, i.e., SALPR or Relaxin-3 protein or a portion thereof, is useful in identifying the pathway gene products or other proteins that interact with SALPR or Relaxin-3 protein. The amino acid sequence of pathway gene products or other proteins can be derived from the nucleotide sequence, or from published database records (SWISS-PROT, PIR, EMBL); it also can be ascertained using techniques well known to a skilled artisan, for example, the Edman degradation technique (see, for example, Creighton, Proteins: Structures and Molecular Principles, 1983, W. H. Freeman & Co., N.Y., 34-49). The nucleotide subsequences of the target gene, for example, SALPR or Relaxin-3, can be used in a reaction mixture to screen for pathway gene sequences. Screening can be accomplished, for example, by standard hybridization or PCR techniques. Techniques for the generation of oligonucleotide mixtures and the screening are well known (see, for example, Ausubel, supra, and Innis et al. (eds.), PCR Protocols: A Guide to Methods and Applications, 1990, Academic Press, Inc., New York).

By way of example, the yeast two-hybrid system which is often used in detecting protein interactions in vivo is discussed herein. Chien et al. have reported the use of a version of the yeast two-hybrid system (Proc. Natl. Acad. Sci. USA, 1991, 88:9578-9582); it is commercially available from Clontech (Palo Alto, Calif.). Briefly, utilizing such a system, plasmids are constructed that encode two hybrid proteins: the first hybrid protein comprises the DNA-binding domain of a transcription factor, for example, activation protein, fused to a known protein, in this case, a protein known to be involved in a tumor or cancer, and the second hybrid protein comprises the activation domain of the fused transcription factor to an unknown protein that is encoded by a cDNA which has been recombined into this plasmid as part of a cDNA library. The plasmids are transformed into a strain of the yeast Saccharomyces cerevisiae that contains a reporter gene, for example, lacZ, whose expression is regulated by the transcription factor's binding site. Either hybrid protein alone cannot activate transcription of the reporter gene. The DNA binding hybrid protein cannot activate transcription because it does not provide the activation domain function, and the activation domain hybrid protein cannot activate transcription because it lacks the domain required for binding to its target site, i.e., it cannot localize to the transcription activator protein's binding site. Interaction between the DNA binding hybrid protein and the library encoded protein reconstitutes the functional transcription factor and results in expression of the reporter gene, which is detected by an assay for the reporter gene product.

The two-hybrid system or similar methods can be used to screen activation domain libraries for proteins that interact with a known “bait” gene product. The SALPR or Relaxin-3 gene product, involved in a number of tumors and cancers, is such a bait according to the present invention. Total genomic or cDNA sequences are fused to the DNA encoding an activation domain. This library and a plasmid encoding a hybrid of the bait gene product, i.e., SALPR or Relaxin-3 protein or polypeptides, fused to the DNA-binding domain are co-transformed into a yeast reporter strain, and the resulting transformants are screened for those that express the reporter gene. For example, the bait gene SALPR or Relaxin-3 can be cloned into a vector such that it is translationally fused to the DNA encoding the DNA-binding domain of the GAL4 protein. The colonies are purified and the plasmids responsible for reporter gene expression are isolated. The inserts in the plasmids are sequenced to identify the proteins encoded by the cDNA or genomic DNA.

A cDNA library of a cell or tissue source that expresses proteins predicted to interact with the bait gene product, for example, SALPR or Relaxin-3, can be made using methods routinely practiced in the art. According to the particular system described herein, the library is generated by inserting the cDNA fragments into a vector such that they are translationally fused to the activation domain of GAL4. This library can be cotransformed along with the bait gene-GAL4 fusion plasmid into a yeast strain which contains a lacZ gene whose expression is controlled by a promoter which contains a GAL4 activation sequence. A cDNA encoded protein, fused to GAL4 activation domain, that interacts with the bait gene product will reconstitute an active GAL4 transcription factor and thereby drive expression of the lacZ gene. Colonies that express lacZ can be detected by their blue color in the presence of X-gal. Plasmids from such a blue colony can then be purified and used to produce and isolate the SALPR- or Relaxin-3-interacting protein using techniques routinely practiced in the art.

The assay systems involve, for example, preparation of a reaction mixture containing the target gene product SALPR or Relaxin-3 protein, and the binding partner under conditions and for a time sufficient to allow the two products to interact and bind, thus forming a complex. To test a compound for inhibitory activity, the reaction mixture is prepared in the presence and absence of the test compound. The test compound can be initially included in the reaction mixture, or can be added at a time subsequent to the addition of a target gene product and its cellular or extracellular binding partner. Control reaction mixtures are incubated without the test compound or with a placebo. The formation of complexes between the target gene product SALPR or Relaxin-3 protein and the cellular or extracellular binding partner is then detected. The formation of a complex in the control reaction, but not in the reaction mixture containing the test compound, indicates that the compound interferes with the interaction of the target gene product SALPR or Relaxin-3 protein and the interactive binding partner. Additionally, complex formation within reaction mixtures containing the test compound and normal target gene product can be compared to complex formation within reaction mixtures containing the test compound and mutant target gene product. This comparison can be important in the situation where it is desirable to identify compounds that disrupt interactions of mutant but not normal target gene product.

The assays can be conducted in a heterogeneous or homogeneous format. Heterogeneous assays involve anchoring either the target gene product SALPR or Relaxin-3 protein or the binding partner to a solid phase and detecting complexes anchored to the solid phase at the end of the reaction, as described above. In homogeneous assays, the entire reaction is carried out in a liquid phase, as described below. In either approach, the order of addition of reactants can be varied to obtain different information about the compounds being tested. For example, test compounds that interfere with the interaction between the target gene product SALPR or Relaxin-3 protein and the binding partners, for example, by competition, can be identified by conducting the reaction in the presence of the test substance; i.e., by adding the test substance to the reaction mixture prior to or simultaneously with the target gene product SALPR or Relaxin-3 protein and interactive cellular or extracellular binding partner. Alternatively, test compounds that disrupt preformed complexes, for example, compounds with higher binding constants that displace one of the components from the complex, can be tested by adding the test compound to the reaction mixture after complexes have been formed.

In a homogeneous assay, a preformed complex of the target gene product and the interactive cellular or extracellular binding partner product is prepared in which either the target gene products or their binding partners are labeled, but the signal generated by the label is quenched due to complex formation (see, for example, Rubenstein, U.S. Pat. No. 4,109,496). The addition of a test substance that competes with and displaces one of the species from the preformed complex will result in the generation of a signal above background. The test substances that disrupt the interaction between the target gene product SALPR or Relaxin-3 protein and cellular or extracellular binding partners can thus be identified.

In one aspect, the target gene product SALPR or Relaxin-3 protein can be prepared for immobilization using recombinant DNA techniques. For example, the target SALPR or Relaxin-3 coding region can be fused to a glutathione-S-transferase (GST) gene using a fusion vector, for example, pGEX-5X-1, in such a manner that its binding activity is maintained in the resulting fusion product. The interactive cellular or extracellular binding partner product is purified and used to raise a monoclonal antibody, using methods routinely practiced in the art. This antibody can be labeled with the radioactive isotope ¹²⁵I, for example, by methods routinely practiced in the art.

In a heterogeneous assay, the GST-Target gene fusion product is anchored, for example, to glutathione-agarose beads. The interactive cellular or extracellular binding partner is then added in the presence or absence of the test compound in a manner that allows interaction and binding to occur. At the end of the reaction period, unbound material is washed away, and the labeled monoclonal antibody can be added to the system and allowed to bind to the complexed components. The interaction between the target gene product SALPR or Relaxin-3 protein and the interactive cellular or extracellular binding partner is detected by measuring the corresponding amount of radioactivity that remains associated with the glutathione-agarose beads. A successful inhibition of the interaction by the test compound will result in a decrease in measured radioactivity. Alternatively, the GST-target gene fusion product and the interactive cellular or extracellular binding partner can be mixed together in liquid in the absence of the solid glutathione-agarose beads. The test compound is added either during or after the binding partners are allowed to interact. This mixture is then added to the glutathione-agarose beads and unbound material is washed away. Again, the extent of inhibition of the binding partner interaction can be detected by adding the labeled antibody and measuring the radioactivity associated with the beads.

In other aspects of the invention, these same techniques are employed using peptide fragments that correspond to the binding domains of the target gene product, for example, SALPR or Relaxin-3 protein and the interactive cellular or extracellular binding partner (where the binding partner is a product), in place of one or both of the fill-length products. Any number of methods routinely practiced in the art can be used to identify and isolate the protein's binding site. These methods include, but are not limited to, mutagenesis of one of the genes encoding one of the products and screening for disruption of binding in a co-immunoprecipitation assay.

Additionally, compensating mutations in the gene encoding the second species in the complex can be selected. Sequence analysis of the genes encoding the respective products will reveal mutations that correspond to the region of the product involved in interactive binding. Alternatively, one product can be anchored to a solid surface using methods described above, and allowed to interact with and bind to its labeled binding partner, which has been treated with a proteolytic enzyme, for example, trypsin. After washing, a short, labeled peptide comprising the binding domain can remain associated with the solid material, which can be isolated and identified by amino acid sequencing. Also, once the gene coding for the cellular or extracellular binding partner product is obtained, short gene segments can be engineered to express peptide fragments of the product, which can then be tested for binding activity and purified or synthesized.

D. Methods for Cancer Treatment Using SALPR and Relaxin-3 Modulators:

In another aspect, the present invention provides methods for treating or controlling a cancer or tumor and the symptoms associated therewith. Any compounds, for example, those identified in the aforementioned assay systems, can be tested for the ability to prevent and/or ameliorate symptoms of tumors and cancers (for example, lung cancer, colon cancer, ovarian cancer, or pancreatic cancer). As used herein, inhibit, control, ameliorate, prevent, treat, and suppress collectively and interchangeably mean stopping or slowing cancer formation, development, or growth and/or eliminating or reducing cancer symptoms. Cell-based and animal model-based trial systems for evaluating the ability of the tested compounds to prevent and/or ameliorate tumors and cancer symptoms are used according to the present invention.

For example, cell based systems can be exposed to a compound suspected of ameliorating lung, colon, ovary, or pancreas tumor or cancer symptoms, at a sufficient concentration and for a time sufficient to elicit such an amelioration in the exposed populations of cells. After exposure, the populations of cells are examined to determine whether one or more tumor/cancer phenotypes represented in the populations has been altered to resemble a more normal or more wild-type, non-cancerous phenotype. Further, the levels of SALPR or Relaxin-3 mRNA expression and DNA amplification within these cells may be determined, according to the methods provided herein. A decrease in the observed level of expression and amplification would indicate the successful intervention of tumors and cancers (for example, lung cancer, colon cancer, ovarian cancer, or pancreatic cancer).

In addition, animal models can be used to identify compounds for use as drugs and pharmaceuticals that are capable of treating or suppressing symptoms of tumors and cancers. For example, animal models can be exposed to a test compound at a sufficient concentration and for a time sufficient to elicit such an amelioration in the exposed animals. The response of the animals to the exposure can be monitored by assessing the reversal of symptoms associated with the tumor or cancer, or by evaluating the changes in DNA copy number in cell populations and levels of mRNA expression of the target gene, for example, SALPR or Relaxin-3. Any treatments which reverse any symptom of tumors and cancers, and/or which reduce overexpression and amplification of the target SALPR or Relaxin-3 gene may be considered as candidates for therapy in humans. Dosages of test agents can be determined by deriving dose-response curves.

Moreover, fingerprint patterns or gene expression profiles can be characterized for known cell states, for example, normal or known pre-neoplastic, neoplastic, or metastatic states, within the cell- and/or animal-based model systems. Subsequently, these known fingerprint patterns can be compared to ascertain the ability of a test compound to modify such fingerprint patterns, and to cause the pattern to more closely resemble that of a normal fingerprint pattern. For example, administration of a compound which interacts with and affects SALPR or Relaxin-3 gene expression and amplification or cells overexpressing or having amplification may cause the fingerprint pattern of a precancerous or cancerous model system to more closely resemble a control, normal system; such a compound thus will have therapeutic utilities in treating the cancer. In other situations, administration of a compound may cause the fingerprint pattern of a control system to begin to mimic tumors and cancers (for example, lung cancer, colon cancer, ovarian cancer, or pancreatic cancer); such a compound therefore acts as a tumorigenic agent, which in turn can serve as a target for therapeutic interventions of the cancer and its diagnosis.

In another aspect, the present invention also provides assays for compounds that interfere with gene and cellular protein interactions involving the target SALPR or Relaxin-3. The target gene product, for example, SALPR or Relaxin-3 protein, may interact in vivo with one or more cellular or extracellular macromolecules, for example, proteins and nucleic acid molecules. Such cellular and extracellular macromolecules are referred to as “binding partners.” Compounds that disrupt such interactions can be used to regulate the activity of the target gene product, for example, SALPR or Relaxin-3 protein, especially mutant target gene product. Such compounds can include, but are not limited to, molecules, for example, antibodies, peptides and other chemical compounds.

E. Methods for Identifying Small Molecules That Can be Used as SALPR and/or Relaxin-3 Modulators:

As described herein, the modulators contemplated by the present invention can be small organic compounds. Such modulators can be identified by assays (for example, in microtiter formats on microtiter plates in robotic assays) used to screen large numbers of compounds. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In particular, modulators displaying a desired activity can be identified from combinatorial libraries (i.e., collections of diverse chemical compounds generated by either chemical synthesis or biological synthesis by combining a number of “building blocks”). Preparation and screening of combinatorial libraries is well known to those of skill in the art. Such combinatorial libraries include, but are not limited to, peptide libraries (see, for example, U.S. Pat. No. 5,010,175, Furka, Int. J. Pept. Prot. Res. 37:487-493 (1991) and Houghton et al., Nature 354:84-88 (1991)). Other chemistries for generating chemical diversity libraries also can be used. Such chemistries include, but are not limited to: peptoids (see, for example, PCT Publication No. WO 91/19735), encoded peptides (e.g., PCT Publication WO 93/20242), random bio-oligomers (e.g., PCT Publication No. WO 92/00091), benzodiazepines (see, for example, U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, for example, Ausubel, Berger and Sambrook, all supra), peptide nucleic acid libraries (see, for example, U.S. Pat. No. 5,539,083), antibody libraries (see, for example, Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, for example, benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, for example, 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are commercially available (see, for example, ComGenex, Princeton, N.J., Tripos, Inc., St. Louis, Mo., 3D Pharmaceuticals, Exton, Pa., Martek Biosciences, Columbia, Md., etc.).

High-throughput assays also can be used to identify the modulators. Using the high-throighput assays, it is possible to screen thousands of potential modulators in a single day. For example, each well of a microtiter plate can be used to run a separate assay against a selected potential modulator, or, if concentration or incubation time effects are to be observed, every 5-10 wells can test a single modulator. Thus, a single standard microtiter plate can assay about 100 (for example, 96) modulators. If 1536 well plates are used, then a single plate easily can assay from about 100- about 1500 different compounds.

F. Methods for Monitoring Efficacy of Cancer Treatment:

In one aspect, the present invention provides methods for monitoring the efficacy, such as potency, of a therapeutic treatment regimen of cancer and methods for monitoring the efficacy, such as potency, of a compound in clinical trials or other research studies for inhibition of tumors. The monitoring can be accomplished by detecting and measuring, in the biological samples taken from a patient at various time points during the course of the application of a treatment regimen for treating a cancer or a clinical trial or other research studies, the changed levels of expression or amplification of the target gene, for example, SALPR or Relaxin-3 in the cell population or sample. A level of expression and/or amplification that is lower in samples taken at the later time of the treatment or trial or a research study than those at the earlier time indicates that the treatment regimen is effective to control the cancer in the patient, or the compound is effective in inhibiting the tumor. In contrast, samples taken at the later time of the treatment or trial or a research study showing no statistically significant decrease in level of expression and/or amplification than those at the earlier time indicates that the treatment regimen is not effective to control the cancer in the patient, or the compound is not effective in inhibiting the tumor. Of course, the time course studies should be so designed that sufficient time is allowed for the treatment regimen or the compound to exert any effect it may have.

Therefore, the influence of compounds on tumors and cancers can be monitored both in a clinical trial or other research studies and in a basic drug screening. In a clinical trial or other research studies, for example, tumor cells can be isolated from lung, colon, ovary, or pancreas tumor removed by surgery, and RNA prepared and analyzed by Northern blot analysis or TaqMan RT-PCR as described herein, or alternatively by measuring the amount of protein produced. The fingerprint expression profiles thus generated can serve as putative biomarkers for lung, colon, ovary, or pancreas tumor or cancer. Particularly, the expression of SALPR or Relaxin-3 serves as one such biomarker. Thus, by monitoring the level of expression of the differentially or over-expressed genes, for example, SALPR or Relaxin-3, an effective treatment protocol can be developed using suitable chemotherapeutic anticancer drugs.

G. Use of Additional Modulators to SALPR or Relaxin-3 Nucleotides in Cancer Treatment:

In another further aspect of this invention, additional compounds and methods for treatment of tumors are provided. Symptoms of tumors and cancers can be controlled by, for example, target gene modulation, and/or by a depletion of the precancerous or cancerous cells. Target gene modulation can be of a negative or positive nature, depending on whether the target resembles a gene (for example, tumorigenic) or a tumor suppressor gene (for example, tumor suppressive). That is, inhibition, i.e., a negative modulation, of an oncogene-like target gene or stimulation, i.e., a positive modulation, of a tumor suppressor-like target gene will control or ameliorate the tumor or cancer in which the target gene is involved. More precisely, “negative modulation” refers to a reduction in the level and/or activity of target gene or its product, for example, SALPR or Relaxin-3, relative to the level and/or activity of the target gene or its product in the absence of the modulatory treatment. “Positive modulation” refers to an increase in the level and/or activity of target gene or its product, for example, SALPR or Relaxin-3, relative to the level and/or activity of target gene or its product in the absence of modulatory treatment. Particularly because SALPR or Relaxin-3 shares many features with well known oncogenes as discussed supra, inhibition of the SALPR or Relaxin-3, their proteins, or their activities will control or ameliorate precancerous or cancerous conditions, for example, lung cancer, colon cancer, ovarian cancer, or pancreatic cancer.

The techniques to inhibit or suppress a target gene, for example SALPR or Relaxin-3, that are involved in cancer are provided in the present invention. Such approaches include negative modulatory techniques. For example, compounds that exhibit negative modulatory activity on SALPR or Relaxin-3 can be used in accordance with the invention to prevent and/or ameliorate symptoms of tumors and cancers (for example, lung cancer, colon cancer, ovarian cancer, or pancreatic cancer). Such molecules can include, but are not limited to, peptides, phosphopeptides, small molecules (molecular weight below about 500 Daltons), large molecules (molecular weight above about 500 Daltons), or antibodies (including, for example, polyclonal, monoclonal, humanized, anti-idiotypic, chimeric or single chain antibodies, and Fab, F(ab′)₂ and Fab expression library fragments, and epitope-binding fragments thereof), and nucleic acid molecules that interfere with replication, transcription, or translation of the SALPR or Relaxin-3 gene (for example, antisense RNA, Antisense DNA, DNA decoy or decoy molecule, siRNAs, miRNA, triple helix forming molecules, and ribozymes, which can be administered in any combination).

Antisense, siRNAs, miRNAs, and ribozyme molecules that inhibit expression of a target gene, for example, SALPR or Relaxin-3, can be used to reduce the level of the functional activities of the target gene and its product, for example, reduce the catalytic potency of SALPR or Relaxin-3, respectively. Triple helix forming molecules can be used in reducing the level of target gene activity. These molecules can be designed to reduce or inhibit either wild type, or if appropriate, mutant target gene activity.

For example, anti-sense RNA and DNA molecules act to directly block the translation of mRNA by hybridizing to targeted mRNA and preventing protein translation. With respect to antisense DNA or DNA decoy, oligodeoxyribonucleotides derived from the translation initiation site, for example, between the −10 and +10 regions of the target gene nucleotide sequence of interest, are preferred.

Ribozymes are enzymatic RNA molecules capable of catalyzing the specific cleavage of RNA. A review is provided in Rossi, Current Biology, 4:469-471 (1994). The mechanism of ribozyme action involves sequence-specific hybridization of the ribozyme molecule to complementary target RNA, followed by an endonucleolytic cleavage. A composition of ribozyme molecules must include one or more sequences complementary to the target gene mRNA, and must include a well-known catalytic sequence responsible for mRNA cleavage (U.S. Pat. No. 5,093,246). Engineered hammerhead motif ribozyme molecules that may specifically and efficiently catalyze internal cleavage of RNA sequences encoding target protein, for example, SALPR or Relaxin-3, may be used according to this invention in cancer intervention.

Specific ribozyme cleavage sites within any potential RNA target are initially identified by scanning the molecule of interest, for example, SALPR or Relaxin-3 RNA, for ribozyme cleavage sites which include the following sequences, GUA, GUU and GUC. Once identified, short RNA sequences of between 15 and 20 ribonucleotides corresponding to the region of the target gene, for example, SALPR or Relaxin-3, containing the cleavage site can be evaluated for predicted structural features, for example, secondary structure, that can render an oligonucleotide sequence unsuitable. The suitability of candidate sequences also can be evaluated by testing their accessibility to hybridization with complementary oligonucleotides, using ribonuclease protection assays.

The SALPR or Relaxin-3 gene sequences also can be employed in an RNA interference context. The phenomenon of RNA interference is described and discussed in Bass, Nature, 411: 428-29 (2001); Elbashir et al., Nature, 411: 494-98 (2001); and Fire et al., Nature, 391: 806-11 (1998), where methods of making interfering RNA also are discussed. The double-stranded RNA based upon the sequence disclosed herein (for example, GenBank Accession No. NM_{—}016568 (SEQ ID NO:1) and NM_{—}080864 (SEQ ID NO:3) for SALPR and Relaxin-3, respectively) is typically less than 100 base pairs (“bps”) in length and constituency and preferably is about 30 bps or shorter, and can be made by approaches known in the art, including the use of complementary DNA strands or synthetic approaches. The RNAs that are capable of causing interference can be referred to as small interfering RNAs (siRNAs), small hairpin RNAs (shRNAs), or microRNAs (miRNAs), and can cause post-transcriptional silencing of specific genes in cells, for example, mammalian cells (including human cells) and in the body, for example, mammalian bodies (including humans). Exemplary siRNAs according to the invention could have up to 30 bps, 29 bps, 25 bps, 22 bps, 21 bps, 20 bps, 15 bps, 10 bps, 5 bps or any number thereabout or therebetween.

Nucleic acid molecules that can associate together in a triple-stranded conformation (triple helix) and that thereby can be used to inhibit transcription of a target gene, should be single helices composed of deoxynucleotides. The base composition of these oligonucleotides must be designed to promote triple helix formation via Hoogsteen base pairing rules, which generally require sizeable stretches of either purines or pyrimidines on one strand of a duplex. Nucleotide sequences can be pyrimidine-based, which will result in TAT and CGC triplets across the three associated strands of the resulting triple helix. The pyrimidine-rich molecules provide bases complementary to a purine-rich region of a single strand of the duplex in a parallel orientation to that strand. In addition, nucleic acid molecules can be chosen that are purine-rich, for example, those that contain a stretch of G residues. These molecules will form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation can be increased by creating a so-called “switchback” nucleic acid molecule. Switchback molecules are synthesized in an alternating 5′-3′, 3′-5′ manner, such that they base pair first with one strand of a duplex and then the other, eliminating the necessity for a sizeable stretch of either purines or pyrimidines on one strand of a duplex.

In instances wherein the antisense, ribozyme, siRNA, miRNA, and triple helix molecules described herein are used to reduce or inhibit mutant gene expression, it is possible that they also can effectively reduce or inhibit the transcription (for example, using a triple helix) and/or translation (for example, using antisense or ribozyme molecules) of mRNA produced by the normal target gene allele. These situations are pertinent to tumor suppressor genes whose normal levels in the cell or tissue need to be maintained while a mutant is being inhibited. To do this, nucleic acid molecules which are resistant to inhibition by any antisense, ribozyme or triple helix molecules used, and which encode and express target gene polypeptides that exhibit normal target gene activity, can be introduced into cells via gene therapy methods. Alternatively, when the target gene encodes an extracellular protein, it may be preferable to co-administer normal target gene protein into the cell or tissue to maintain the requisite level of cellular or tissue target gene activity. By contrast, in the case of oncogene-like target genes, for example, SALPR or Relaxin-3, it is the respective normal wild type SALPR or Relaxin-3 gene and their proteins that need to be suppressed. Thus, any mutant or variants that are defective in SALPR or Relaxin-3 function or that interferes or completely abolishes its normal function would be desirable for cancer treatment. Therefore, the same methodologies described above to safeguard normal gene alleles may be used in the present invention to safeguard the mutants of the target gene in the application of antisense, ribozyme, and triple helix treatment.

Antisense RNA and DNA or DNA decoy, ribozyme, and triple helix molecules of the invention can be prepared by standard methods known in the art for the synthesis of DNA and RNA molecules. These include techniques for chemically synthesizing oligodeoxyribonucleotides and oligoribonucleotides well known in the art, for example, solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which also include suitable RNA polymerase promoters, for example, the T7 or SP6 polymerase promoters. Alternatively, antisense cDNA constructs that synthesize antisense RNA constitutively or inducibly, depending on the promoter used, can be introduced stably into cell lines. Various well-known modifications to the DNA molecules can be introduced as a means for increasing intracellular stability and half-life. Possible modifications include, but are not limited to, the addition of flanking sequences of ribo- or deoxy- nucleotides to the 5′ and/or 3′ ends of the molecule, or the use of phosphorothioate or 2′ O-methyl rather than phosphodiesterase linkages within the oligodeoxyribonucleotide backbone.

In this aspect, the present invention also provides negative modulatory techniques using antibodies. Antibodies can be generated which are both specific for a target gene product and which reduce target gene product activity; they can be administered when negative modulatory techniques are appropriate for the treatment of tumors and cancers, for example, in the case of SALPR or Relaxin-3 antibodies for lung cancer, colon cancer, ovarian cancer, or pancreatic cancer treatment.

In instances where the target gene protein to which the antibody is directed is intracellular, and whole antibodies are used, internalizing antibodies are preferred. However, lipofectin or liposomes can be used to deliver the antibody, or a fragment of the Fab region which binds to the target gene epitope, into cells. Where fragments of an antibody are used, the smallest inhibitory fragment which specifically binds to the binding domain of the protein is preferred. For example, peptides having an amino acid sequence corresponding to the domain of the variable region of the antibody that specifically binds to the target gene protein can be used. Such peptides can be synthesized chemically or produced by recombinant DNA technology using methods well known in the art (for example, see Creighton, 1983, supra; and Sambrook et al., 1989, supra). Alternatively, single chain neutralizing antibodies that bind to intracellular target gene product epitopes also can be administered. Such single chain antibodies can be administered, for example, by expressing nucleotide sequences encoding single-chain antibodies within the target cell population by using, for example, techniques, for example, those described in Marasco et al., Proc. Natl. Acad. Sci. USA, 90:7889-7893 (1993). When the target gene protein is extracellular, or is a transmembrane protein, any of the administration techniques known in the art which are appropriate for peptide administration can be used to effectively administer inhibitory target gene antibodies to their site of action. The methods of administration and pharmaceutical preparations are discussed below.

H. Cancer Vaccines Using SALPR and Relaxin-3:

One aspect of the invention relates to methods for inducing an immunological response in a mammal which comprises inoculating the mammal with SALPR and/or Relaxin-3 polypeptide, or a fragment thereof, adequate to produce antibody and/or T cell immune response to protect the mammal from cancers, including lung cancer, colon cancer, ovarian cancer, or pancreatic cancer.

In another aspect, the invention relates to peptides derived from the SALPR or Relaxin-3 amino acid sequence (see, for example, SEQ ID NO:2 or SEQ ID NO:4, respectively) where those skilled in the art would be aware that the peptides of the present invention, or analogs thereof, can be synthesized by automated instruments sold by a variety of manufacturers, can be commercially custom ordered and prepared, or can be expressed from suitable expression vectors as described above. The term amino acid analogs has been previously described in the specification and for purposes of describing peptides of the present invention, analogs can further include branched or non-linear peptides.

The present invention therefore provides pharmaceutical compositions comprising SALPR and/or Relaxin-3 proteins or peptides derived therefrom for use in vaccines and in immunotherapy methods. When used as vaccines to protect mammals against cancer, the pharmaceutical composition can comprise as an immunogen cell lysate from cells transfected with a recombinant expression vector or a culture supernatant containing the expressed protein. Alternatively, the immunogen is a partially or substantially purified recombinant protein or a synthetic peptide.

Vaccination can be conducted by conventional methods. For example, the immunogen can be used in a suitable diluent such as saline or water, or complete or incomplete adjuvants. Further, the immunogen may or may not be bound to a carrier to make the protein immunogenic. Examples of such carrier molecules include but are not limited to bovine serum albumin (BSA), keyhole limpet hemocyanin (KLH), tetanus toxoid, and the like. The immunogen can be administered by any route appropriate for antibody production such as intravenous, intraperitoneal, intramuscular, subcutaneous, and the like. The immunogen may be administered once or at periodic intervals until a significant titer of anti-SALPR or anti-Relaxin-3 antibody is produced. The antibody may be detected in the serum using an immunoassay.

In another aspect, the present invention provides pharmaceutical compositions comprising nucleic acid sequence capable of directing host organism synthesis of a SALPR or Relaxin-3 protein or of a peptide derived from the SALPR or Relaxin-3 protein sequence. Such nucleic acid sequence may be inserted into a suitable expression vector by methods known to those skilled in the art. Expression vectors suitable for producing high efficiency gene transfer in vivo include, but are not limited to, retroviral, adenoviral and vaccinia viral vectors. Operational elements of such expression vectors are disclosed previously in the present specification and are known to one skilled in the art. Such expression vectors can be administered, for example, intravenously, intramuscularly, subcutaneously, intraperitoneally or orally.

Another aspect of the invention relates to methods for inducing an immunological response in a mammal which comprises inoculating the mammal with naked SALPR and/or Relaxin-3 nucleic acids, or a fragment thereof, adequate to produce an immunogenic polypeptide, which in turn would induce antibodies and/or a T cell immune response to protect the mammal from cancers, including lung cancer, colon cancer, ovarian cancer, or pancreatic cancer.

Naked SALPR and/or Relaxin-3 nucleic acids, as described herein, can be administered as a vaccine via various routes, including, intramuscular, intravenous, intraperitoneal, intranasal (via mucosa), intradermal, subcutaneous (see, for example, Fynan et al. Proc Natl Acad Sci USA 90:1147811482 (1993); Molling K., J Mol Med 75:242-246 (1997)). For example, naked DNA, when injected intramuscularly, is taken up by cells, transcribed into mRNA, and expressed as protein. This protein is the actual vaccine, and it is produced by the vaccine recipient, which gives a higher chance of natural modifications and correct folding. It is presented to the immune system and induces both humoral and cellular immune responses (see, for example, Tang et al. Nature 356:152154 (1992); Molling K., J Mol Med 75:242-246 (1997)).

According to the invention, liposome encapsulated SALPR and/or Relaxin-3 nucleic acids also can be administered. For example, clinical trials or other research studies with liposome encapsulated DNA in treating melanoma illustrated that the approach is effective in gene therapy (see, for example, Nabel, J. G., et al., “Direct gene transfer with DNA-liposome complexes in melanoma: Expression, biological activity and lack of toxicity in humans”, Proc. Nat. Acad. Sci. U.S.A., 90:11307-11311 (1993)).

Whether the immunogen is a SALPR or a Relaxin-3 protein, a peptide derived therefrom or a nucleic acid sequence capable of directing host organism synthesis of SALPR or Relaxin-3 protein or peptides derived therefrom, the immunogen may be administered for either a prophylactic or therapeutic purposes. Such prophylactic use may be appropriate for, for example, individuals with a genetic predisposition to a particular cancer. When provided prophylactically, the immunogen is provided in advance of the cancer or any symptom due to the cancer. The prophylactic administration of the immunogen serves to prevent or attenuate any subsequent onset of cancer. When provided therapeutically, the immunogen is provided at, or shortly after, the onset of cancer or any symptom associated with the cancer.

The present invention further relates to a vaccine for immunizing a mammal, for example, humans, against cancer comprising SALPR or Relaxin-3 protein or an expression vector capable of directing host organism synthesis of SALPR or Relaxin-3 protein in a pharmaceutically acceptable carrier.

In addition to use as vaccines and in immunotherapy, the above compositions can be used to prepare antibodies to SALPR or Relaxin-3 protein. To prepare antibodies, a host animal is immunized using the SALPR or Relaxin-3 protein or peptides derived therefrom or aforementioned expression vectors capable of expressing SALPR or Relaxin-3 protein or peptides derived therefrom. The host serum or plasma is collected following an appropriate time interval to provide a composition comprising antibodies reactive with the virus particle. The gamma globulin fraction or the IgG antibodies can be obtained, for example, by use of saturated ammonium sulfate or DEAE Sephadex, or other techniques known to those skilled in the art. The antibodies are substantially free of many of the adverse side effects which may be associated with other drugs.

The antibody compositions can be made even more compatible with the host system by minimizing potential adverse immune system responses. This is accomplished by removing all or a portion of the Fc portion of a foreign species antibody or using an antibody of the same species as the host animal, for example, the use of antibodies from human/human hybridomas. Humanized antibodies (i.e., nonimmunogenic in a human) may be produced, for example, by replacing an immunogenic portion of a non-human antibody with a corresponding, but nonimmunogenic portion (i.e., chimeric antibodies). Such chimeric antibodies may contain the reactive or antigen binding portion of an antibody from one species and the Fc portion of an antibody (nonimmunogenic) from a different species. Examples of chimeric antibodies, include but are not limited to, non-human mammal-human chimeras, such as rodent-human chimeras, murine-human and rat-human chimeras (Cabilly et al., Proc. Natl. Acad. Sci. USA, 84:3439, 1987; Nishimura et al., Cancer Res., 47:999, 1987; Wood et al., Nature, 314:446, 1985; Shaw et al., J. Natl. Cancer Inst., 80:15553,1988). General reviews of “humanized” chimeric antibodies are provided by Morrison S., Science, 229:1202, 1985 and by Oi et al., BioTechniques, 4:214, 1986.

Alternatively, anti-SALPR and/or anti-Relaxin-3 antibodies can be induced by administering anti-idiotype antibodies as immunogen. Conveniently, a purified anti-SALPR or anti-Relaxin-3 antibody preparation prepared as described above is used to induce anti-idiotype antibody in a host animal. The composition is administered to the host animal in a suitable diluent. Following administration, usually repeated administration, the host produces anti-idiotype antibody. To eliminate an immunogenic response to the Fc region, antibodies produced by the same species as the host animal can be used or the Fc region of the administered antibodies can be removed. Following induction of anti-idiotype antibody in the host animal, serum or plasma is removed to provide an antibody composition. The composition can be purified as described above for anti-SALPR or anti-Relaxin-3 antibodies, or by affinity chromatography using anti-SALPR or anti-Relaxin-3 antibodies bound to the affinity matrix. The anti-idiotype antibodies produced are similar in conformation to the authentic SALPR or Relaxin-3 antigen and may be used to prepare vaccine rather than using a SALPR or a Relaxin-3 protein.

To induce anti-SALPR or anti-Relaxin-3 antibodies in an animal, the method of administering the SALPR or Relaxin-3 antigen can be the same as used in the case of vaccination, for example, intramuscularly, intraperitoneally, subcutaneously or the like in an effective concentration in a physiologically suitable diluent with or without adjuvant. One or more booster injections may be desirable.

For both in vivo use of antibodies to SALPR or Relaxin-3 proteins and anti-idiotype antibodies and for diagnostic use, it may be preferable to use monoclonal antibodies. Monoclonal anti-SALPR or anti-Relaxin-3 antibodies, or anti-idiotype antibodies can be produced by methods known to those skilled in the art. (Goding, J. W. 1983. Monoclonal Antibodies: Principles and Practice, Pladermic Press, Inc., New York, N.Y., pp. 56-97). To produce a human-human hybridoma, a human lymphocyte donor is selected. A donor known to have the SALPR or Relaxin-3 antigen may serve as a suitable lymphocyte donor. Lymphocytes can be isolated from a peripheral blood sample or spleen cells may be used if the donor is subject to splenectomy. Epstein-Barr virus (EBV) can be used to immortalize human lymphocytes or a human fusion partner can be used to produce human-human hybridomas. Primary in vitro immunization with peptides also can be used in the generation of human monoclonal antibodies.

I. Pharmaceutical Applications of Compounds:

The identified compounds that inhibit the expression, synthesis, and/or activity of the target gene, for example, SALPR and/or Relaxin-3 can be administered to a patient at therapeutically effective doses to prevent, treat, or control a tumor or cancer. A therapeutically effective dose refers to an amount of the compound that is sufficient to result in a measurable reduction or elimination of cancer or its symptoms.

Toxicity and therapeutic efficacy of such compounds can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from the cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the method of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

Pharmaceutical compositions for use in the present invention can be formulated by standard techniques using one or more physiologically acceptable carriers or excipients. The compounds and their physiologically acceptable salts and solvates can be formulated and administered, for example, orally, intraorally, rectally, parenterally, epicutaneously, topically, transdermally, subcutaneously, intramuscularly, intranasally, sublingually, intradurally, intraocularly, intrarespiratorally, intravenously, intraperitoneally, intrathecal, mucosally, by oral inhalation, nasal inhalation, or rectal administration, for example.

For oral administration, the pharmaceutical compositions can take the form of tablets or capsules prepared by conventional means with pharmaceutically acceptable excipients, for example, binding agents, for example, pregelatinised maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulphate. The tablets can be coated by methods well known in the art. Liquid preparations for oral administration can take the form of solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations also can contain buffer salts, flavoring, coloring, and/or sweetening agents as appropriate. Preparations for oral administration can be suitably formulated to give controlled release of the active compound.

For administration by inhalation, the compounds are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, before use. The compounds also can be formulated in rectal compositions, for example, suppositories or retention enemas, for example, containing conventional suppository bases, for example, cocoa butter or other glycerides.

Furthermore, the compounds also can be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device which can contain one or more unit dosage forms containing the active ingredient. The pack can for example comprise metal or plastic foil, for example, a blister pack. The pack or dispenser device can be accompanied by instructions for administration.

J. Administration of siRNA/shRNA/miRNA:

The invention includes methods of administering siRNA, shRNA, and miRNA, to a patient in need thereof, wherein the siRNA, shRNA, or miRNA molecule is delivered in the form of a naked oligonucleotide or via an expression vector as described herein.

The present invention provides methods of blocking the in vivo expression of SALPR or Relaxin-3 gene by administering a naked DNA or a vector containing siRNA, shRNA, or miRNA as set forth herein (see, for example, Examples VIII to XII), which interacts with the target gene and causes post-transcriptional silencing of specific genes in cells, for example, mammalian cells (including human cells) and in the body, for example, mammalian bodies (including humans).

The invention also provides methods for the treatment of cells ex vivo by administering a naked DNA or a vector according to the invention.

In its in vivo or ex vivo therapeutic applications, it is appropriate to administer siRNA, shRNA, or miRNAs using a viral or retroviral vector, which enters the cell by transfection or infection. In particular, as a therapeutic product according to the invention, a vector can be a defective viral vector, such as an adenovirus, or a defective retroviral vector, such as a murine retrovirus.

The vector used to convey the gene construct according to the invention to its target can be a retroviral vector, which will transport the recombinant construct by a borrower capsid, and insert the genetic material into the DNA of the host cell.

Techniques that use vectors, in particular viral vectors (retroviruses, adenoviruses, adeno-associated viruses), to transport genetic material to target cells can be used to introduce genetic modifications into various somatic tissues, for example, lung, colon, ovary, or pancreas cells.

The use of retroviral vectors to transport genetic material necessitates, on the one hand, carrying out the genetic construction of the recombinant retrovirus, and on the other hand having a cell system available which provides for the function of encapsidation of the genetic material to be transported:
i. In a first stage, genetic engineering techniques enable the genome of a murine retrovirus, such as Moloney virus (murine retrovirus belonging to the murine leukemia virus group (Reddy et al., Science, 214:445-450 (1981)). The retroviral genome is cloned into a plasmid vector, from which all the viral sequences coding for the structural proteins (genes: Gag, Env) as well as the sequence coding for the enzymatic activities (gene: Pol) are then deleted. As a result, only the necessary sequences “in cis” for replication, transcription and integration are retained (sequences corresponding to the two LTR regions, encapsidation signal and primer binding signal). The deleted genetic sequences may be replaced by non-viral genes such as the gene for resistance to neomycin (selection antibiotic for eukaryotic cells) and by the gene to be transported by the retroviral vector, for example, SALPR or Relaxin-3 siRNA as set forth herein.
ii. In a second stage, the plasmid construct thereby obtained is introduced by transfection into the encapsidation cells. These cells constitutively express the Gag, Pol and Env viral proteins, but the RNA coding for these proteins lacks the signals needed for its encapsidation. As a result, the RNA cannot be encapsidated to enable viral particles to be formed. Only the recombinant RNA emanating from the transfected retroviral construction is equipped with the encapsidation signal and is encapsidated. The retroviral particles produced by this system contain all the elements needed for the infection of the target cells (such as CD34+ cells) and for the permanent integration of the gene of interest into these cells, for example, SALPR or Relaxin-3 siRNA as set forth herein. The absence of the Gag, Pol and Env genes prevents the system from continuing to propagate.

DNA viruses such as adenoviruses also can be suited to this approach although, in this case, maintenance of the DNA in the episomal state in the form of an autonomous replicon is the most likely situation.

Adenoviruses possess some advantageous properties. In particular, they have a fairly broad host range, are capable of infecting quiescent cells and do not integrate into the genome of the infected cell. For these reasons, adenoviruses have already been used for the transfer of genes in vivo. To this end, various vectors derived from adenoviruses have been prepared, incorporating different genes (beta-gal, OTC, alpha-1At, cytokines, etc.). To limit the risks of multiplication and the formation of infectious particles in vivo, the adenoviruses used are generally modified so as to render them incapable of replication in the infected cell. Thus, the adenoviruses used generally have the E1 (E1a and/or E1b) and possibly E3 regions deleted.

The defective recombinant adenoviruses according to the invention may be prepared by any technique known to persons skilled in the art (Levrero et al., Gene, 101:195 (1991), EP 185 573; Graham, EMBO J. 3:2917 (1984)). In particular, they may be prepared by homologous recombination between an adenovirus and a plasmid in a suitable cell line.

According to the present invention, an exogenous DNA sequence, for example, SALPR or Relaxin-3 siRNA as set forth herein, is inserted into the genome of the defective recombinant adenovirus.

Pharmaceutical compositions comprising one or more viral vectors, such as defective recombinants as described above, may be formulated for the purpose of topical, oral, parenteral, intranasal, intravenous, intramuscular, subcutaneous, intraocular, and the like, administration. Preferably, these compositions contain vehicles which are pharmaceutically acceptable for an administrable formulation. These can be, in particular, isotonic, sterile saline solutions (of monosodium or disodium phosphate, sodium, potassium, calcium or magnesium chloride, and the like, or mixtures of such salts), or dry, in particular lyophilized, compositions which, on addition, as appropriate, of sterilized water or of physiological saline, enable particular injectable solutions to be made up.

The doses of defective recombinant virus used for the injection may be adapted in accordance with various parameters, and in particular in accordance with the mode of administration used, the pathology in question, the gene to be expressed or the desired duration of treatment. Generally speaking, the recombinant adenoviruses according to the invention may be formulated and administered in the form of doses of between 10⁴ and 10⁴ pfu/ml, and preferably 10⁶ to 10¹⁰ pfu/ml. The term pfu (“plaque forming unit”) corresponds to the infectious power of a solution of virus, and is determined by infection of a suitable cell culture and measurement, generally after 48 hours, of the number of plaques of infected cells. The techniques of determination of the pfu titer of a viral solution are well documented in the literature.

The use of genetically modified viruses as a shuttle system for transporting the modified genetic material not only permits the genetic material to enter the recipient cell by the expedient of using a borrower viral capsid, but also allows a large number of cells to be treated simultaneously and over a short period of time, which permits therapeutic treatment applied to the whole body.

The invention is further described by the following examples, which do not limit the invention in any manner.

### EXAMPLES

### Example I

### Amplification of the SALPR Gene in Human Cancers

DNA microarray-based comparative genomic hybridization (CGH) was used to survey the genome for gene amplification, and it was determined that the SALPR gene is frequently amplified in tumor tissue and cell lines.

Genomic DNAs were isolated from lung, colon, ovarian, and pancreatic tumor samples. DNAs were analyzed, along with (i) a SALPR TaqMan probe representing the target and (ii) a reference probe representing a normal non-amplified, single copy region in the genome, with a TaqMan 7900 Sequence Detector (Applied Biosystems) following the manufacturer's protocol.

SALPR gene was found to be amplified in primary lung, colon, ovarian, and pancreatic tumors samples. SALPR was found amplified in 16% (12/75) of lung tumors, 40% (12/30) of colon tumors, 5% (3/64) of ovarian tumors, and over 5% (1/18) of pancreatic tumors tested (see Table 1).

Only samples with the SALPR gene copy number greater than or equal to 3.0-fold are deemed to have been amplified because of instrumental detection limit. However, an increase in SALPR gene copy number less than 3.0-fold can still be considered as an amplification of the gene, if detected.

### Example II

### Overexpression of the SALPR in Tumors

Reverse transcriptase (RT)-directed quantitative PCR was performed using the TaqMan 7900 Sequence Detector (Applied Biosystems) to determine the SALPR mRNA level in each sample. Human β-actin mRNA was used as control.

Total RNA was isolated from tumor samples using Trizol Reagent (Invitrogen) and treated with DNAase (Ambion) to eliminate genomic DNA. The reverse transcriptase reaction (at 48° C. for 30 minutes, for example) was coupled with quantitative PCR measurement of cDNA copy number in a one-tube format according to the manufacturer (Perkin Elmer/Applied Biosystems). The nucleotide sequences of SALPR were used to design and make a suitable TaqMan probe set (see GenBank RECORD NM_{—}016568) for SALPR. SALPR expression levels in the samples were normalized using human β-actin and overexpression fold was calculated by comparing SALPR expression in tumor versus normal samples.

The RT-TaqMan showed that SALPR gene is overexpressed in primary lung, colon, ovarian, and pancreatic tumors. More specifically, SALPR was found to be overexpressed in over 6% (2/32) of lung tumors, over 88% (31/35) of colon tumors, 10% (3/30) of ovarian tumors, and over 31% (5/16) of pancreatic tumors tested (see Table 1). Cancer-free normal tissues from the above-identified source types were used as controls.

TABLE 1
Amplification and overexpression of SALPR in primary lung,
colon, ovarian, and pancreatic tumors.

| | SALPR Amplification* | | SALPR Overexpression* | |
|---|---|---|---|---|
| Tumor Type | Frequency | Highest Fold | Frequency | Highest Fold |
| Lung Tumors | 12/75 > 3X (16%) | 5.5X | 02/32 > 5X (6%) | 247X |
| Colon Tumors | 12/30 > 3X (40%) | 7.6X | 31/35 > 10X (88%) | >1000X |
| Ovarian Tumors | 03/64 > 3X (5%) | 7.5X | 03/30 > 5X (10%) | 7.2X |
| Pancreatic | 01/18 > 2.5X (5%) | 2.6X | 05/16 > 5X (3%) | 137X |
| Tumors | | | | |

*Amplification cutoff: 3.0X.
*Overexpression cutoff: 5X using β-actin as reference.

### Example III

### Physical Map of the Amplicon Containing the SALPR Gene Locus

Cancer cell lines or primary tumors were examined for DNA copy number of genes and markers near SALPR to map the boundaries of the amplified regions.

DNA was purified from tumor cell lines or primary tumors. The DNA copy number of each marker in each sample was directly measured using PCR and a fluorescence-labeled probe. The number of PCR cycles needed to cross a preset threshold, also known as Ct value, in the sample tumor DNA preparations and a series of normal human DNA preparations at various concentrations was determined for both the target probe and a known single-copy DNA probe using a TaqMan 7900 Sequence Detector (Applied Biosystems). The relative abundance of target sequence to the single-copy probe in each sample was then calculated by statistical analyses of the Ct values of the unknown samples and the standard curve was generated from the normal human DNA preparations at various concentrations.

To determine the DNA copy number for each of the genes, corresponding probes to each marker were designed using PrimerExpress 1.0 (Applied Biosystems) and synthesized by Operon Technologies. Subsequently, the target probe (representing the marker), a reference probe (representing a normal non-amplified, single copy region in the genome), and tumor genomic DNA (10 ng) were subjected to analysis by the TaqMan 7900 Sequence Detector (Applied Biosystems) following the manufacturer's protocol. The epicenter mapping around SALPR gene was performed using amplified tumor and tumor cell line samples.

Human chromosome region 5p15.1-p14 was identified initially by DNA microarray analysis of human lung tumor samples for DNA amplification. SALPR was mapped to chromosome 5p15.1-p14 by FISH method as described supra and found to be the only gene in this region. FIG. 1 shows cDNA microarray analysis of eight human lung tumor samples. The fluorescence ratios were plotted against their physical presence on human chromosome 5p15.1-p14 based on Human Genome Project Working Draft Sequences (http://genome.ucsc.edu/goldenPath/hgTracks.html). It was demonstrated that SALPR is the only gene within the amplified region (see FIG. 1). A full-length SALPR gene was present at the epicenter.

### Example IV

### Tumorigenicity of SALPR in Nude Mice

3T3 cells were engineered to express SALPR (full length untagged) or full length SALPR with an C-terminal Flag tag (“SALPR C terminal FLAG”) using retroviral transduction. As controls, the parental 3T3 cell line was untrandsduced or transduced with pLPC vector alone (“Vector”).

Cells were grown in DMEM supplemented with 10% calf serum, 2 mM L-glutamine, non essential amino acids and sodium pyruvate at 37° C., 5% CO₂, 95% humidity. Cultures were detached from culture plates using trypsin/EDTA in one tube. Cells were counted by hemocytometer, washed 1× with PBS (without Ca²⁺ or Mg²⁺) and resuspended in PBS to a final concentration of 25×10⁶ cells/mL. 5×10⁶ cells or 0.2 mL were injected subcutaneously per athymic nude mouse.

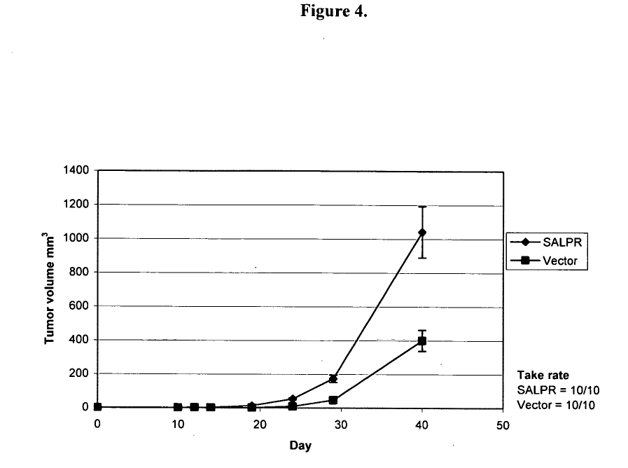

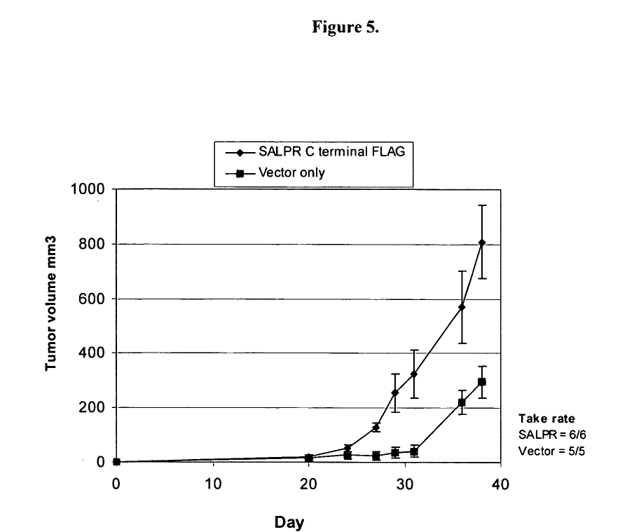

Tumor growth (Mean±SEM) in athymic nude mice following implantation of about 5 million 3T3 transfectants is shown in FIGS. 4-5. A total of 10 mice were used for each experimental/control group and palpable/measurable tumors were recorded. The mice were checked daily and when a palpable tumor was evident. Tumor volumes were measured with a caliper in three perpendicular dimensions and recorded as mm³. Results indicate that 10/10 nude mice injected with 3T3 cells containing full length SALPR with no C-terminal flag tag (full length untagged) produced significantly larger tumors (over 1000 mm³) (see FIG. 4), compared to those (5/5 or 6/6 nude mice) implanted with 3T3 parental cells (about 400 mm³), or “SALPR C terminal FLAG” (about 800 mm³) (see FIG. 5).

### Example V

### Amplification of the Relaxin-3 Gene in Human Cancers

DNA microarray-based comparative genomic hybridization (CGH) was used to survey the genome for gene amplification, and it was determined that the Relaxin-3 gene is frequently amplified in tumor tissues.

Genomic DNAs were isolated from lung tumor samples. DNAs were analyzed, along with (i) a Relaxin-3 TaqMan probe representing the target and (ii) a reference probe representing a normal non-amplified, single copy region in the genome, with a TaqMan 7900 Sequence Detector (Applied Biosystems) following the manufacturer's protocol.

Relaxin-3 gene was found to be amplified in primary lung tumors samples. Relaxin-3 was found amplified in 21% (7/34) of lung tumors tested (see Table 2). Relaxin-3 is amplified gene in lung cancer and occasionally co-amplified with its receptor, SALPR.

Only samples with the Relaxin-3 gene copy number greater than or equal to 3.0-fold are deemed to have been amplified because of instrumental detection limit. However, an increase in Relaxin-3 gene copy number less than 3.0-fold can still be considered as an amplification of the gene, if detected.

### Example VI

### Overexpression of the Relaxin-3 in Tumors

Reverse transcriptase (RT)-directed quantitative PCR was performed using the TaqMan 7900 Sequence Detector (Applied Biosystems) to determine the Relaxin-3 mRNA level in each sample. Human β-actin mRNA was used as control.

Total RNA was isolated from tumor samples using Trizol Reagent (Invitrogen) and treated with DNAase (Ambion) to eliminate genomic DNA. The reverse transcriptase reaction (at 48° C. for 30 minutes, for example) was coupled with quantitative PCR measurement of cDNA copy number in a one-tube format according to the manufacturer (Perkin Elmer/Applied Biosystems). The nucleotide sequences of Relaxin-3 were used to design and make a suitable TaqMan probe set (see GenBank RECORD NM_{—}080864) for Relaxin-3. Relaxin-3 expression levels in the samples were normalized using human β-actin and overexpression fold was calculated by comparing Relaxin-3 expression in tumor versus normal samples.

The RT-TaqMan showed that Relaxin-3 gene is overexpressed in primary lung tumors. More specifically, Relaxin-3 was found to be overexpressed in 15% (5/34) of lung tumors tested (see infra Table 2). Cancer-free normal tissues from the above-identified source types were used as controls.

TABLE 2
Amplification and overexpression of Relaxin-3
in primary lung tumors.

| | Relaxin-3 Amplification* | Relaxin-3 Overexpression* |
|---|---|---|
| Tumor Type | Frequency | Frequency |
| Lung Tumors | 7/34 > 3X (21%) | 5/34 > 5X (15%) |

*Amplification cutoff: 3.0X.
*Overexpression cutoff: 5X using β-actin as reference.

### Example VII

### Relaxin-3 Epicenter of the Genomic DNA Locus Containing SALPR

DNA copy number was determined using real time quantitative PCR (QPCR) in four lung tumor samples (450A1, 102A1, 4159A1, 5885C1). Relaxin-3 gene (solid black bar) is contained in the minimal commonly amplified region of approximately 400 kb in size, implicating that relaxin-3 is targeted by DNA copy number increase for this region (See FIG. 2).

Cluster analysis of DNA copy numbers of Relaxin-3, SALPR, G protein-coupled receptor 7 (LGR7), and GPCR142 were carried out. Results are displayed in the format of Eisen dendrogram (See FIG. 3). LGR7 and GPCR142 are two other receptors are known to have affinity for relaxin-3. Relaxin-3 and SALPR are put next to each other as being more closely related in terms of DNA copy number increase in this panel of tumors. Results indicate increase in DNA copy number, for example, tumors samples 263A1 and 4159A1 exhibit amplifications of both Relaxin-3 and SALPR (See FIG. 3, gray shades).

### Example VIII

### Small Interfering RNA (siRNA)

Sense and antisense siRNAs duplexes are made based upon targeted region of a SALPR or a Relaxin-3 DNA sequences, disclosed herein (see, for example, SEQ ID NO:1 (SALPR), SEQ ID NO:3 (Relaxin-3), or a fragment thereof), are typically less than 100 base pairs (“bps”) in length and constituency and preferably are about 30 bps or shorter, and are made by approaches known in the art, including the use of complementary DNA strands or synthetic approaches. SiRNA derivatives employing polynucleic acid modification techniques, such as peptide nucleic acids, also can be employed according to the invention. The siRNAs are capable of causing interference and can cause post-transcriptional silencing of specific genes in cells, for example, mammalian cells (including human cells) and in the body, for example, mammalian bodies (including humans). Exemplary siRNAs according to the invention have up to 30 bps, 29 bps, 25 bps, 22 bps, 21 bps, 20 bps, 15 bps, 10 bps, 5 bps or any integer thereabout or therebetween.

A targeted region is selected from the DNA sequence (for example, SEQ ID NO:1, SEQ ID NO:3, or a fragment thereof). Various strategies are followed in selecting target regions and designing siRNA oligos, for example, 5′ or 3′ UTRs and regions nearby the start codon should be avoided, as these may be richer in regulatory protein binding sites. Designed sequences preferably include AA-(N27 or less nucleotides)-TT and with about 30% to 70% G/C-content. If no suitable sequences are found, the fragment size is extended to sequences AA(N29 nucleotides). The sequence of the sense siRNA corresponds to, for example, (N27 nucleotides)-TT or N29 nucleotides, respectively. In the latter case, the 3′ end of the sense siRNA is converted to TT. The rationale for this sequence conversion is to generate a symmetric duplex with respect to the sequence composition of the sense and antisense 3′ overhangs. It is believed that symmetric 3′ overhangs help to ensure that the small interfering ribonucleoprotein particles (siRNPs) are formed with approximately equal ratios of sense and antisense target RNA-cleaving siRNPs (Elbashir et al. Genes & Dev. 15:188-200, 2001).

### Example IX

### SALPR siRNA

Sense or antisense siRNAs are designed based upon targeted regions of a DNA sequence, as disclosed herein (see, for example, SEQ ID NO:1, GenBank Accession No. NM_{—}016568), and include fragments having up to 30 bps, 29 bps, 25 bps, 22 bps, 21 bps, 20 bps, 15 bps, 10 bps, 5 bps or any integer thereabout or therebetween. For example, 29 bps siRNA include:
Targeted region (base position numbers 376-404, SEQ ID NO:5) 5′-GCAGCCACGATAGCCACCATGAATAAGGC-3′, the corresponding sense siRNA (SEQ ID NO:6) 5′-GCAGCCACGAUAGCCACCAUGAAUAAGGC-3′, and the antisense siRNA (SEQ ID NO:7) 5′-GCCUUAUUCAUGGUGGCUAUCGUGGCUGC-3′;
Targeted region (base position numbers 379-407, SEQ ID NO:8) 5′-GCCACGATAGCCACCATGAATAAGGCAGC-3′, the corresponding sense siRNA (SEQ ID NO:9) 5′-GCCACGAUAGCCACCAUGAAUAAGGCAGC-3′, and the antisense siRNA (SEQ ID NO:10) 5′-GCUGCCUUAUUCAUGGUGGCUAUCGUGGC-3′;
Targeted region (base position numbers 486-514, SEQ ID NO:11) 5′-GCTGCAGCTTCCGGACTTGTGGTGGGAGC-3′, the corresponding sense siRNA (SEQ ID NO:12) 5′-GCUGCAGCUUCCGGACUUGUGGUGGGAGC-3′, and the antisense siRNA (SEQ ID NO:13) 5′-GCUCCCACCACAAGUCCGGAAGCUGCAGC-3′;
Targeted region (base position numbers 492-520, SEQ ID NO:14) 5′-GCTTCCGGACTTGTGGTGGGAGCTGGGGC-3′, the corresponding sense siRNA (SEQ ID NO:15) 5′-GCUUCCGGACUUGUGGUGGGAGCUGGGGC-3′, and the antisense siRNA (SEQ ID NO:16) 5′-GCCCCAGCUCCCACCACAAGUCCGGAAGC-3′;
Targeted region (base position numbers 644-672, SEQ ID NO:17) 5′-GGTTGGCGGGCAACCTGCTGGTTCTCTAC-3′, the corresponding sense siRNA (SEQ ID NO:18) 5′-GGUUGGCGGGCAACCUGCUGGUUCUCUAC-3′, and the antisense siRNA (SEQ ID NO:19) 5′-GUAGAGAACCAGCAGGUUGCCCGCCAACC-3′;
Targeted region (base position numbers 645-673, SEQ ID NO:20) 5′-GTTGGCGGGCAACCTGCTGGTTCTCTACC-3′, the corresponding sense siRNA (SEQ ID NO:21) 5′-GUUGGCGGGCAACCUGCUGGUUCUCUACC-3′, and the antisense siRNA (SEQ ID NO:22) 5′-GGUAGAGAACCAGCAGGUUGCCCGCCAAC-3′; and continuing in this progression to the end of SALPR sequence, for example,
Targeted region (base position numbers 1732-1760, SEQ ID NO:23) 5′-GGGCGCTACGACCTGCTGCCCAGCAGCTC-3′, the corresponding sense siRNA (SEQ ID NO:24) 5′-GGGCGCUACGACCUGCUGCCCAGCAGCUC-3′, and the antisense siRNA (SEQ ID NO:25) 5′-GAGCUGCUGGGCAGCAGGUCGUAGCGAAA-3;
Targeted region (base position numbers 1736-1764, SEQ ID NO:26) 5′-GCTACGACCTGCTGCCCAGCAGCTCTGCC-3′, the corresponding sense siRNA (SEQ ID NO:27) 5′-GCUACGACCUGCUGCCCAGCAGCUCUGCC-3′, and the antisense siRNA (SEQ ID NO:28) 5′-GGCAGAGCUGCUGGGCAGCAGGUCGUAGC-3; and so on as set forth herein.

A set of siRNAs/shRNAs are designed based on SALPR-coding sequence (see, for example, SEQ ID NO:1, GenBank Accession No. NM_{—}016568; coding-region base positions: 361-1770).

### Example X

### A PCR-based Strategy for Cloning SALPR siRNA/shRNA Sequences

SALPR oligos can be designed based on a set criteria, for example, 29 bps ‘sense’ sequences (for example, a target region starting base position number 376 of the SALPR sequence: 5′-GCAGCCACGATAGCCACCATGAATAAGGC-3′, SEQ ID NO:5) containing a ‘C’ at the 3′ end are selected from the SALPR sequence. A termination sequence (for example, AAAAAA, SEQ ID NO:29), the corresponding antisense SALPR sequence (for example, 5′-GCCTTATTCATGGTGGCTATCGTGGCTGC-3′, SEQ ID NO:30), a loop (for example, CAAGCTTC, SEQ ID NO:31), and a reverse primer (for example, U6 reverse primer, GGTGTTTCGTCCTTTCCACAA, SEQ ID NO:32) are subsequently added to the 29 bps sense strands to construct PCR primers (see for example, Paddison et al., Genes & Dev. 16:948-958, 2002). Of course, other sense and anti-sense sequences can be selected from a target molecule to develop siRNAs for that molecule.

Several steps are followed in generating hairpin primers. First, a 29 nt “sense” sequence containing a “C” at the 3′ end is selected. Second, the actual hairpin is constructed in a 5′-3′ orientation with respect to the intended transcript. Third, a few stem pairings are changed to G-U by altering the sense strand sequence. G-U base pairing seems to be beneficial for stability of short hairpins in bacteria and does not interfere with silencing. Finally, the hairpin construct is converted to its “reverse complement” and combined with 21 nt human U6 promoter. See below, an example of the model structures drawn:

A model shRNA structure based on SEQ ID NO:5 is (SEQ ID NO:33):

```
                          5′->3′ Anti-sense strand
-------| GAA
              GCAGCCACGAUAGCCACCAUGAAUAAGGC G
              CGUCGGUGCUAUCGGUGGUACUUAUUCCG C
          UU\hat{ } GUU
                            3′<-5′ Sense strand
```

The linear form of the model (SEQ ID NO:34):

Anti-sense Loop Sense Termination
GCAGCCACGAUAGCCACCAUGAAUAAGGCGAAGCUUGGCCUUAUUCAUGGUGGCUAUCGUGGCUGCUUUUUU

Some base pairing are changed to G-U by altering sense sequence. The final hairpin is converted to its reverse complement.

Hairpin portion of the primer (about 72 nt) (SEQ ID NO:35):

AAAAAAGCAGCCACGAUAGCCACCAUGAAUAAGGCCAAGCUUCGCCUUAUUCAUGGUGGCUAUCGUGGCUGC
+

U6 promoter (reverse primer sequence): GGUGUUUCGUCCUUUCCACAA (SEQ ID NO:36)

Thus, the final hairpin sequence (SEQ ID NO:37) is:

AAAAAAGCAGCCACGAUAGCCACCAUGAAUAAGGCCAAGCUUCGCCUUAU
UCAUGGUGGCUAUCGUGGCUGCGGUGUUUCGUCCUUUCCACAA

Model shRNA structures also can be developed based on a different set of criteria (see for example, Brummelkamp et al., Science, 296(5567):550-5533, 2002). Thus, SALPR oligos, siRNA/shRNA also can be designed based sense or anti-sense sequences and the model structure.

PCR and Cloning: A pGEM1 plasmid (Promega) containing the human U6 locus (G. Hannon, CSHL) is used as the template for the PCR reaction. This vector contains about 500 bp of upstream U6 promoter sequence. Since an SP6 sequence flanks the upstream portion of the U6 promoter, an SP6 oligo is used as the universal primer in U6-hairpin PCR reactions. The PCR product is about 600 bp in length. T-A and directional topoisomerase-mediated cloning kits (Invitrogen, Inc. Catalog No. K2040-10, K2400-20) are used according to the manufacturer's instruction.

To obtain stable siRNAs/shRNAs, some nucleotide bases are modified, therefore, the designed oligo sequences may not match the actual coding sequences.

Examples of oligos designed and the targeted base position numbers of the 29 nt sense sequence of the SALPR-coding region (see, for example, SEQ ID NO:1, GenBank Accession No. NM_{—}016568; coding-region base positions: 361-1770) are shown below:
SEQ ID NO:38: Primer containing a target region (starting base position number 376 of the SALPR sequence):
AAAAAAGCAGCCACGAUAGCCACCAUGAAUAAGGCCAAGCUUCGCCUUAUUCAUGGUGGCUAUCGUGGCUGCGGUGUUUCGUCCUUUCCACAA-3′, and
the targeted SALPR-coding region is (coding region base position numbers 376-404, SEQ ID NO:5) 5′-GCAGCCACGATAGCCACCATGAATAAGGC-3′;
SEQ ID NO:39: Primer containing a target region (starting base position number 379 of the SALPR sequence):
AAAAAAGCCACGAUAGCCACCAUGAAUAAGGCAGCCAAGCUUCCGGUGCUAUCGGUGGUACUUAUUCCGUCGGGUGUUUCGUCCUUUCCACAA-3′, and
the targeted SALPR-coding region is (coding region base position numbers 379-407, SEQ ID NO:8) 5′-GCCACGATAGCCACCATGAATAAGGCAGC-3′;
SEQ ID NO:40: Primer containing a target region (starting base position number 1732 of the SALPR sequence):
AAAAAAGGGCGCUACGACCUGCUGCCCAGCAGCUCCAAGCUUCGAGCUGCUGGGCAGCAGGUCGUAGCGCCCGGUGUUUCGUCCUUUCCACAA-3′, and
the targeted SALPR-coding region is (coding region base position numbers 1732-1760, SEQ ID NO: 23) 5′-GGGCGCTACGACCTGCTGCCCAGCAGCTC-3′; and
SEQ ID NO:41: Primer containing a target region (starting base position number 1736 of the SALPR sequence):
AAAAAAGCUACGACCUGCUGCCCAGCAGCUCUGCCCAAGCUUCGGCAGAGCUGCUGGGCAGCAGGUCGUAGCGGUGUUUCGUCCUUUCCACAA-3′, and
the targeted SALPR-coding region is (coding region base position numbers 1736-1764, SEQ ID NO:26) 5′-GCTACGACCTGCTGCCCAGCAGCTCTGCC-3′.

### Example XI

### Relaxin-3 siRNA

Sense or antisense siRNAs are designed based upon targeted regions of a DNA sequence, as disclosed herein (see, for example, SEQ ID NO:3, GenBank Accession No. NM_{—}080864), and include fragments having up to 30 bps, 29 bps, 25 bps, 22 bps, 21 bps, 20 bps, 15 bps, 10 bps, 5 bps or any integer thereabout or therebetween. For example, 29 bps siRNA include:
Targeted region (base position numbers 4-32, SEQ ID NO:42) 5′-GCCAGGTACATGCTGCTGCTGCTCCTGGC-3′, the corresponding sense siRNA (SEQ ID NO:43) 5′-GCCAGGUACAUGCUGCUGCUGCUCCUGGC-3′, and the antisense siRNA (SEQ ID NO:44) 5′-GCCAGGAGCAGCAGCAGCAUGUACCUGGC-3′;
Targeted region (base position numbers 77-105, SEQ ID NO:45) 5′-GGGCAGCGCCTTACGGGGTCAGGCTTTGC-3′, the corresponding sense siRNA (SEQ ID NO:46) 5′-GGGCAGCGCCUUACGGGGUCAGGCUUUGC-3′, and the antisense siRNA (SEQ ID NO:47) 5′-GCAAAGCCUGACCCCGUAAGGCGCUGCCC-3′;
Targeted region (base position numbers 122-150, SEQ ID NO:48) 5′-GAGCAGTCATCTTCACCTGCGGGGGCTCC-3′, the corresponding sense siRNA (SEQ ID NO:49) 5′-GAGCAGUCAUCUUCACCUGCGGGGGCUCC-3′, and the antisense siRNA (SEQ ID NO:50) 5′-GGAGCCCCCGCAGGUGAAGAUGACUGCUC-3′; and continuing in this progression to the end of Relaxin-3 coding-sequence, for example,
Targeted region (base position numbers 398-426, SEQ ID NO:51) 5′-GTAGCAAAAGTGAAATCAGTAGCCTTTGC-3′, the corresponding sense siRNA (SEQ ID NO:52) 5′-GUAGCAAAAGUGAAAUCAGUAGCCUUUGC-3′, and the antisense siRNA (SEQ ID NO:53) 5′-CGAAAGGCUACUGAUUUCACUUUUGCUAC-3; and so on as set forth herein.

A set of siRNAs/shRNAs are designed based on Relaxin-3 coding-sequence (see, for example, SEQ ID NO:3, GenBank Accession No. NM_{—}080864).

### Example XII

### A PCR-based Strategy for Cloning Relaxin-3 siRNA/shRNA Sequences

Relaxin-3 oligos can be designed based on a set criteria, for example, 29 bps ‘sense’ sequences (for example, a target region starting base position number 4 of the Relaxin-3 sequence: 5′-GCCAGGTACATGCTGCTGCTGCTCCTGGC-3′, SEQ ID NO:42) containing a ‘C’ at the 3′ end are selected from the Relaxin-3 sequence. A termination sequence (for example, AAAAAA, SEQ ID NO:29), the corresponding antisense Relaxin-3 sequence (for example, 5′-GCCAGGAGCAGCAGCAGCATGTACCTGGC-3′, SEQ ID NO:54), a loop (for example, CAAGCTTC, SEQ ID NO:31), and a reverse primer (for example, U6 reverse primer, GGTGTTTCGTCCTTTCCACAA, SEQ ID NO:32) are subsequently added to the 29 bps sense strands to construct PCR primers (see for example, Paddison et al., Genes & Dev. 16:948-958, 2002). Of course, other sense and anti-sense sequences can be selected from a target molecule to develop siRNAs for that molecule.

Several steps are followed in generating hairpin primers. First, a 29 nt “sense” sequence containing a “C” at the 3′ end is selected. Second, the actual hairpin is constructed in a 5′-3′ orientation with respect to the intended transcript. Third, a few stem pairings are changed to G-U by altering the sense strand sequence. G-U base pairing seems to be beneficial for stability of short hairpins in bacteria and does not interfere with silencing. Finally, the hairpin construct is converted to its “reverse complement” and combined with 21 nt human U6 promoter. See below, an example of the model structures drawn:

A model shRNA structure based on SEQ ID NO:42 is (SEQ ID NO:55):

```
                          5′->3′ Anti-sense strand
-------| GAA
              GCCAGGAGCAGCAGCAGCAUGUACCUGGC G
              CGGUCCUCGUCGUCGUCGUACAUGGACCG C
          UU\hat{ } GUU
                            3′<-5′ Sense strand
```

The linear form of the model (SEQ ID NO:56):

Anti-sense Loop Sense Termination
GCCAGGAGCAGCAGCAGCAUGUACCUGGCGAAGCUUGGCCAGGUACAUGCUGCUGCUGCUCCUGGCUUUUUU

Some base pairing are changed to G-U by altering sense sequence. The final hairpin is converted to its reverse complement.

Hairpin portion of the primer (about 72 nt) (SEQ ID NO:57):

AAAAAACGGUCCUCGUCGUCGUCGUACAUGGACCGCAAGCUUCGCCAGGAGCAGCAGCAGCAUGUACCUGGC
+

U6 promoter (reverse primer sequence): GGUGUUUCGUCCUUUCCACAA (SEQ ID NO:36) Thus, the final hairpin sequence (SEQ ID NO:58) is:

AAAAAACGGUCCUCGUCGUCGUCGUACAUGGACCGCAAGCUUCGCCAGGA
GCAGCAGCAGCAUGUACCUGGCGGUGUUUCGUCCUUUCCACAA

Model shRNA structures also can be developed based on a different set of criteria (see for example, Brummelkamp et al., Science, 296(5567):550-5533, 2002). Thus, Relaxin-3 oligos, siRNA/shRNA also can be designed based sense or anti-sense sequences and the model structure.

PCR and Cloning: A pGEM1 plasmid (Promega) containing the human U6 locus (G. Hannon, CSHL) is used as the template for the PCR reaction. This vector contains about 500 bp of upstream U6 promoter sequence. Since an SP6 sequence flanks the upstream portion of the U6 promoter, an SP6 oligo is used as the universal primer in U6-hairpin PCR reactions. The PCR product is about 600 bp in length. T-A and directional topoisomerase-mediated cloning kits (Invitrogen, Inc. Catalog No. K2040-10, K2400-20) are used according to the manufacturer's instruction.

To obtain stable siRNAs/shRNAs, some nucleotide bases are modified, therefore, the designed oligo sequences may not match the actual coding sequences.

Examples of oligos designed and the targeted base position numbers of the 29 nt sense sequence of the Relaxin-3-coding region (see, for example, SEQ ID NO:3, GenBank Accession No. NM_{—}080864) are shown below:
SEQ ID NO:59: Primer containing a target region (starting base position number 4 of the Relaxin-3 sequence):
AAAAAAGCCAGGUACAUGCUGCUGCUGCUCCUGGCCAAGCUUCGCCAGGAGCAGCAGCAGCAUGUACCUGGCGGUGUUUCGUCCUUUCCACAA-3′, and
the targeted Relaxin-3-coding region is (coding region base position numbers 4-32, SEQ ID NO:42) 5′-GCCAGGTACATGCTGCTGCTGCTCCTGGC-3′;
SEQ ID NO:60: Primer containing a target region (starting base position number 77 of the Relaxin-3 sequence):
AAAAAAGGGCAGCGCCUUACGGGGUCAGGCUUUGCCAAGCUUCGCAAAGCCUGACCCCGUAAGGCGCUGCCCGGUGUUUCGUCCUUUCCACAA-3′, and
the targeted Relaxin-3-coding region is (coding region base position numbers 77-105, SEQ ID NO:45) 5′-GGGCAGCGCCTTACGGGGTCAGGCTTTGC-3′;
SEQ ID NO:61: Primer containing a target region (starting base position number 122 of the Relaxin-3 sequence):
AAAAAGAGCAGUCAUCUUCACCUGCGGGGGCUCCCAAGCUUCGGAGCCCCCGCAGGUGAAGAUGACUGCUCGGUGUUUCGUCCUUUCCACAA-3′, and
the targeted Relaxin-3-coding region is (coding region base position numbers 122-150, SEQ ID NO: 48) 5′-GAGCAGTCATCTTCACCTGCGGGGGCTCC-3′; and
SEQ ID NO:62: Primer containing a target region (starting base position number 398 of the Relaxin-3 sequence):
AAAAAAGUAGCAAAAGUGAAAUCAGUAGCCUUUGCCAAGCUUCCGAAAGGCUACUGAUUUCACUUUUGCUACGGUGUUUCGUCCUUUCCACAA-3′, and
the targeted Relaxin-3-coding region is (coding region base position numbers 398-426, SEQ ID NO:51) 5′-GTAGCAAAAGTGAAATCAGTAGCCTTTGC-3′.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

SEQ ID NO:1. Homo sapiens G-protein coupled receptor (GPCR135); somatostatin- and angiotensin-like peptide receptor (SALPR) sequence (1857 bp). The GenBank Accession No. for Homo sapiens SALPR is NM_{—}016568 (coding region base positions: 361-1770).

| | |
|---|---|
| 1 | GATTTGGGGA GTTATGCGCC AGTGCCCCAG TGACCGCGGG ACACGGAGAG GGGAAGTCTG |
| 61 | CGTTGTACAT AAGGACCTAG GGACTCCGAG CTTGGCCTGA GAACCCTTGG ACGCCGAGTG |
| 121 | CTTGCCTTAC GGGCTGCACT CCTCAACTCT GCTCCAAAGC AGCCGCTGAG CTCAACTCCT |
| 181 | GCGTCCAGGG CGTTCGCTGC GCGCCAGGAC GCGCTTAGTA CCCAGTTCCT GGGCTCTCTC |
| 241 | TTCAGTAGCT GCTTTGAAAG CTCCCACGCA CGTCCCGCAG GCTAGCCTGG CAACAAAACT |
| 301 | GGGGTAAACC GTGTTATCTT AGGTCTTGTC CCCCAGAACA TGACCTAGAG GTACCTGCGC |
| 361 | ATGCAGATGG CCGATGCAGC CACGATAGCC ACCATGAATA AGGCAGCAGG CGGGGACAAG |
| 421 | CTAGCAGAAC TCTTCAGTCT GGTCCCGGAC CTTCTGGAGG CGGCCAACAC GAGTGGTAAC |
| 481 | GCGTCGCTGC AGCTTCCGGA CTTGTGGTGG GAGCTGGGGC TGGAGTTGCC GGACGGCGCG |
| 541 | CCGCCAGGAC ATCCCCCGGG CAGCGGCGGG GCAGAGAGCG CGGACACAGA GGCCCGGGTG |
| 601 | CGGATTCTCA TCAGCGTGGT GTACTGGGTG GTGTGCGCCC TGGGGTTGGC GGGCAACCTG |
| 661 | CTGGTTCTCT ACCTGATGAA GAGCATGCAG GGCTGGCGCA AGTCCTCTAT CAACCTCTTC |
| 721 | GTCACCAACC TGGCGCTGAC GGACTTTCAG TTTGTGCTCA CCCTGCCCTT CTGGGCGGTG |
| 781 | GAGAACGCTC TTGACTTCAA ATGGCCCTTC GGCAAGGCCA TGTGTAAGAT CGTGTCCATG |
| 841 | GTGACGTCCA TGAACATGTA CGCCAGCGTG TTCTTCCTCA CTGCCATGAG TGTGACGCGC |
| 901 | TACCATTCGG TGGCCTCGGC TCTGAAGAGC CACCGGACCC GAGGACACGG CCGGGGCGAC |
| 961 | TGCTGCGGCC GGAGCCTGGG GGACAGCTGC TGCTTCTCGG CCAAGGCGCT GTGTGTGTGG |
| 1021 | ATCTGGGCTT TGGCCGCGCT GGCCTCGCTG CCCAGTGCCA TTTTCTCCAC CACGGTCAAG |
| 1081 | GTGATGGGCG AGGAGCTGTG CCTGGTGCGT TTCCCGGACA AGTTGCTGGG CCGCGACAGG |
| 1141 | CAGTTCTGGC TGGGCCTCTA CCACTCGCAG AAGGTGCTGT TGGGCTTCGT GCTGCCGCTG |
| 1201 | GGCATCATTA TCTTGTGCTA CCTGCTGCTG GTGCGCTTCA TCGCCGACCG CCGCGCGGCG |
| 1261 | GGGACCAAAG GAGGGGCCGC GGTAGCCGGA GGACGCCCGA CCGGAGCCAG CGCCCGGAGA |
| 1321 | CTGTCGAAGG TCACCAAATC AGTGACCATC GTTGTCCTGT CCTTCTTCCT GTGTTGGCTG |
| 1381 | CCCAACCAGG CGCTCACCAC CTGGAGCATC CTCATCAAGT TCAACGCGGT GCCCTTCAGC |
| 1441 | CAGGAGTATT TCCTGTGCCA GGTATACGCG TTCCCTGTGA GCGTGTGCCT AGCGCACTCC |
| 1501 | AACAGCTGCC TCAACCCCGT CCTCTACTGC CTCGTGCGCC GCGAGTTCCG CAAGGCGCTC |
| 1561 | AAGAGCCTGC TGTGGCGCAT CGCGTCTCCT TCGATCACCA GCATGCGCCC CTTCACCGCC |
| 1621 | ACTACCAAGC CGGAGCACGA GGATCAGGGG CTGCAGGCCC CGGCGCCGCC CCACGCGGCC |
| 1681 | GCGGAGCCGG ACCTGCTCTA CTACCCACCT GGCGTCGTGG TCTACAGCGG GGGGCGCTAC |
| 1741 | GACCTGCTGC CCAGCAGCTC TGCCTACTGA CGCAGGCCTC AGGCCCAGGG CGCGCCGTCG |
| 1801 | GGGCAAGGTG GCCTTCCCCG GGCGGTAAAG AGGTGAAAGG ATGAAGGAGG GCTGGGG |

SEQ ID NO:2. Human Somatostatin- and Angiotensin-Like Peptide Receptor (SALPR) Polypeptide Sequence (469 amino acids). The GenBank Protein ID. number is NP_{—}057652.1.

| | |
|---|---|
| 1 | MQMADAATIA TMNKAAGGDK LAELFSLVPD LLEAANTSGN ASLQLPDLWW ELGLELPDGA |
| 61 | PPGHPPGSGG AESADTEARV RILISVVYWV VCALGLAGNL LVLYLMKSMQ GWRKSSINLF |
| 121 | VTNLALTDFQ FVLTLPFWAV ENALDFKWPF GKAMCKIVSM VTSMNMYASV FFLTANSVTR |
| 181 | YHSVASALKS HRTRGHGRGD CCGRSLGDSC CFSAKALCVW IWALAALASL PSAIFSTTVK |
| 241 | VMGEELCLVR FPDKLLGRDR QFWLGLYHSQ KVLLGFVLPL GIIILCYLLL VRFIADRRAA |
| 301 | GTKGGAAVAG GRPTGASARR LSKVTKSVTI VVLSFFLCWL PNQALTTWSI LIKFNAVPFS |
| 361 | QEYFLCQVYA FPVSVCLAHS NSCLNPVLYC LVRREFRKAL KSLLWRIASP SITSMRPFTA |
| 421 | TTKPEHEDQG LQAPAPPHAA AEPDLLYYPP GVVVYSGGRY DLLPSSSAY |

SEQ ID NO:3. Homo sapiens Relaxin-3 (H3) coding sequence (429 bps). The GenBank Accession No. for Homo sapiens Relaxin-3 (H3) is NM_{—}080864.

| | |
|---|---|
| 1 | ATGGCCAGGT ACATGCTGCT GCTGCTCCTG GCGGTATGGG TGCTGACCGG GGAGCTGTGG |
| 61 | CCGGGAGCTG AGGCCCGGGC AGCGCCTTAC GGGGTCAGGC TTTGCGGCCG AGAATTCATC |
| 121 | CGAGCAGTCA TCTTCACCTG CGGGGGCTCC CGGTGGAGAC GATCAGACAT CCTGGCCCAC |
| 181 | GAGGCTATGG GAGATACCTT CCCGGATGCA GATGCTGATG AAGACAGTCT GGCAGGCGAG |
| 241 | CTGGATGAGG CCATGGGGTC CAGCGAGTGG CTGGCCCTGA CCAAGTCACC CCAGGCCTTT |
| 301 | TACAGGGGGC GACCCAGCTG GCAAGGAACC CCTGGGGTTC TTCGGGGCAG CCGAGATGTC |
| 361 | CTGGCTGGCC TTTCCAGCAG CTGCTGCAAG TGGGGGTGTA GCAAAAGTGA AATCAGTAGC |
| 421 | CTTTGCTAG |

SEQ ID NO:4. Human Relaxin-3 preproprotein; insulin-like 7 (INSL7) Polypeptide Sequence (142 amino acids). The GenBank Protein ID. number is NP_{—}543140.1.

| | |
|---|---|
| 1 | MARYMLLLLL AVWVLTGELW PGAEARAAPY GVRLCGREFI PAVIFTCGGS RWRRSDILAH |
| 61 | EANGDTFPDA DADEDSLAGE LDEAMGSSEW LALTKSPQAF YRGRPSWQGT PGVLRGSRDV |
| 121 | LAGLSSSCCK WGCSKSEISS LC |

1. A method for diagnosing a cancer in a mammal, comprising:
a) determining SALPR or Relaxin-3 gene copy number in a test sample from a region of the mammal that is suspected to be precancerous or cancerous, thereby generating data for a test gene copy number; and
b) comparing the test gene copy number to data for a control gene copy number, wherein an amplification of the gene in the test sample relative to the control, respectively, indicates the presence of a precancerous lesion or a cancer in the mammal.

2. The method according to claim 1, wherein the cancer is a lung cancer, a colon cancer, an ovarian cancer, or a pancreatic cancer.

3. A method for inhibiting cancer or precancerous growth in a mammalian tissue, comprising contacting the tissue with an inhibitor that interacts with SALPR or Relaxin-3 protein. SALPR or Relaxin-3 DNA or RNA and thereby inhibits SALPR or Relaxin-3 function, respectively.

4. The method according to claim 3, wherein the tissue is a lung tissue, a colon tissue, an ovarian tissue, or a pancreatic tissue.

5. The method according to claim 3, wherein the inhibitor is a siRNA, miRNA, an antisense RNA, an antisense DNA, a decoy molecule, or a decoy DNA.

6. The method according to claim 3, wherein the inhibitor contains nucleotides, and wherein the inhibitor comprises less than about 100 bps in length.

7. The method according to claim 3, wherein the inhibitor is a ribozyme.

8. The method according to claim 3, wherein the inhibitor is a small molecule.

9-10. (Canceled).

11. A method for diagnosing a cancer in a mammal, comprising:
a) determining the level of SALPR or Relaxin-3 in a test sample from a region of the mammal that is suspected to be precancerous or cancerous, thereby generating data for a test level; and
b) comparing the test level of SALPR or Relaxin-3 to data for a control level, wherein an elevated test level of SALPR or Relaxin-3 of the test sample relative to the control level, respectively, indicates the presence of a precancerous lesion or a cancer in the mammal.

12. The method according to claim 11, wherein the control level is obtained from a database of SALPR or Relaxin-3 levels detected in a control sample.

13. A method of blocking in vivo expression of a gene by administering a vector encoding SALPR or Relaxin-3 siRNA.

14. The method of claim 13, wherein the siRNA interferes with SALPR or Relaxin-3 activity.

15. The method of claim 13, wherein the siRNA causes post-transcriptional silencing of SALPR or Relaxin-3 gene in a mammalian cell.

16. The method of claim 15, wherein the cell is a human cell.

17. A method of screening a test molecule for SALPR or Relaxin-3 antagonist activity comprising the steps of:
a) contacting the molecule with a cancer cell;
b) determining the level of SALPR or Relaxin-3 in the cell, thereby generating data for a test level; and
c) comparing the test level to the SALPR or Relaxin-3 level of the cancer cell prior to contacting the test molecule, respectively, wherein a decrease in SALPR or Relaxin-3 in the test level indicates SALPR or Relaxin-3 antagonist activity of the test molecule.

18. The method of claim 17, wherein the level of SALPR or Relaxin-3 is determined by reverse transcription and polymerase chain reaction (RT-PCR).

19. The method of claim 17, wherein the level of SALPR or Relaxin-3 is determined by Northern hybridization or microarray analysis.

20. The method of claim 17, wherein the cell is obtained from a lung tissue, a colon tissue, an ovarian tissue, or a pancreatic tissue.

21-22. (Canceled).

23. A method of determining whether a test molecule has SALPR or Relaxin-3 antagonist activity, wherein the method comprises:
a) determining the level of SALPR or Relaxin-3 in a test sample containing cancer cells, thereby generating data for a control level;
b) contacting the molecule with the test sample to generate data for a test level; and
c) comparing the control level to the test level, respectively, wherein no decrease in SALPR or Relaxin-3 in the test level as compared to the control level indicates that the test molecule has no SALPR or Relaxin-3 antagonist activity.

24. (Canceled).

25. A method for determining the efficacy of a therapeutic treatment regimen in a patient, comprising:
a) measuring at least one of SALPR or Relaxin-3 gene copy number, SALPR or Relaxin-3 mRNA, or SALPR or Relaxin-3 expression levels in a first sample obtained from a patient, thereby generating an initial level;
b) administering the treatment regimen to the patient;
c) measuring at least one of SALPR or Relaxin-3 gene copy number, SALPR or Relaxin-3 mRNA, or SALPR or Relaxin-3 expression levels in a second sample from the patient at a time following administration of the treatment regimen, thereby generating a test level; and
d) comparing the initial and test levels, respectively, wherein a decrease in the gene copy number, SALPR or Relaxin-3 mRNA, or SALPR or Relaxin-3 expression level in the test level relative to the initial level indicates that the treatment regimen is effective in the patient.

26. The method according to claim 25, wherein the sample is obtained from a lung tissue, a colon tissue, an ovarian tissue, or a pancreatic tissue.

27-31. (Canceled).
